(19) United States
(12) Patent Application Publication
(10) Pub. No.: US 2011/0263610 A1
(43) Pub. Date: Oct. 27, 2011

(54) COMPOUNDS AND COMPOSITIONS AS ITPKB INHIBITORS

(75) Inventors: Yongqin Wan, Irvine, CA (US); Shifeng Pan, San Diego, CA (US); Guobao Zhang, San Diego, CA (US); Xia Wang, San Diego, CA (US); Yun Feng Xie, San Diego, CA (US); Jiqing Jiang, San Diego, CA (US); Dean Phillips, San Marcos, CA (US); Yang Yang, San Diego, CA (US)

(73) Assignee: IRM LLC, Hamilton (BM)

(21) Appl. No.: 12/935,273

(22) PCT Filed: Mar. 30, 2009
(86) PCT No.: PCT/US09/38734

§ 371 Date: Dec. 16, 2010

Related U.S. Application Data
(60) Provisional application No. 61/042,369, filed on Apr. 4, 2008.

(51) Int. Cl. C07D 487/04; C07D 405/14; A61P 19/02; A61P 17/06; A61P 37/00; A61K 31/496; C07D 401/14

(52) U.S. Cl. 514/253.05; 514/253.04; 514/253.09; 544/362; 544/364; 544/363

(57) ABSTRACT

The invention provides a novel class of compounds, pharmaceutical compositions comprising such compounds and methods of using such compounds to treat or prevent diseases or disorders associated with abnormal or deregulated B cell activities, particularly diseases or disorders that involve aberrant activation of inositol 1,4,5-trisphosphate 3-kinase B (ITPKb).

### CROSS-REFERENCE TO RELATED APPLICATIONS

This application claims the benefit of priority under 35 U.S.C. §119(e) to U.S. Provisional Patent Application No. 61/042,369, filed Apr. 4, 2008, the disclosure of which is incorporated herein by reference in its entirety and for all purposes.

### FIELD OF THE INVENTION

The invention relates to compounds, pharmaceutical compositions comprising such compounds and methods of using such compounds to treat or prevent diseases or disorders associated with abnormal or deregulated B cell activities, particularly diseases or disorders that involve aberrant activation of inositol 1,4,5-trisphosphate 3-kinase B (ITPKb).

### BACKGROUND OF THE INVENTION

Inositol 1,4,5-trisphosphate 3-kinase B (ITPKB) is one of three inositol trisphosphate kinases (ITPKA, ITPKB and ITPKC) that convert inositol 1,4,5-trisphosphate (IP₃) to inositol 1,3,4,5-tetrakisphosphate (IP₄). Inositol 1,4,5-trisphosphate 3-kinase B (ITPKB) is a protein encoded by the human gene itpkb and the activity of this encoded protein is responsible for regulating the levels of a large number of inositol polyphosphates that are important in cellular signaling. Unlike protein kinases, ITPKB does not phosphorylate other proteins, rather ITPKB regulates inositol phosphate metabolism by phosphorylation of second messenger inositol 1,4,5-trisphosphate (IP₃) to inositol 1,3,4,5-tetrakisphosphate (IP₄). ITPKB alone is uniquely required for lymphocyte development and activation. ITPKB activity is controlled by both calcium/calmodulin and protein phosphorylation mechanisms.

### SUMMARY OF THE INVENTION

Provided herein are compounds and pharmaceutical compositions thereof, which are useful modulators of the activity of ITPKb and are useful in the treatment and/or prevention of ITPKb-associated diseases.

In one aspect, the compounds, and the pharmaceutically acceptable salts, pharmaceutically acceptable solvates (e.g. hydrates), the N-oxide derivatives, prodrug derivatives, protected derivatives, individual isomers and mixture of isomers thereof, provided herein, have a structure according to Formula (I):

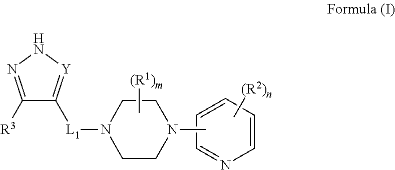

wherein:

L₁ is —(CR¹¹R¹²)ₚ—, —C(O)—, or —S(O)₂—;

L₂ is —C(O)—, —C(O)NR⁵— or —NR⁵C(O;

Y is N or CR⁴;

each R¹ is independently selected from —C(O)R⁹, C₁-C₆alkyl, C₁-C₆heteroalkyl, C₁-C₆haloalkyl, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, aryl, heteroaryl, C₃-C₈cycloalkyl, and C₃-C₁₀heterocycloalkyl groups of R¹ are each optionally substituted with 1 to 3 substituents independently selected from halogen, —CN, C₁-C₆alkyl, C₁-C₆haloalkyl, C₁-C₆alkoxy, C₁-C₆haloalkoxyl, C₃-C₈cycloalkyl, C₃-C₁₀heterocycloalkyl, —OR⁹, —C(O)R⁹, —OC(O)R⁹, —C(O)OR⁹, —N(R⁶R⁷⁾, —C(O)N(R⁶R⁷), —S(O)₂R⁹, —S(O)₂N(R⁶R⁷), and —NR⁷S(O)₂R⁹;
or two R₁ groups are each independently C₁-C₄alkyl and form a C₁-C₄alkyl bridge, or two R₁ groups are each independently C₁-C₄alkyl and taken together with the C atom to which they are attached form an optionally substituted C₃-C₈cycloalkyl;
each R² is independently selected from halogen, —CN, —OR⁹, —C(O)R⁹, —C(O)N(R⁶R⁷), C₁-C₆alkyl, C₁-C₆heteroalkyl, C₁-C₆haloalkyl, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, aryl, heteroaryl, C₃-C₈cycloalkyl, and C₃-C₁₀heterocycloalkyl groups of R² are each optionally substituted with 1 to 3 substituents independently selected from halogen, —CN, C₁-C₆alkyl, C₁-C₆haloalkyl, C₁-C₆ alkoxy, C₁-C₆haloalkoxyl, C₃-C₈cycloalkyl, C₃-C₁₀heterocycloalkyl, —OR⁹, —C(O)R⁹, —OC(O)9-C(O)OR⁹, —N(R⁶R⁷), —C(O)N(R⁶R⁷), —S(O)₂R⁹, —S(O)₂N(R⁶R⁷), and —NR⁷S(O)₂R⁹;
when Y is N then R³ is selected from L₂-R¹⁰, C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, C₃-C₈cycloalkyl, C₃-C₁₀heterocycloalkyl, C₆₋₁₀aryl and C₂-C₉heteroaryl, wherein the C₁-C₆alkyl, C₁-C₆heteroalkyl, C₁-C₆haloalkyl, C₁-C₆alkoxy, C₁-C₆haloalkoxy, C₂-C₉heteroaryl, C₃-C₈cycloalkyl, aryl and C₃-C₁₀heterocycloalkyl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —NR⁶C(O)R⁷, —C(O)N(R⁶R⁷), —S(O)₂R⁹, —S(O)₂N(R⁶R⁷) and —NR⁷S(O)₂R⁹;
when Y is CR⁴ then R³ is selected from L₂-R¹⁰, C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, C₃-C₈cycloalkyl, C₃-C₁₀heterocycloalkyl and C₂-C₉heteroaryl, provided that R³ is not a six-membered heteroaryl containing 1 to 3 N atoms, and wherein the C₁-C₆alkyl, C₁-C₆heteroalkyl, C₁-C₆haloalkyl, C₁-C₆alkoxy, C₁-C₆haloalkoxy, C₂-C₉heteroaryl, C₃-C₈cycloalkyl and C₃-C₁₀heterocycloalkyl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —NR⁶C(O)R⁷, —C(O)N(R⁶R⁷), —S(O)₂R⁹, —S(O)₂N(R⁶R⁷) and —NR⁷S(O)₂R⁹;
R⁴ is selected from H, —C(O)OR⁹, —C(O)R⁹, —C(O)N(R⁶R⁷), —N(R⁶R⁷), —NR⁶C(O)R⁷, —(CH₂)ₙOR⁷, C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₁₀heterocycloalkyl groups of R⁵ are each optionally substituted with 1 to 3 substituents independently selected from halogen, —CN, —R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷), and —NR⁷S(O)₂R⁹;
R⁵, R⁶ and R⁷ are each independently selected from H, C₁-C₆alkyl, C₁-C₆haloalkyl, C₁-C₆alkoxy, C₃-C₈cycloalkyl, C₃-C₁₀heterocycloalkyl, C₁-C₆haloalkyl, C₁-C₆haloalkoxy, aryl and heteroaryl, wherein the C₁-C₆alkyl, C₁-C₆halealkyl, C₁-C₆alkoxy, C₃-C₈cycloalkyl, C₃-C₁₀heterocycloalkyl, C₁-C₆haloalkyl, C₁-C₆haloalkoxy, aryl and heteroaryl of R⁵, R⁶ and R⁷ are each optionally substituted with 1 to 3 substituents independently selected from halogen, —CN, —R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷), and —NR⁷S(O)₂R⁹,
or R⁶ and R⁷ are each independently C₁-C₄alkyl and taken together with the C atom to which they are attached form a C₃-C₈cycloalkyl;
R⁸ is selected from H, CN, —OR⁹, —C(O)R⁹, —C(O)OR⁹, —C(O)N(R⁶R⁷), —C(═NH)N(R⁶R⁷), C₁-C₆alkyl, C₁-C₆haloalkyl, C₁-C₆alkoxy, C₁-C₆haloalkoxy, C₃-C₈cycloalkyl, and C₃-C₁₀heterocycloalkyl;
R⁹ is selected from H, C₁-C₆alkyl, C₁-C₆haloalkyl, C₁-C₆alkoxy, C₃-C₈cycloalkyl, C₃-C₁₀heterocycloalkyl, C₁-C₆haloalkyl and C₁-C₆haloalkoxy;
R¹⁰ is selected from C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, C₁-C₆alkoxy, C₁-C₆haloalkoxy, aryl, heteroaryl, C₃-C₈cycloalkyl, and C₃-C₁₀heterocycloalkyl groups of R¹¹ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁷, —S(O)₂N(R⁶R⁷) and —NR⁷S(O)₂R⁹;
R¹¹ and R¹² are each independently selected from H, C₁-C₄alkyl, C₁-C₄heteroalkyl, C₁-C₄haloalkyl, C₁-C₄alkoxy and C₁-C₄haloalkoxy;
or R¹¹ and R¹² are each independently C₁-C₄alkyl and taken together with the C atom to which they are attached form a C₃-C₈cycloalkyl;
m is, independently at each occurrence, 0, 1, 2, 3 or 4;
n is, independently at each occurrence, 0, 1, 2, 3 or 4, and
p is, independently at each occurrence, 1, 2, 3 or 4.

In certain embodiments, such compounds of Formula (I), have a structure according to Formula (II):

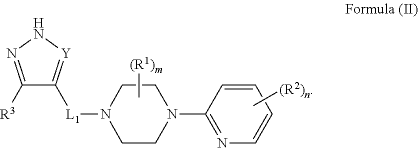

In certain embodiments, n is 0, 1 or 2, while in other embodiments, such compounds of Formulas (I)-(II), have a structure according to Formula (III):

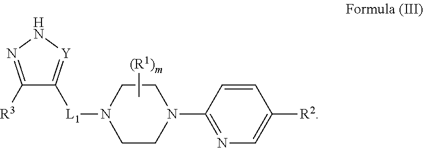

In certain embodiments, m is 0, 1 or 2, while in other embodiments, such compounds of Formulas (I)-(III), have a structure according to Formula (IV) or Formula (V):

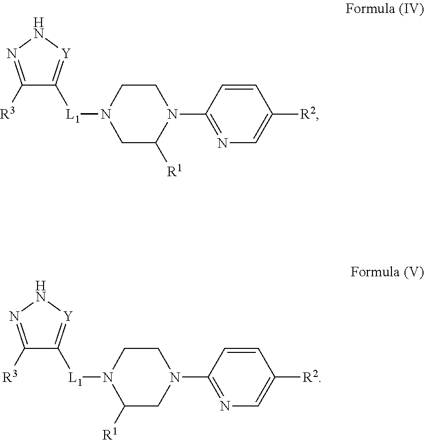

In certain embodiments of such compounds of Formulas (I)-(V), L₁ is —(CR¹¹R¹²)ₚ—. In other embodiments of such compounds of Formulas (I)-(V), R¹¹ and R¹² are each independently selected from H and C₁-C₄alkyl.

In other embodiments of such compounds of Formulas (I)-(V), L₁ is —(CH₂)— and such compounds have a structure according to Formula (VI) or Formula (VII):

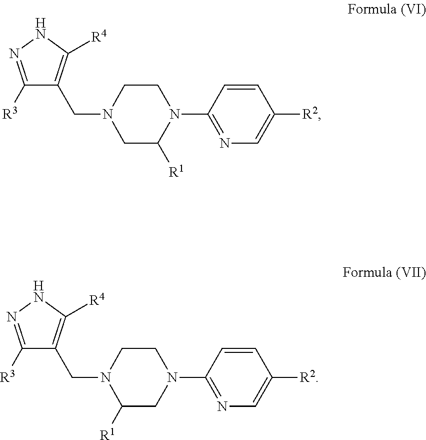

In other embodiments of such compounds of Formulas (I)-(VII), R⁴ is H, and such compounds have a structure according to Formula (VIII) or Formula (IX):

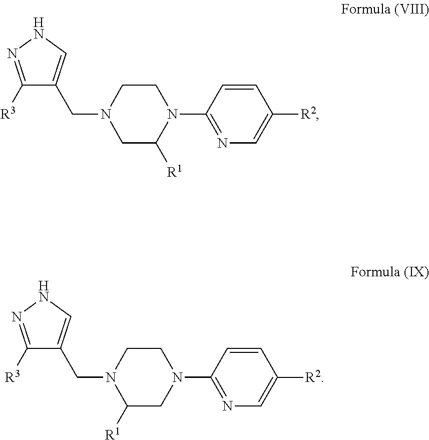

In other embodiments of such compounds of Formulas (I)-(V), L₁ is —(CH₂)— and such compounds have a structure according to Formula (X) or Formula (XI):

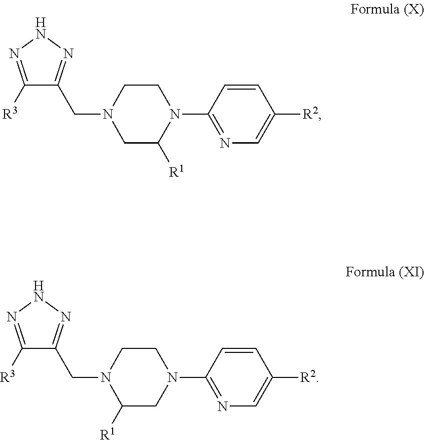

In other embodiments of such compounds of Formulas (I)-(XI), R¹ is C₁-C₆alkyl or C₁-C₆haloalkyl, while in other embodiments of such compounds of Formulas (I)-(XI), R² is C₁-C₆alkyl or C₁-C₆haloalkyl. In certain embodiments of such compounds of Formulas (I)-(XI), R¹ is methyl, ethyl, trifluoromethyl, difluoromethyl or fluoromethyl, while in other embodiments of such compounds of Formulas (I)-(XI), R² is methyl, ethyl, trifluoromethyl, difluoromethyl or fluoromethyl.

In other embodiments, such compounds of Formulas (I) have a structure according to Formula (XII), Formula (XIII), Formula (XIV) or Formula (XV):

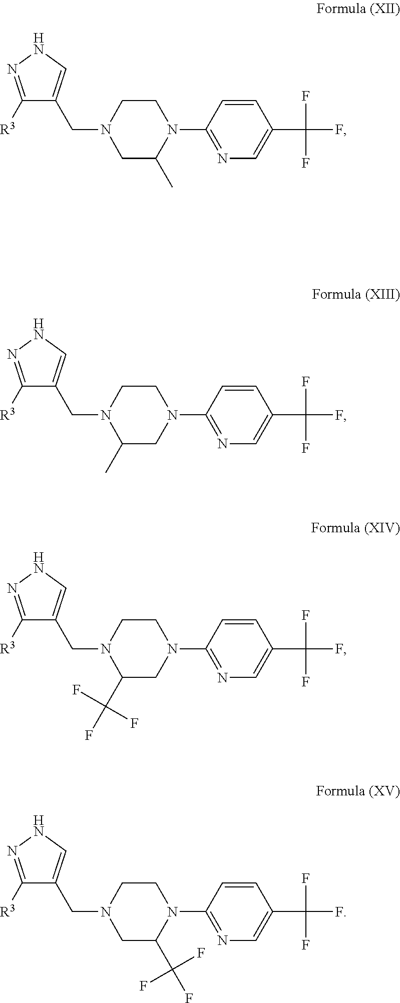

In other embodiments, such compounds of Formulas (I) have a structure according to Formula (XVI), Formula (XVII), Formula (XVIII) or Formula (XIX):

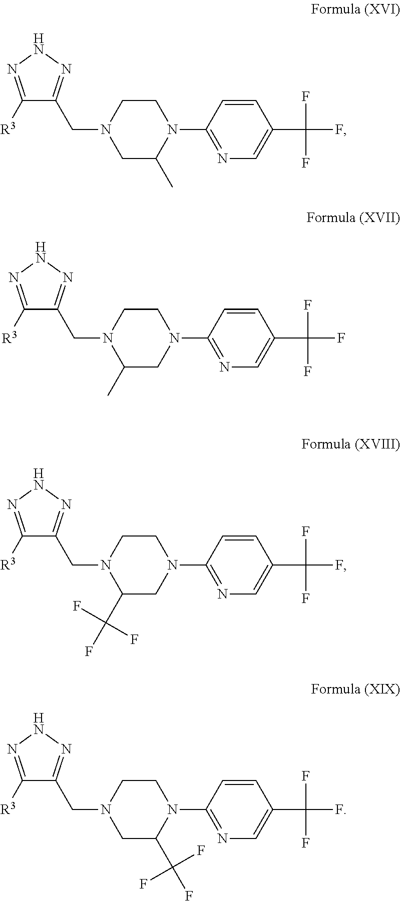

In certain embodiments of such compounds of Formulas (I)-(XIX) when Y is CR⁴, then R³ is C₃-C₁₀heterocycloalkyl or C₂-C₉heteroaryl, wherein the C₃-C₁₀heterocycloalkyl and C₂-C₉heteroaryl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷) and —NR⁷S(O)₂R⁹ and provided that R³ is not a six-membered heteroaryl containing 1 to 3 N atoms. In certain embodiments of such compounds of Formulas (I)-(XIX) when Y is CR⁴, then the C₃-C₁₀heterocycloalkyl and C₂-C₉heteroaryl groups of R³ are substituted with R⁸. In certain embodiments of such compounds of Formulas (I)-(XIX) when Y is CR⁴, the C₂-C₉heteroaryl is selected from benzofuranyl, benzofurazanyl, benzoxazolyl, benzopyranyl, benzthiazolyl, benzothienyl, benzazepinyl, benzimidazolyl, benzothiopyranyl, benzo[1,3]dioxole, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, quinoxalinyl, quinolinyl, quinazolinyl, 4H-quinolizinyl, thiazolyl, thiadiazolyl, thienyl, triazolyl and tetrazolyl.

In certain embodiments of such compounds of Formulas (I)-(XIX) when Y is N, then R³ is aryl, C₃-C₁₀heterocycloalkyl or C₂-C₉heteroaryl, wherein the aryl, C₃-C₁₀heterocycloalkyl and C₂-C₉heteroaryl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷) and —NR⁷S(O)₂R⁹. In certain embodiments of such compounds of Formulas (I)-(XIX) when Y is N, then the C₃-C₁₀heterocycloalkyl and C₂-C₉heteroaryl groups of R³ are substituted with R⁸. In certain embodiments of such compounds of Formulas (I)-(XIX) when Y is N, the C₂-C₉heteroaryl is selected from benzofuranyl, benzofurazanyl, benzoxazolyl, benzopyranyl, benzthiazolyl, benzothienyl, benzazepinyl, benzimidazolyl, benzothiopyranyl, benzo[1,3]dioxole, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, pyridazinyl, pyrazinyl, pyrimidinyl, quinoxalinyl, quinolinyl, quinazolinyl, 4H-quinolizinyl, thiazolyl, thiadiazolyl, thienyl, triazinyl, triazolyl and tetrazolyl.

In certain embodiments of such compounds of Formulas (I)-(XIX), R³ is L₂-R¹⁰, while in other embodiments L₂ is selected from C₁-C₆alkenylene, —C(O)— and —C(O)NR⁵, and in other embodiments R¹⁰ is selected from aryl, heteroaryl and C₃-C₁₀heterocycloalkyl, wherein the aryl, heteroaryl and C₃-C₁₀heterocycloalkyl groups of R¹⁰ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁷, —S(O)₂N(R⁶R⁷) and —NR⁷S(O)₂R⁹. In still other embodiments, of such compounds of Formulas (I)-(XIX), R¹⁰ is selected from aryl, heteroaryl and C3-C10heterocycloalkyl, wherein the aryl, heteroaryl and C₃-C₁₀heterocycloalkyl groups of R¹⁰ are substituted with R⁸.

In certain embodiments of such compounds of Formulas (I)-(XIX), R⁸ is selected from CN, —OR⁹, —C(O)R⁹⁰, —C(O)OR⁹, —C(O)N(R⁶R⁷), and —C(═NH)N(R⁶R⁷).

In certain embodiments of such compounds of Formulas (I)-(XIX), R³ is selected from isoquinoline, 2-oxo-1,2-dihydropyridine-4-carbonitrile, thiophene, pyrrole, 1H-pyrrole-3-carbonitrile, phenyl, benzimidazole, 5-phenyl-1H-imidazole, 5-fluoro-1H-benzo[d]imidazole, 4,5,6,7-tetrahydro-1H-benzo[d]imidazole, imidazole, 5-methyl-1H-imidazole, 4,5-dimethyl-1H-imidazol, 1H-imidazo[4,5-c]pyridine, 4-(trifluoromethyl)-1H-imidazole, 1H-benzo[d]imidazole-5-carbonitrile, 1H-imidazole-4-carbonitrile, 1H-pyrrole-3-carboxamide, 1H-pyrrole-2-carboxamide, 1H-pyrrole-2-carbonitrile, furan-2-carboxylic acid, furan-2-carboxamide, furan-3-carboxamide, methyl furan-2-carboxylate, N-methyl-1H-pyrrole-3-carboxamide, 1H-pyrrolo[2,3-b]pyridine, N,N-dimethyl-1H-pyrrole-3-carboxamide, N-(2-hydroxypropyl)-1H-pyrrole-3-carboxamide, (S)—N-(1-hydroxypropan-2-yl)-1H-pyrrole-3-carboxamide, 1H-indole, N-(2-hydroxyethyl)-1H-pyrrole-3-carboxamide, 1,2,3,6-tetrahydropyridine, 5,6-dihydropyridine-1(2H)-carbaldehyde, 1-(5,6-dihydropyridin-1(2H)-yl)ethanone, 1-(5,6-dihydropyridin-1(2H)-yl)-3-hydroxypropan-1-one, piperidine, 1-(piperidin-1-yl)ethanone, piperidine-1-carbaldehyde, 1H-imidazole-4-carboximidamide and 1H-imidazole-4-carboxamide. In other embodiments of such compounds of Formulas (I)-(XIX), R⁵ is H or C₁-C₆alkyl.

In certain embodiments of such compounds of Formulas (I)-(XIX), L₂ is —C(O)NR⁵— and R¹⁰ is selected from 2H-benzo[b][1,4]oxazin-3(4H)-one, 1-phenyl-1H-imidazole, N-(5-methylisoxazol-3-yl)benzenesulfonamide, 1H-indole, 1H-imidazole-5-carbonitrile, 3-(furan-2-yl)-1H-pyrazole, N,N-dimethyl-2-(3-methyl-1H-pyrazol-1-yl)ethanamine and 1H-pyrazole-4-carbonitrile.

In certain embodiments of such compounds of Formulas (I)-(XIX), The compound of any of claims 1-24, wherein L₂ is —C(O)— and R¹⁰ is selected from azetidin-3-ol, pyrrolidin-3-ol and piperidin-4-ol.

In certain embodiments of such compounds of Formulas (I)-(XIX), R⁶ is H or C₁-C₆alkyl, while in other embodiments of such compounds of Formulas (I)-(XIX), R⁷ is H or C₁-C₆alkyl. In certain embodiments of such compounds of Formulas (I)-(XIX), R⁹ is H or C₁-C₆alkyl.

In certain embodiments, the compounds of Formula (I) are (R)-4-((3-methyl-4-(5-(trifluoromethyl)pyridin-2-yl)piperazin-1-yl)methyl)-N-(pyridin-4-yl)-1H-pyrazole-3-carboxamide; (R)-5-(4-((3-methyl-4-(5-(trifluoromethyl)pyridin-2-yl)piperazin-1-yl)methyl)-1H-pyrazol-3-yl)-1H-pyrrole-3-carbonitrile; (R)-5-(4-((3-methyl-4-(5-(trifluoromethyl)pyridin-2-yl)piperazin-1-yl)methyl)-1H-pyrazol-3-yl)-1H-pyrrole-3-carboxamide; (R)—N-methyl-5-(4-((3-methyl-4-(5-(trifluoromethyl)pyridin-2-yl)piperazin-1-yl)methyl)-1H-pyrazol-3-yl)-1H-pyrrole-3-carboxamide; (R)-5-(4-((3-methyl-4-(5-(trifluoromethyl)pyridin-2-yl)piperazin-1-yl)methyl)-1H-pyrazol-3-yl)-1H-pyrrole-2-carbonitrile; (R)-5-(4-((3-methyl-4-(5-(trifluoromethyl)pyridin-2-yl)piperazin-1-yl)methyl)-1H-pyrazol-3-yl)-1H-pyrrole-2-carboxamide; (R)-4-(4-((3-methyl-4-(5-(trifluoromethyl)pyridin-2-yl)piperazin-1-yl)methyl)-1H-pyrazol-3-yl)-1H-pyrrole-2-carbonitrile; (R)-4-(4-((3-methyl-4-(5-(trifluoromethyl)pyridin-2-yl)piperazin-1-yl)methyl)-1H-pyrazol-3-yl)-1H-pyrrole-2-carboxamide; (R)-2-(4-((3-methyl-4-(5-(trifluoromethyl)pyridin-2-yl)piperazin-1-yl)methyl)-1H-pyrazol-3-yl)-1H-indole; (R)-2-methyl-4-((3-(1,2,3,6-tetrahydropyridin-4-yl)-1H-pyrazol-4-yl)methyl)-1-(5-(trifluoromethyl)pyridin-2-yl)piperazine; (R)-4-(4-((3-methyl-4-(5-(trifluoromethyl)pyridin-2-yl)piperazin-1-yl)methyl)-1H-pyrazol-3-yl)-5,6-dihydropyridine-1(2H)-carbaldehyde; (R)-2-methyl-4-((3-(4-(trifluoromethyl)-1H-imidazol-2-yl)-1H-pyrazol-4-yl)methyl)-1-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-1H-imidazole-4-carboximidamide; (R)-2-(4-((3-methyl-4-(5-(trifluoromethyl)pyridin-2-yl)piperazin-1-yl)methyl)-1H-pyrazol-3-yl)-1H-imidazo[4,5-c]pyridine; (R,Z)-5-((4-((3-methyl-4-(5-(trifluoromethyl)pyridin-2-yl)piperazin-1-yl)methyl)-1H-pyrazol-3-yl)methylene)imidazolidine-2,4-dione; (R)-6-(4-((3-methyl-4-(5-(trifluoromethyl)pyridin-2-yl)piperazin-1-yl)methyl)-1H-pyrazol-3-yl)isoquinoline; (R)-6-(4-((3-methyl-4-(5-(trifluoromethyl)pyridin-2-yl)piperazin-1-yl)methyl)-1H-pyrazol-3-yl)-2-oxo-1,2-dihydropyridine-3-carbonitrile; 1-((3-(thiophen-2-yl)-1H-pyrazol-4-yl)methyl)-4-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-1H-benzo[d]imidazole; (R)-2-methyl-4-((3-(5-methyl-1H-imidazol-2-yl)-1H-pyrazol-4-yl)methyl)-1-(5-(trifluoromethyl)pyridin-2-yl)piperazine; (R)-4-((3-(4,5-dimethyl-1H-imidazol-2-yl)-1H-pyrazol-4-yl)methyl)-2-methyl-1-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-4,5,6,7-tetrahydro-1H-benzo[d]imidazole; (R)-5-fluoro-2-(4-((3-methyl-4-(5-(trifluoromethyl)pyridin-2-yl)piperazin-1-yl)methyl)-1H-pyrazol-3-yl)-1H-benzo[d]imidazole; (R)-2-(4-((3-methyl-4-(5-(trifluoromethyl)pyridin-2-yl)piperazin-1-yl)methyl)-1H-pyrazol-3-yl)-1H-benzo[d]imidazole-5-carbonitrile; (S)-5-(4-((3-(trifluoromethyl)-4-(5-(trifluoromethyl)pyridin-2-yl)piperazin-1-yl)methyl)-1H-pyrazol-3-yl)-1H-pyrrole-3-carbonitrile; (R)—N-(5-cyano-1H-imidazol-4-yl)-4-((3-methyl-4-(5-(trifluoromethyl)pyridin-2-yl)piperazin-1-yl)methyl)-1H-pyrazole-3-carboxamide; (R)—N-(3-(furan-2-yl)-1H-pyrazol-5-yl)-4-((3-methyl-4-(5-(trifluoromethyl)pyridin-2-yl)piperazin-1-yl)methyl)-1H-pyrazole-3-carboxamide; (R)—N-(1-(2-(dimethylamino)ethyl)-3-methyl-1H-pyrazol-5-yl)-4-4-((3-methyl-4-(5-(trifluoromethyl)pyridin-2-yl)piperazin-1-yl)methyl)-1H-pyrazole-3-carboxamide; (R)—N-(4-cyano-1H-pyrazol-3-yl)-4-((3-methyl-4-(5-(trifluoromethyl)pyridin-2-yl)piperazin-1-yl)methyl)-1H-pyrazole-3-carboxamide; (R)-(3-hydroxyazetidin-1-yl)(4-((3-methyl-4-(5-(trifluoromethyl)pyridin-2-yl)piperazin-1-yl)methyl)-1H-pyrazol-3-yl)methanone; (3-hydroxypyrrolidin-1-yl)(4-(((R)-3-methyl-4-(5-(trifluoromethyl)pyridin-2-yl)piperazin-1-yl)methyl)-1H-pyrazol-3-yl)methanone; (R)-(4-hydroxypiperidin-1-yl)(4-((3-methyl-4-(5-(trifluoromethyl)pyridin-2-yl)piperazin-1-yl)methyl)-1H-pyrazol-3-yl)methanone; (R)-methyl 5-(4-((3-methyl-4-(5-(trifluoromethyl)pyridin-2-yl)piperazin-1-yl)methyl)-1H-pyrazol-3-yl)furan-2-carboxylate; (R)-5-(4-((3-methyl-4-(5-(trifluoromethyl)pyridin-2-yl)piperazin-1-yl)methyl)-1H-pyrazol-3-yl)furan-2-carboxylic acid; (R)-5-(4-((3-methyl-4-(5-(trifluoromethyl)pyridin-2-yl)piperazin-1-yl)methyl)-1H-pyrazol-3-yl)furan-2-carboxamide; (R)-4-(4-((3-methyl-4-(5-(trifluoromethyl)pyridin-2-yl)piperazin-1-yl)methyl)-1H-pyrazol-3-yl)-1H-pyrrolo[2,3-b]pyridine; (R)—N,N-dimethyl-5-(4-((3-methyl-4-(5-(trifluoromethyl)pyridin-2-yl)piperazin-1-yl)methyl)-1H-pyrazol-3-yl)-1H-pyrrole-3-carboxamide; (R)—N-(2-hydroxyethyl)-5-(4-((3-methyl-4-(5-(trifluoromethyl)pyridin-2-yl)piperazin-1-yl)methyl)-1H-pyrazol-3-yl)-1H-pyrrole-3-carboxamide; N-(2-hydroxypropyl)-5-(4-(((R)-3-methyl-4-(5-(trifluoromethyl)pyridin-2-yl)piperazin-1-yl)methyl)-1H-pyrazol-3-yl)-1H-pyrrole-3-carboxamide; (R)-3-(4-((3-methyl-4-(5-(trifluoromethyl)pyridin-2-yl)piperazin-1-yl)methyl)-1H-pyrazol-3-yl)-1H-pyrrolo[2,3-b]pyridine; N-((S)-1-hydroxypropan-2-yl)-5-(4-(((R)-3-methyl-4-(5-(trifluoromethyl)pyridin-2-yl)piperazin-1-yl)methyl)-1H-pyrazol-3-yl)-1H-pyrrole-3-carboxamide; (R,Z)-5-((4-((3-methyl-4-(5-(trifluoromethyl)pyridin-2-yl)piperazin-1-yl)methyl)-1H-pyrazol-3-yl)methylene)-2-thioxoimidazolidin-4-one; (R)-1-(4-(4-((3-methyl-4-(5-(trifluoromethyl)pyridin-2-yl)piperazin-1-yl)methyl)-1H-pyrazol-3-yl)-5,6-dihydropyridin-1(2H)-yl)ethanone; (R)-2-methyl-4-((3-(piperidin-4-yl)-1H-pyrazol-4-yl)methyl)-1-(5-(trifluoromethyl)pyridin-2-yl)piperazine; (R)-4-(4-((3-methyl-4-(5-(trifluoromethyl)pyridin-2-yl)piperazin-1-yl)methyl)-1H-pyrazol-3-yl)piperidine-1-carbaldehyde; (R,Z)-2-imino-5-((4-((3-methyl-4-(5-(trifluoromethyl)pyridin-2-yl)piperazin-1-yl)methyl)-1H-pyrazol-3-yl)methylene)imidazolidin-4-one; (S)-5-(4-((3-(trifluoromethyl)-4-(5-(trifluoromethyl)pyridin-2-yl)piperazin-1-yl)methyl)-1H-pyrazol-3-yl)-1H-pyrrole-3-carboxamide; (R)-5-(4-((3-methyl-4-(5-(trifluoromethyl)pyridin-2-yl)piperazin-1-yl)methyl)-1H-pyrazol-3-yl)furan-3-carboxylic acid; (R)-5-(4-((3-methyl-4-(5-(trifluoromethyl)pyridin-2-yl)piperazin-1-yl)methyl)-1H-pyrazol-3-yl)furan-3-carboxamide; (R)-3-hydroxy-1-(4-(4-((3-methyl-4-(5-(trifluoromethyl)pyridin-2-yl)piperazin-1-yl)methyl)-1H-pyrazol-3-yl)-5,6-dihydropyridin-1(2H)-yl)propan-1-one; (R)-6-(4-((3-methyl-4-(5-(trifluoromethyl)pyridin-2-yl)piperazin-1-yl)methyl)-1H-pyrazol-3-yl)-2-oxo-1,2-dihydropyridine-4-carbonitrile; (R)-2-methyl-4-((3-(5-phenyl-1H-imidazol-2-yl)-1H-pyrazol-4-yl)methyl)-1-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-1H-imidazole-4-carbonitrile; (R)-4-((3-methyl-4-(5-(trifluoromethyl)pyridin-2-yl)piperazin-1-yl)methyl)-N-(3-oxo-3,4-dihydro-2H-benzo[b][1,4]oxazin-7-yl)-1H-pyrazole-3-carboxamide; (R)-4-((3-methyl-4-(5-(trifluoromethyl)pyridin-2-yl)piperazin-1-yl)methyl)-N-(3-oxo-3,4-dihydro-2H-benzo[b][1,4]oxazin-6-yl)-1H-pyrazole-3-carboxamide; (R)—N-(4-(1H-imidazol-1-yl)phenyl)-4-((3-methyl-4-(5-(trifluoromethyl)pyridin-2-yl)piperazin-1-yl)methyl)-1H-pyrazole-3-carboxamide; (R)-4-((3-methyl-4-(5-(trifluoromethyl)pyridin-2-yl)piperazin-1-yl)methyl)-N-(4-(N-(5-methylisoxazol-3-yl)sulfamoyl)phenyl)-1H-pyrazole-3-carboxamide; (R)-3-(4-((3-methyl-4-(5-(trifluoromethyl)pyridin-2-yl)piperazin-1-yl)methyl)-1H-pyrazol-3-yl)-1H-indole; (R)-2-(4-((3-methyl-4-(5-(trifluoromethyl)pyridin-2-yl)piperazin-1-yl)methyl)-1H-pyrazol-3-yl)-1H-imidazole-4-carboxamide; (R)-4-(5-((3-methyl-4-(5-(trifluoromethyl)pyridin-2-yl)piperazin-1-yl)methyl)-2H-1,2,3-triazol-4-yl)benzonitrile and (R)-4-(5-((3-methyl-4-(4-(trifluoromethyl)phenyl)piperazin-1-yl)methyl)-2H-1,2,3-triazol-4-yl)benzonitrile.

Another aspect provided herein are pharmaceutical compositions include a therapeutically effective amount of a compound of Formulas (I)-(XIX) and a pharmaceutically acceptable carrier. In certain embodiments of such pharmaceutical compositions the pharmaceutical composition is formulated for intravenous administration, intramuscular administration, oral administration, rectal administration inhalation, nasal administration, topical administration, ophthalmic administration or otic administration. In other embodiments of such pharmaceutical compositions the pharmaceutical composition is a tablet, a pill, a capsule, a liquid, an inhalant, a nasal spray solution, a suppository, a solution, an emulsion, an ointment, eye drop or ear drop. In other embodiments of such pharmaceutical compositions further include one or more additional therapeutic agents.

Another aspect provided herein are medicaments for treating or preventing a disease or disorder where modulation of 1,4,5,-trisphosphate 3 kinase B (ITPKB) is implicated, wherein such medicaments include a therapeutically effective amount of a compound of Formulas (I)-(XIX).

Another aspect provided herein are the use of a compound of Formulas (I)-(XIX) in the manufacture of a medicament for treating a disease or disorder in a patient where modulation of 1,4,5,-trisphosphate 3 kinase B (ITPKB) is implicated.

Another aspect provided herein are methods for modulating B lymphocyte development and function in a system or subject, wherein the method includes administering to the system or the a therapeutically effective amount of a compound of Formulas (I)-(XIX), or pharmaceutically acceptable salts or pharmaceutical compositions thereof, wherein the compound modulates the kinase activity or cellular level of an ITPKB molecule thereby modulating B lymphocyte differentiation and function in the system or the subject. In certain embodiments of such methods, the methods include administering the compound to a cell or tissue system or to a human or an animal subject. In certain embodiments of such methods, the compound down-regulates the cellular level of the ITPKB molecule. In certain embodiments of such methods, the compound inhibits the kinase activity of the ITPKB molecule. In certain embodiments of such methods, the subject is human and the ITPKB molecule is human ITPKB.

Another aspect provided herein are methods for treating a disease or disorder where modulation of B lymphocyte development and function is implicated, comprising administering to a system or subject in need of such treatment an effective amount of a compound of Formulas (I)-(XIX), or pharmaceutically acceptable salts or pharmaceutical compositions thereof, thereby treating the disease or disorder. In certain embodiments of such methods, the system or subject is a cell or tissue system; or a human or animal subject. In certain embodiments of such methods, the disease or condition is an autoimmune disease. In certain embodiments of such methods, the autoimmune disease is rheumatoid arthritis, systemic lupus erythematosus, idiopathic thrombocytopenic purpura, hemolytic anemia, or psoriasis.

Another aspect provided herein are methods for treating a cell-proliferative condition, comprising administering to a system or subject in need of such treatment an effective amount of a compound of Formulas (I)-(XIX), or pharmaceutically acceptable salts or pharmaceutical compositions thereof; wherein the cell-proliferative condition is lymphoma. In certain embodiments of such methods, the lymphoma is B cell lymphoma.

Another aspect provided herein are compounds for use in a method of medical treatment, wherein the method of medical treatment is for treating a disease or disorder where modulation of B lymphocyte development and function is implicated. In certain embodiments, the disease or disorder is an autoimmune disease. In other embodiments, the autoimmune disease is rheumatoid arthritis, systemic lupus erythematosus, idiopathic thrombocytopenic purpura, hemolytic anemia, or psoriasis.

### DETAILED DESCRIPTION OF THE INVENTION

### Definitions

The terms “alkenyl” and “alkene,” as used herein, refers to a partially unsaturated branched or straight chain hydrocarbon having at least one carbon-carbon double bond. Atoms oriented about the double bond are in either the cis (Z) or trans (E) conformation. An alkenyl or alkene group can be optionally substituted. As used herein, the terms “C₂-C₃alkyenyl”, “C₂-C₄alkyenyl”, “C₂-C₅alkenyl”, “C₂-C₆alkenyl”, “C₂-C₇alkenyl”, and “C₂-C₈alkenyl” refer to an alkenyl group containing at least 2, and at most 3, 4, 5, 6, 7 or 8 carbon atoms, respectively. Non-limiting examples of alkenyl groups, as used herein, include ethenyl, propenyl, butenyl, pentenyl, hexenyl, heptenyl, octenyl, nonenyl, decenyl and the like. As used herein, the terms “C₂-C₃alkyene”, “C₂-C₄alkyene”, “C₂-C₅alkene”, “C₂-C₆alkene”, “C₂-C₇alkene”, and “C₂-C₈alkene” refer to an alkene group containing at least 2, and at most 3, 4, 5, 6, 7 or 8 carbon atoms, respectively. Non-limiting examples of alkene groups, as used herein, include ethene, propene, butene, pentene, hexene, heptene, octene, nonene, decene and the like.

The term “alkenylene,” as used herein, refers to a partially unsaturated branched or straight chain divalent hydrocarbon radical derived from an alkenyl group. An alkenylene group can be optionally substituted. As used herein, the terms “C₂-C₃alkenylene”, “C₂-C₄alkenylene”, “C₂-C₅alkenylene”, “C₂-C₆alkenylene”, “C₂-C₇alkenylene”, and “C₂-C₈alkenylene” refer to an alkenylene group containing at least 2, and at most 3, 4, 5, 6, 7 or 8 carbon atoms respectively. Non-limiting examples of alkenylene groups as used herein include, ethenylene, propenylene, butenylene, pentenylene, hexenylene, heptenylene, octenylene, nonenylene, decenylene and the like.

The term “alkyl,” as used herein, refers to a saturated branched or straight chain hydrocarbon. An alkyl group can be optionally substituted. As used herein, the terms “C₁-C₃alkyl”, “C₁-C₄alkyl”, “C₁-C₅alkyl”, “C₁-C₆alkyl”, “C₁-C₇alkyl” and “C₁-C₈alkyl” refer to an alkyl group containing at least 1, and at most 3, 4, 5, 6, 7 or 8 carbon atoms, respectively. Non-limiting examples of alkyl groups as used herein include methyl, ethyl, n-propyl, isopropyl, n-butyl, isobutyl, sec-butyl, t-butyl, n-pentyl, isopentyl, hexyl, heptyl, octyl, nonyl, decyl and the like.

The term “alkylene,” as used herein, refers to a saturated branched or straight chain divalent hydrocarbon radical derived from an alkyl group. An alkylene group can be optionally substituted. As used herein, the terms “C₁-C₃alkylene”, “C₁-C₄alkylene”, “C₁-C₅alkylene”, “C₁-C₆alkylene”, “C₁-C₇alkylene” and “C₁-C₈alkylene” refer to an alkylene group containing at least 1, and at most 3, 4, 5, 6, 7 or 8 carbon atoms respectively. Non-limiting examples of alkylene groups as used herein include, methylene, ethylene, n-propylene, isopropylene, n-butylene, isobutylene, sec-butylene, t-butylene, n-pentylene, isopentylene, hexylene and the like.

The term “alkynyl,” as used herein, refers to a partially unsaturated branched or straight chain hydrocarbon having at least one carbon-carbon triple bond. An alkynyl group can be optionally substituted. As used herein, the terms “C₂-C₃alkynyl”, “C₂-C₄alkynyl”, “C₂-C₅alkynyl”, “C₂-C₆alkynyl”, “C₂-C₇alkynyl”, and “C₂-C₈alkynyl” refer to an alkynyl group containing at least 2, and at most 3, 4, 5, 6, 7 or 8 carbon atoms, respectively. Non-limiting examples of alkynyl groups, as used herein, include ethynyl, propynyl, butynyl, pentynyl, hexynyl, heptynyl, octynyl, nonynyl, decynyl and the like.

The term “alkynylene,” as used herein, refers to a partially unsaturated branched or straight chain divalent hydrocarbon radical derived from an alkynyl group. An alkynylene group can be optionally substituted. As used herein, the terms “C₂-C₃alkynylene”, “C₂-C₄alkynylene”, “C₂-C₅alkynylene”, “C₂-C₆alkynylene”, “C₂-C₇alkynylene”, and “C₂-C₈alkynylene” refer to an alkynylene group containing at least 2, and at most 3, 4, 5, 6, 7 or 8 carbon atoms respectively. Non-limiting examples of alkynylene groups as used herein include, ethynylene, propynylene, butynylene, pentynylene, hexynylene, heptynylene, octynylene, nonynylene, decynylene and the like.

The term “alkoxy,” as used herein, refers to the group —ORₐ, where Rₐ is an alkyl group as defined herein. An alkoxy group can be optionally substituted. As used herein, the terms “C₁-C₃alkoxy”, “C₁-C₄alkoxy”, “C₁-C₅alkoxy”, “C₁-C₆alkoxy”, “C₁-C₇alkoxy” and “C₁-C₈alkoxy” refer to an alkoxy group wherein the alkyl moiety contains at least 1, and at most 3, 4, 5, 6, 7 or 8, carbon atoms. Non-limiting examples of alkoxy groups, as used herein, include methoxy, ethoxy, n-propoxy, isopropoxy, n-butyloxy, t-butyloxy, pentyloxy, hexyloxy, heptyloxy, octyloxy, nonyloxy, decyloxy and the like.

The term “aryl,” as used herein, refers to monocyclic, bicyclic, and tricyclic ring systems having a total of six to fourteen ring members, wherein at least one ring in the system is aromatic and wherein each ring in the system contains 3 to 7 ring members. An aryl group can be optionally substituted with one or more substituents. Non-limiting examples of aryl groups, as used herein, include phenyl, naphthyl, fluorenyl, indenyl, azulenyl, anthracenyl and the like.

The term “arylene,” as used means a divalent radical derived from an aryl group. An arylene group can be optionally substituted.

The term “cyano,” as used herein, refers to a —CN group.

The term “cycloalkyl,” as used herein, refers to a saturated or partially unsaturated, monocyclic, fused bicyclic, fused tricyclic or bridged polycyclic ring assembly. As used herein, the terms “C₃-C₅ cycloalkyl”, “C₃-C₆ cycloalkyl”, “C₃-C₇ cycloalkyl”, “C₃-C₈ cycloalkyl, “C₃-C₉ cycloalkyl and “C₃-C₁₀ cycloalkyl refer to a cycloalkyl group wherein the saturated or partially unsaturated, monocyclic, fused bicyclic or bridged polycyclic ring assembly contain at least 3, and at most 5, 6, 7, 8, 9 or 10, carbon atoms. A cycloalkyl group can be optionally substituted. Non-limiting examples of cycloalkyl groups, as used herein, include cyclopropyl, cyclobutyl, cyclopentyl, cyclohexyl, cycloheptyl, cyclooctyl, cyclononyl, cyclodecyl, cyclopentenyl, cyclohexenyl, decahydronaphthalenyl, 2,3,4,5,6,7-hexahydro-1H-indenyl and the like.

The term “halogen,” as used herein, refers to fluorine (F), chlorine (Cl), bromine (Br), or iodine (I).

The term “halo,” as used herein, refers to the halogen radicals: fluoro (—F), chloro (—Cl), bromo (—Br), and iodo (—I).

The terms “haloalkyl” or “halo-substituted alkyl,” as used herein, refers to an alkyl group as defined herein, substituted with one or more halogen groups, wherein the halogen groups are the same or different. A haloalkyl group can be optionally substituted. Non-limiting examples of such branched or straight chained haloalkyl groups, as used herein, include methyl, ethyl, propyl, isopropyl, isobutyl and n-butyl substituted with one or more halogen groups, wherein the halogen groups are the same or different, including, but not limited to, trifluoromethyl, pentafluoroethyl, and the like.

The terms “haloalkenyl” or “halo-substituted alkenyl,” as used herein, refers to an alkenyl group as defined herein, substituted with one or more halogen groups, wherein the halogen groups are the same or different. A haloalkenyl group can be optionally substituted. Non-limiting examples of such branched or straight chained haloalkenyl groups, as used herein, include ethenyl, propenyl, butenyl, pentenyl, hexenyl, heptenyl, octenyl, nonenyl, decenyl and the like substituted with one or more halogen groups, wherein the halogen groups are the same or different.

The terms “haloalkynyl” or “halo-substituted alkynyl,” as used herein, refers to an alkynyl group as defined above, substituted with one or more halogen groups, wherein the halogen groups are the same or different. A haloalkynyl group can be optionally substituted. Non-limiting examples of such branched or straight chained haloalkynyl groups, as used herein, include ethynyl, propynyl, butynyl, pentynyl, hexynyl, heptynyl, octynyl, nonynyl, decynyl, and the like substituted with one or more halogen groups, wherein the halogen groups are the same or different.

The term “haloalkoxy,” as used herein, refers to an alkoxy group as defined herein, substituted with one or more halogen groups, wherein the halogen groups are the same or different. A haloalkoxy group can be optionally substituted. Non-limiting examples of such branched or straight chained haloalkynyl groups, as used herein, include methoxy, ethoxy, n-propoxy, isopropoxy, n-butyloxy, t-butyloxy, pentyloxy, hexyloxy, heptyloxy, octyloxy, nonyloxy, decyloxy and the like, substituted with one or more halogen groups, wherein the halogen groups are the same or different.

The term “heteroalkyl,” as used herein, refers to an alkyl group as defined herein wherein one or more carbon atoms are independently replaced by one or more of oxygen, sulfur, nitrogen, or combinations thereof.

The term “heteroaryl,” as used herein, refers to monocyclic, bicyclic, and tricyclic ring systems having a total of five to fourteen ring members, wherein at least one ring in the system is aromatic, at least one ring in the system contains one or more heteroatoms selected from nitrogen, oxygen and sulfur, and wherein each ring in the system contains 3 to 7 ring members. A heteroaryl group can be optionally substituted with one or more substituents. Non-limiting examples of heteroaryl groups, as used herein, include benzofuranyl, benzofurazanyl, benzoxazolyl, benzopyranyl, benzthiazolyl, benzothienyl, benzazepinyl, benzimidazolyl, benzothiopyranyl, benzo[1,3]dioxole, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, pyridyl, pyridazinyl, pyrazinyl, pyrimidinyl, quinoxalinyl, quinolinyl, quinazolinyl, 4H-quinolizinyl, thiazolyl, thiadiazolyl, thienyl, triazinyl, triazolyl and tetrazolyl.

The term “heterocycloalkyl,” as used herein, refers to a cycloalkyl, as defined herein, wherein one or more of the ring carbons are replaced by a moiety selected from —O—, —N═, —NR—, —C(O)—, —S—, —S(O)— or —S(O)2-, wherein R is hydrogen, C₁-C₄alkyl or a nitrogen protecting group, with the proviso that the ring of said group does not contain two adjacent O or S atoms. A heterocycloalkyl group can be optionally substituted. Non-limiting examples of heterocycloalkyl groups, as used herein, include morpholino, pyrrolidinyl, pyrrolidinyl-2-one, piperazinyl, piperidinyl, piperidinylone, 1,4-dioxa-8-aza-spiro[4.5]dec-8-yl, 2H-pyrrolyl, 2-pyrrolinyl, 3-pyrrolinyl, 1,3-dioxolanyl, 2-imidazolinyl, imidazolidinyl, 2-pyrazolinyl, pyrazolidinyl, 1,4-dioxanyl, 1,4-dithianyl, thiomorpholinyl, azepanyl, hexahydro-1,4-diazepinyl, tetrahydrofuranyl, dihydrofuranyl, tetrahydrothienyl, tetrahydropyranyl, dihydropyranyl, tetrahydrothiopyranyl, thioxanyl, azetidinyl, oxetanyl, thietanyl, oxepanyl, thiepanyl, 1,2,3,6-tetrahydropyridinyl, 2H-pyranyl, 4H-pyranyl, dioxanyl, 1,3-dioxolanyl, dithianyl, dithiolanyl, dihydropyranyl, dihydrothienyl, dihydrofuranyl, imidazolinyl, imidazolidinyl, 3-azabicyclo[3.1.0]hexanyl, and 3-azabicyclo[4.1.0]heptanyl.

The term “heteroatom,” as used herein, refers to one or more of oxygen, sulfur, nitrogen, phosphorus, or silicon.

The term “hydroxyl,” as used herein, refers to the group —OH.

The term “hydroxyalkyl,” as used herein refers to an alkyl group as defined herein substituted with one or more hydroxyl group. Non-limiting examples of branched or straight chained “C₁-C₆ hydroxyalkyl groups as used herein include methyl, ethyl, propyl, isopropyl, isobutyl and n-butyl groups substituted with one or more hydroxyl groups.

The term “isocyanato,” as used herein, refers to a —N═C═O group.

The term “isothiocyanato,” as used herein, refers to a —N═C═S group.

The term “mercaptyl,” as used herein, refers to an (alkyl)S— group.

The term “optionally substituted,” as used herein, means that the referenced group may or may not be substituted with one or more additional group(s) individually and independently selected from alkyl, alkenyl, alkynyl, cycloalkyl, aryl, heteroaryl, heterocycloalkyl, hydroxyl, alkoxy, mercaptyl, cyano, halo, carbonyl, thiocarbonyl, isocyanato, thiocyanato, isothiocyanato, nitro, perhaloalkyl, perfluoroalkyl, and amino, including mono- and di-substituted amino groups, and the protected derivatives thereof. Non-limiting examples of optional substituents include, halo, —CN, ═O, —OR, —C(O)R, —C(O)OR, —OC(O)R, —OC(O)OR, —C(O)NHR, —C(O)NR₂, —OC(O)NHR, —OC(O)NR₂, —SR—, —S(O)R, —S(O)₂R, —NHR, —N(R)₂, —NHC(O)R, —NRC(O)R, —NHC(O)OR, —NRC(O)OR, S(O)₂NHR, —S(O)₂N(R)₂, —NHS(O)₂, —NRS(O)₂, —NHS(O)₂R, —NRS(O)₂R, C₁-C₈alkyl, C₁-C₈alkoxy, aryl, heteroaryl, cycloalkyl, heterocycloalkyl, halo-substituted C₁-C₈alkyl, halo-substituted C₁-C₈alkoxy, where each R is independently selected from H, halo, C₁-C₈alkyl, C₁-C₈alkoxy, aryl, heteroaryl, cycloalkyl, heterocycloalkyl, halo-substituted C₁-C₈alkyl, and halo-substituted C₁-C₈alkoxy. The placement and number of such substitutent groups is done in accordance with the well-understood valence limitations of each group, for example ═O is a suitable substituent for an alkyl group but not for an aryl group.

The term “solvate,” as used herein, refers to a complex of variable stoichiometry formed by a solute (by way of example, a compound of Formula (I), or a salt thereof, as described herein) and a solvent. Non-limiting examples of a solvent are water, acetone, methanol, ethanol and acetic acid.

The term “acceptable” with respect to a formulation, composition or ingredient, as used herein, means having no persistent detrimental effect on the general health of the subject being treated.

The term “administration” or “administering” of the subject compound means providing a compound of Formula (I), a pharmaceutically acceptable salt, a pharmaceutically acceptable solvate, or prodrug thereof to a subject in need of treatment.

The term “carrier,” as used herein, refers to chemical compounds or agents that facilitate the incorporation of a compound described herein into cells or tissues.

The terms “co-administration” or “combined administration” or the like as used herein are meant to encompass administration of the selected therapeutic agents to a single patient, and are intended to include treatment regimens in which the agents are not necessarily administered by the same route of administration or at the same time.

The term “dermatological disorder,” as used herein refers to a skin disorder. Such dermatological disorders include, but are not limited to, proliferative or inflammatory disorders of the skin such as, atopic dermatitis, bullous disorders, collagenoses, contact dermatitis eczema, Kawasaki Disease, rosacea, Sjogren-Larsso Syndrome, actinic keratosis, basal cell carcinoma and urticaria.

The term “diluent” as used herein, refers to chemical compounds that are used to dilute a compound described herein prior to delivery. Diluents can also be used to stabilize compounds described herein.

The terms “effective amount” or “therapeutically effective amount,” as used herein, refer to a sufficient amount of a compound described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “fibrosis” or “fibrosing disorder,” as used herein, refers to conditions that follow acute or chronic inflammation and are associated with the abnormal accumulation of cells and/or collagen and include but are not limited to fibrosis of individual organs or tissues such as the heart, kidney, joints, lung, or skin, and includes such disorders as idiopathic pulmonary fibrosis and cryptogenic fibrosing alveolitis.

The term “iatrogenic,” as used herein, means a condition, disorder, or disease created or worsened by medical or surgical therapy.

The term “immunologically effective amount,” as used herein, means that the administration of a sufficient amount to an individual, either in a single dose or as part of a series, that is effective for treatment or prevention of an immunological disease or disorder. This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The term “inflammatory disorders,” as used herein, refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which may be partial or complete, temporary or permanent). Inflammation takes many forms and includes, but is not limited to, inflammation that is one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporarl arteritis); joints (arthritis: crystalline, osteo-, psoriatic, reactive, rheumatoid, Reiter's); gastrointestinal tract; skin (dermatitis); or multiple organs and tissues (systemic lupus erythematosus).

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an inhibitor or an enhancer.

The term “pharmaceutically acceptable,” as used herein, refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “pharmaceutically acceptable salt,” as used herein, refers to a formulation of a compound that does not cause significant irritation to an organism to which it is administered and does not abrogate the biological activity and properties of the compounds described herein.

The terms “combination” or “pharmaceutical combination,” as used herein mean a product that results from the mixing or combining of more than one active ingredient and includes both fixed and non-fixed combinations of the active ingredients. The term “fixed combination” means that the active ingredients, by way of example, a compound of Formula (I) and an additional therapeutic agent, are both administered to a patient simultaneously in the form of a single entity or dosage. The term “non-fixed combination” means that the active ingredients, by way of example, a compound of Formula (I) and an additional therapeutic agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

The terms “composition” or “pharmaceutical composition,” as used herein, refers to a mixture of at least one compound of Formula (I) described herein with other chemical components, such as carriers, stabilizers, diluents, dispersing agents, suspending agents, thickening agents, and/or excipients.

The term “prodrug,” as used herein, refers to an agent that is converted into the parent drug in vivo. A non-limiting example of a prodrug of the compounds described herein is a compound described herein administered as an ester which is then metabolically hydrolyzed to a carboxylic acid, the active entity, once inside the cell. A further example of a prodrug is a short peptide bonded to an acid group where the peptide is metabolized to reveal the active moiety.

The term “respiratory disease,” as used herein, refers to diseases affecting the organs that are involved in breathing, such as the nose, throat, larynx, trachea, bronchi, and lungs. Respiratory diseases include, but are not limited to, asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia.

The term “subject” or “patient,” as used herein, encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish and the like.

The term “therapeutically effective amount,” as used herein, refers to any amount of a compound which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The terms “treat,” “treating” or “treatment,” as used herein, refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

Other objects, features and advantages of the methods, compositions and combinations described herein will become apparent from the following detailed description. It should be understood, however, that the detailed description and the specific examples, while indicating specific embodiments, are given by way of illustration only.

### Compounds

Provided herein are compounds, pharmaceutically acceptable salts, solvates, N-oxides, prodrugs and isomers thereof that are modulators of IPTKB kinase activity. In certain embodiments such compounds, pharmaceutically acceptable salts, solvates, N-oxides, prodrugs and isomers thereof are inhibitors of IPTKB kinase activity. Also provided herein are compounds, pharmaceutically acceptable salts, solvates, N-oxides, prodrugs and isomers thereof that are modulators of the cellular level/cellular concentration of IPTKB kinase, wherein such compounds, pharmaceutically acceptable salts, solvates, N-oxides, prodrugs and isomers thereof modulate the ITPKb gene expressing the ITPKB kinase. In certain embodiments, such genes are down regulated thereby down regulating the cellular level/cellular concentration of IPTKB kinase.

Further provided herein are compounds, pharmaceutically acceptable salts, solvates, N-oxides, prodrugs and isomers thereof, and pharmaceutical compositions containing such pharmaceutically acceptable salts, solvates, N-oxides, prodrugs and isomers thereof, for the treatment and/or prevention of diseases and/or disorders in which aberrant, abnormal or deregulated activity of IPTKB contributes to the pathology and/or symptomology of such diseases and/or disorders. In certain embodiments, such diseases and/or disorders are associated with or mediated by abnormal B-cell proliferation, differentiation and activation. Such diseases and/or disorders include, but are not limited to, B-cell lymphoma, chronic transplant rejection, immune-mediated disease, autoimmune mediated diseases, and anaphylaxis and many complement mediated diseases. Such immune mediated disorders include, but are not limited to, allergy and psoriasis. Such autoimmune mediated disorders include, but are not limited to, rheumatoid arthritis (RA), systematic lupus erythematosus (SLE), hemolytic anemia, lupus, primary binary cirrhosis (PBC) and idiopathic thrombocytopenic purpura (ITP). Such allergy disorders include, but are not limited to, respiratory diseases and dermatolgical disorders. Respiratory diseases include but are not limited to, asthma, rhinitis, COPD, asthma, bronchial asthma, allergic asthma, intrinsic asthma, extrinsic asthma, exercise-induced asthma, drug-induced asthma (including aspirin and NSAID-induced) and dust-induced asthma, chronic obstructive pulmonary disease (COPD); bronchitis, acute and chronic rhinitis including rhinitis medicamentosa, and vasomotor rhinitis, and perennial and seasonal allergic rhinitis including rhinitis nervosa (hay fever). Dermatological diseases and/or disorders include, but are not limited to, dermatitis and eczema such as, by way of example only, atopic dermatitis, seborrhoeic dermatitis (Dandruff, Cradle cap), diaper rash, urushiol-induced contact dermatitis, contact dermatitis, erythroderma, lichen simplex chronicus, prurigo nodularis, itch, pruritus ani, nummular dermatitis, dyshidrosis and pityriasis alba.

Further provided herein are methods for the treatment and/or prevention of diseases and/or disorders in which aberrant, abnormal or deregulated activity of IPTKB contributes to the pathology and/or symptomology of such diseases and/or disorders. In certain embodiments, such diseases and/or disorders are associated with or mediated by abnormal B-cell proliferation, differentiation and activation. Such diseases and/or disorders include, but are not limited to, B-cell lymphoma, chronic transplant rejection, immune-mediated disease, autoimmune mediated diseases, and anaphylaxis and many complement mediated diseases. Such immune mediated disorders include, but are not limited to, allergy and psoriasis. Such autoimmune mediated disorders include, but are not limited to, rheumatoid arthritis (RA), systematic lupus erythematosus (SLE), hemolytic anemia, lupus, primary binary cirrhosis (PBC) and idiopathic thrombocytopenic purpura (ITP). Such allergy disorders include, but are not limited to, respiratory diseases and dermatolgical disorders. Respiratory diseases include but are not limited to, asthma, rhinitis, COPD, asthma, bronchial asthma, allergic asthma, intrinsic asthma, extrinsic asthma, exercise-induced asthma, drug-induced asthma (including aspirin and NSAID-induced) and dust-induced asthma, chronic obstructive pulmonary disease (COPD); bronchitis, acute and chronic rhinitis including rhinitis medicamentosa, and vasomotor rhinitis, and perennial and seasonal allergic rhinitis including rhinitis nervosa (hay fever). Dermatological diseases and/or disorders include, but are not limited to, dermatitis and eczema such as, by way of example only, atopic dermatitis, seborrhoeic dermatitis (Dandruff, Cradle cap), diaper rash, urushiol-induced contact dermatitis, contact dermatitis, erythroderma, lichen simplex chronicus, prurigo nodularis, itch, pruritus ani, nummular dermatitis, dyshidrosis and pityriasis alba.

In certain embodiments, the compounds, pharmaceutically acceptable salts, solvates, N-oxides, prodrugs and isomers thereof, and pharmaceutical compositions provided herein are inhibitors of ITPKB kinase activity and are thereby inhibitors of B-cell proliferation, differentiation and activation.

In certain embodiments, the compounds, pharmaceutically acceptable salts, solvates, N-oxides, prodrugs and isomers thereof, and pharmaceutical compositions provided herein are used as immunosuppressant agents to treat and/or prevent rheumatoid arthritis (RA), multiple sclerosis (MS), systemic lupus erythematosus (SLE), immune thrombocytopenic purpura (ITP), hemolytic anemia and transplant rejection.

The aforementioned compounds and pharmaceutically acceptable salts, solvates, N-oxides, prodrugs and isomers thereof, are compounds having structures according to Formula (I), wherein Formula (I) is

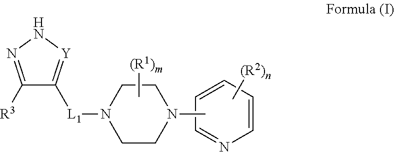

wherein:
L₁ is —(CR¹¹R¹²)ₚ—, —C(O)—, or —S(O)₂—;
L₂ is —C(O)—, —C(O)NR⁵— or —NR⁵C(O;
Y is N or CR⁴;
each R¹ is independently selected from —C(O)R⁹, C₁-C₆alkyl, C₁-C₆heteroalkyl, C₁-C₆haloalkyl, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, aryl, heteroaryl, C₃-C₈cycloalkyl, and C₃-C₁₀heterocycloalkyl groups of R¹ are each optionally substituted with 1 to 3 substituents independently selected from halogen, —CN, C₁-C₆alkyl, C₁-C₆haloalkyl, C₁-C₆alkoxy, C₁-C₆haloalkoxyl, C₃-C₈cycloalkyl, C₃-C₁₀heterocycloalkyl, —OR⁹, —C(O)R⁹, —OC(O)R⁹, —C(O)OR⁹, —N(R⁶R⁷⁾, —C(O)N(R⁶R⁷), —S(O)₂R⁹, —S(O)₂N(R⁶R⁷), and —NR⁷S(O)₂R⁹;
or two R₁ groups are each independently C₁-C₄alkyl and form a C₁-C₄alkyl bridge, or two R₁ groups are each independently C₁-C₄alkyl and taken together with the C atom to which they are attached form an optionally substituted C₃-C₈cycloalkyl;
each R² is independently selected from halogen, —CN, —OR⁹, —C(O)R⁹, —C(O)N(R⁶R⁷), C₁-C₆alkyl, C₁-C₆heteroalkyl, C₁-C₆haloalkyl, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, aryl, heteroaryl, C₃-C₈cycloalkyl, and C₃-C₁₀heterocycloalkyl groups of R² are each optionally substituted with 1 to 3 substituents independently selected from halogen, —CN, C₁-C₆alkyl, C₁-C₆haloalkyl, C₁-C₆ alkoxy, C₁-C₆haloalkoxyl, C₃-C₈cycloalkyl, C₃-C₁₀heterocycloalkyl —OR⁹, —C(O)R⁹, —OC(O)9-C(O)OR⁹, —N(R⁶R⁷), —C(O)N(R⁶R⁷), —S(O)₂R⁹, —S(O)₂N(R⁶R⁷), and —NR⁷S(O)₂R⁹;
when Y is N then R³ is selected from L₂-R¹⁰, C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, C₃-C₈cycloalkyl, C₃-C₁₀heterocycloalkyl, C₆₋₁₀aryl and C₂-C₉heteroaryl, wherein the C₁-C₆alkyl, C₁-C₆heteroalkyl, C₁-C₆haloalkyl, C₁-C₆alkoxy, C₁-C₆haloalkoxy, C₂-C₉heteroaryl, C₃-C₈cycloalkyl, aryl and C₃-C₁₀heterocycloalkyl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —NR⁶C(O)R⁷, —C(O)N(R⁶R⁷), —S(O)₂R⁹, —S(O)₂N(R⁶R⁷) and —NR⁷S(O)₂R⁹;
when Y is CR⁴ then R³ is selected from L₂-R¹⁰, C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, C₃-C₈cycloalkyl, C₃-C₁₀heterocycloalkyl and C₂-C₉heteroaryl, provided that R³ is not a six-membered heteroaryl containing 1 to 3 N atoms, and wherein the C₁-C₆alkyl, C₁-C₆heteroalkyl, C₁-C₆haloalkyl, C₁-C₆alkoxy, C₁-C₆haloalkoxy, C₂-C₉heteroaryl, C₃-C₈cycloalkyl and C₃-C₁₀heterocycloalkyl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —NR⁶C(O)R⁷, —C(O)N(R⁶R⁷), —S(O)₂R⁹, —S(O)₂N(R⁶R⁷) and —NR⁷S(O)₂R⁹;
R⁴ is selected from H, —C(O)OR⁹, —C(O)R⁹, —C(O)N(R⁶R⁷), —N(R⁶R⁷), —NR⁶C(O)R⁷, —(CH₂)ₙOR⁷, C₁-C₆alkyl, C₁-C₆heteroalkyl, C₁-C₆haloalkyl, C₂^{-C}₈alkene, C₂-C₈alkyne, C₁-C₆alkoxy, C₁-C₆haloalkoxy, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₁₀heterocycloalkyl groups of R⁵ are each optionally substituted with 1 to 3 substituents independently selected from halogen, —CN, —R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷), and —NR⁷S(O)₂R⁹;
R⁵, R⁶ and R⁷ are each independently selected from H, C₁-C₆alkyl, C₁-C₆haloalkyl, C₁-C₆alkoxy, C₃-C₈cycloalkyl, C₃-C₁₀heterocycloalkyl, C₁-C₆haloalkyl, C₁-C₆haloalkoxy, aryl and heteroaryl, wherein the C₁-C₆alkyl, C₁-C₆haloalkyl, C₁-C₆alkoxy, C₃-C₈cycloalkyl, C₃-C₁₀heterocycloalkyl, C₁-C₆haloalkyl, C₁-C₆haloalkoxy, aryl and heteroaryl of R⁵, R⁶ and R⁷ are each optionally substituted with 1 to 3 substituents independently selected from halogen, —CN, —R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷), and —NR⁷S(O)₂R⁹,
or R⁶ and R⁷ are each independently C₁-C₄alkyl and taken together with the C atom to which they are attached form a C₃-C₈cycloalkyl;
R⁸ is selected from H, CN, —OR⁹, —C(O)R⁹, —C(O)OR⁹, —C(O)N(R⁶R⁷), —C(═NH)N(R⁶R⁷), C₁-C₆alkyl, C₁-C₆haloalkyl, C₁-C₆alkoxy, C₁-C₆haloalkoxy, C₃-C₈cycloalkyl, and C₃-C₁₀heterocycloalkyl;
R⁹ is selected from H, C₁-C₆alkyl, C₁-C₆haloalkyl, C₁-C₆alkoxy, C₃-C₈cycloalkyl, C₃-C₁₀heterocycloalkyl, C₁-C₆haloalkyl and C₁-C₆haloalkoxy;
R¹⁰ is selected from C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, C₁-C₆alkoxy, C₁-C₆haloalkoxy, aryl, heteroaryl, C₃-C₈cycloalkyl, and C₃-C₁₀heterocycloalkyl groups of R¹¹ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁷, —S(O)₂N(R⁶R⁷) and —NR⁷S(O)₂R⁹;
R¹¹ and R¹² are each independently selected from H, C₁-C₄alkyl, C₁-C₄heteroalkyl, C₁-C₄haloalkyl, C₁-C₄alkoxy and C₁-C₄haloalkoxy;
or R¹¹ and R¹² are each independently C₁-C₄alkyl and taken together with the C atom to which they are attached form a C₃-C₈cycloalkyl;
m is, independently at each occurrence, 0, 1, 2, 3 or 4;
n is, independently at each occurrence, 0, 1, 2, 3 or 4, and
p is, independently at each occurrence, 1, 2, 3 or 4.

In certain embodiments, such compounds of Formula (I), have a structure according to Formula (II):

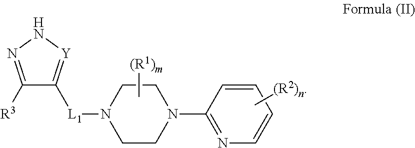

In certain embodiments, n is 0, 1 or 2, while in other embodiments, such compounds of Formulas (I)-(II), have a structure according to Formula (III):

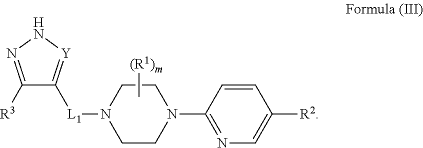

In certain embodiments, m is 0, 1 or 2, while in other embodiments, such compounds of Formulas (I)-(III), have a structure according to Formula (IV) or Formula (V):

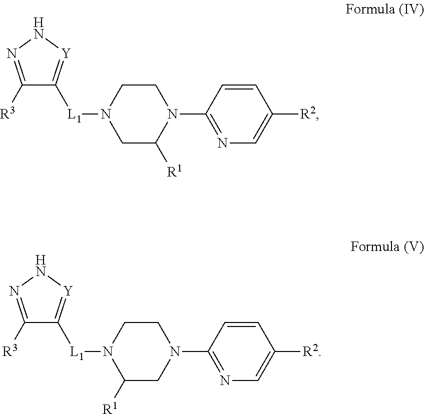

In certain embodiments of such compounds of Formulas (I)-(V), L₁ is —(CR¹¹R¹²)ₚ—. In other embodiments of such compounds of Formulas (I)-(V), R¹¹ and R¹² are each independently selected from H and C₁-C₄alkyl.

In other embodiments of such compounds of Formulas (I)-(V), L₁ is —(CH₂)— and such compounds have a structure according to Formula (VI) or Formula (VII):

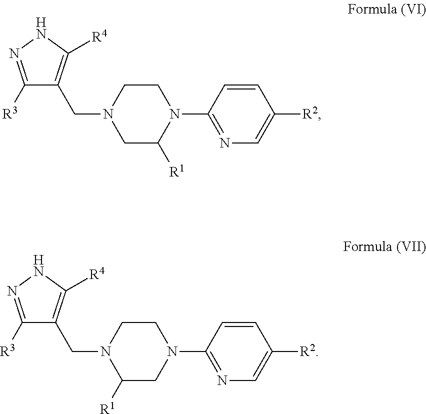

In other embodiments of such compounds of Formulas (I)-(VII), R⁴ is H, and such compounds have a structure according to Formula (VIII) or Formula (IX):

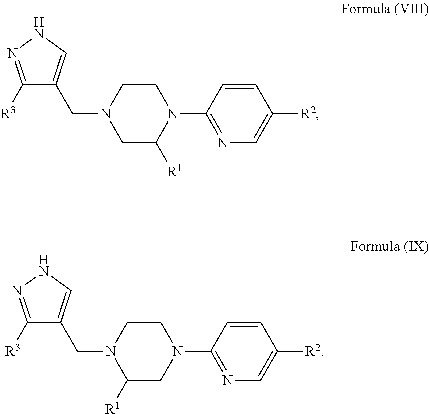

In other embodiments of such compounds of Formulas (I)-(V), L₁ is —(CH₂)— and such compounds have a structure according to Formula (X) or Formula (XI):

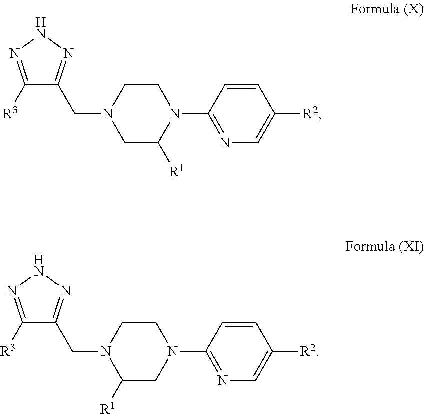

In other embodiments of such compounds of Formulas (I)-(XI), R¹ is C₁-C₆alkyl or C₁-C₆haloalkyl, while in other embodiments of such compounds of Formulas (I)-(XI), R² is C₁-C₆alkyl or C₁-C₆haloalkyl. In certain embodiments of such compounds of Formulas (I)-(XI), R¹ is methyl, ethyl, trifluoromethyl, difluoromethyl or fluoromethyl, while in other embodiments of such compounds of Formulas (I)-(XI), R² is methyl, ethyl, trifluoromethyl, difluoromethyl or fluoromethyl.

In other embodiments, such compounds of Formulas (I) have a structure according to Formula (XII), Formula (XIII), Formula (XIV) or Formula (XV):

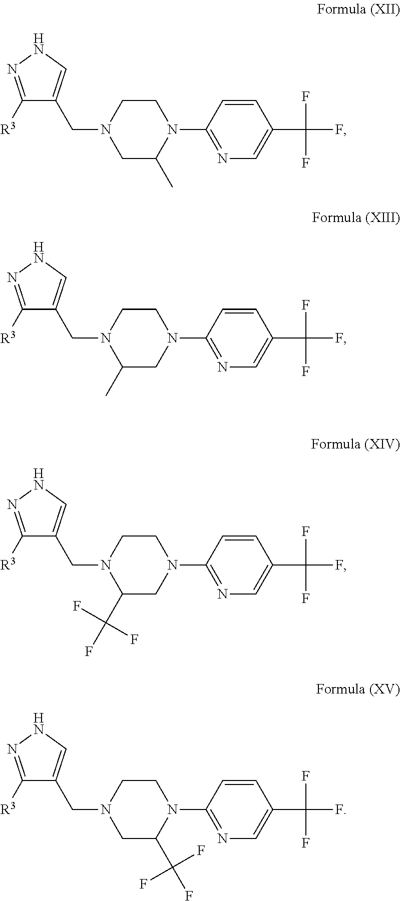

In other embodiments, such compounds of Formulas (I) have a structure according to Formula (XVI), Formula (XVII), Formula (XVIII) or Formula (XIX):

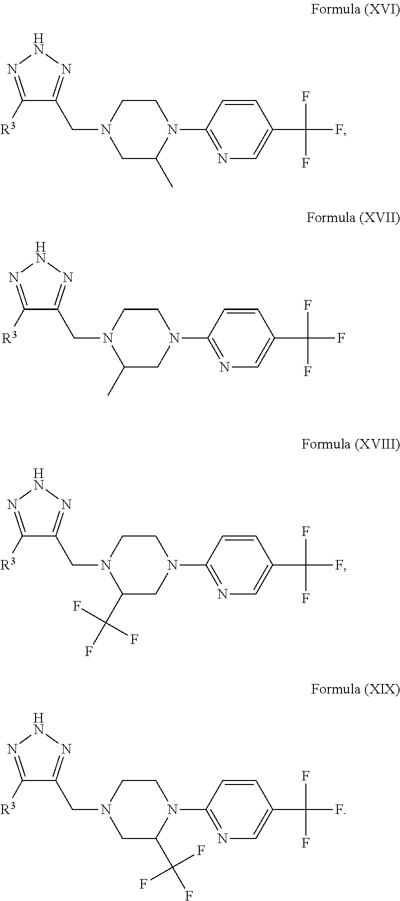

In certain embodiments of such compounds of Formulas (I)-(XIX) when Y is CR⁴, then R³ is C₃-C₁₀heterocycloalkyl or C₂-C₉heteroaryl, wherein the C₃-C₁₀heterocycloalkyl and C₂-C₉heteroaryl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷) and —NR⁷S(O)₂R⁹ and provided that R³ is not a six-membered heteroaryl containing 1 to 3 N atoms. In certain embodiments of such compounds of Formulas (I)-(XIX) when Y is CR⁴, then the C₃-C₁₀heterocycloalkyl and C₂-C₉heteroaryl groups of R³ are substituted with R⁸. In certain embodiments of such compounds of Formulas (I)-(XIX) when Y is CR⁴, the C₂-C₉heteroaryl is selected from benzofuranyl, benzofurazanyl, benzoxazolyl, benzopyranyl, benzthiazolyl, benzothienyl, benzazepinyl, benzimidazolyl, benzothiopyranyl, benzo[1,3]dioxole, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, quinoxalinyl, quinolinyl, quinazolinyl, 4H-quinolizinyl, thiazolyl, thiadiazolyl, thienyl, triazolyl and tetrazolyl.

In certain embodiments of such compounds of Formulas (I)-(XIX) when Y is N, then R³ is aryl, C₃-C₁₀heterocycloalkyl or C₂-C₉heteroaryl, wherein the aryl, C₃-C₁₀heterocycloalkyl and C₂-C₉heteroaryl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷) and —NR⁷S(O)₂R⁹. In certain embodiments of such compounds of Formulas (I)-(XIX) when Y is N, then the C₃-C₁₀heterocycloalkyl and C₂-C₉heteroaryl groups of R³ are substituted with R⁸. In certain embodiments of such compounds of Formulas (I)-(XIX) when Y is N, the C₂-C₉heteroaryl is selected from benzofuranyl, benzofurazanyl, benzoxazolyl, benzopyranyl, benzthiazolyl, benzothienyl, benzazepinyl, benzimidazolyl, benzothiopyranyl, benzo[1,3]dioxole, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, pyridazinyl, pyrazinyl, pyrimidinyl, quinoxalinyl, quinolinyl, quinazolinyl, 4H-quinolizinyl, thiazolyl, thiadiazolyl, thienyl, triazinyl, triazolyl and tetrazolyl.

In certain embodiments of such compounds of Formulas (I)-(XIX), R³ is L₂-R¹⁰, while in other embodiments L₂ is selected from C₁-C₆alkenylene, —C(O)— and —C(O)NR⁵, and in other embodiments R¹⁰ is selected from aryl, heteroaryl and C₃-C₁₀heterocycloalkyl, wherein the aryl, heteroaryl and C₃-C₁₀heterocycloalkyl groups of R¹⁰ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁷, —S(O)₂N(R⁶R⁷) and —NR⁷S(O)₂R⁹. In still other embodiments, of such compounds of Formulas (I)-(XIX), R¹⁰ is selected from aryl, heteroaryl and C3-C10heterocycloalkyl, wherein the aryl, heteroaryl and C₃-C₁₀heterocycloalkyl groups of R¹⁰ are substituted with R⁸.

In certain embodiments of such compounds of Formulas (I)-(XIX), R⁸ is selected from CN, —OR⁹, —C(O)R⁹⁰, —C(O)OR⁹, —C(O)N(R⁶R⁷), and —C(═NH)N(R⁶R⁷).

In certain embodiments of such compounds of Formulas (I)-(XIX), R³ is selected from isoquinoline, 2-oxo-1,2-dihydropyridine-4-carbonitrile, thiophene, pyrrole, 1H-pyrrole-3-carbonitrile, phenyl, benzimidazole, 5-phenyl-1H-imidazole, 5-fluoro-1H-benzo[d]imidazole, 4,5,6,7-tetrahydro-1H-benzo[d]imidazole, imidazole, 5-methyl-1H-imidazole, 4,5-dimethyl-1H-imidazol, 1H-imidazol4,5-c]pyridine, 4-(trifluoromethyl)-1H-imidazole, 1H-benzo[d]imidazole-5-carbonitrile, 1H-imidazole-4-carbonitrile, 1H-pyrrole-3-carboxamide, 1H-pyrrole-2-carboxamide, 1H-pyrrole-2-carbonitrile, furan-2-carboxylic acid, furan-2-carboxamide, furan-3-carboxamide, methyl furan-2-carboxylate, N-methyl-1H-pyrrole-3-carboxamide, 1H-pyrrolo[2,3-b]pyridine, N,N-dimethyl-1H-pyrrole-3-carboxamide, N-(2-hydroxypropyl)-1H-pyrrole-3-carboxamide, (S)—N-(1-hydroxypropan-2-yl)-1H-pyrrole-3-carboxamide, 1H-indole, N-(2-hydroxyethyl)-1H-pyrrole-3-carboxamide, 1,2,3,6-tetrahydropyridine, 5,6-dihydropyridine-1(2H)-carbaldehyde, 1-(5,6-dihydropyridin-1(2H)-yl)ethanone, 1-(5,6-dihydropyridin-1(2H)-yl)-3-hydroxypropan-1-one, piperidine, 1-(piperidin-1-yl)ethanone, piperidine-1-carbaldehyde, 1H-imidazole-4-carboximidamide and 1H-imidazole-4-carboxamide.In other embodiments of such compounds of Formulas (I)-(XIX), R⁵ is H or C₁-C₆alkyl.

In certain embodiments of such compounds of Formulas (I)-(XIX), L₂ is —C(O)NR⁵— and R¹⁰ is selected from 2H-benzo[b][1,4]oxazin-3(4H)-one, 1-phenyl-1H-imidazole, N-(5-methylisoxazol-3-yl)benzenesulfonamide, 1H-indole, 1H-imidazole-5-carbonitrile, 3-(furan-2-yl)-1H-pyrazole, N,N-dimethyl-2-(3-methyl-1H-pyrazol-1-yl)ethanamine and 1H-pyrazole-4-carbonitrile.

In certain embodiments of such compounds of Formulas (I)-(XIX), The compound of any of claims 1-24, wherein L₂ is —C(O)— and R¹⁰ is selected from azetidin-3-ol, pyrrolidin-3-ol and piperidin-4-ol.

In certain embodiments of such compounds of Formulas (I)-(XIX), R⁶ is H or C₁-C₆alkyl, while in other embodiments of such compounds of Formulas (I)-(XIX), R⁷ is H or C₁-C₆alkyl. In certain embodiments of such compounds of Formulas (I)-(XIX), R⁹ is H or C₁-C₆alkyl.

In certain embodiments, the compounds of Formula (I) are (R)-4-((3-methyl-4-(5-(trifluoromethyl)pyridin-2-yl)piperazin-1-yl)methyl)-N-(pyridin-4-yl)-1H-pyrazole-3-carboxamide; (R)-5-(4-((3-methyl-4-(5-(trifluoromethyl)pyridin-2-yl)piperazin-1-yl)methyl)-1H-pyrazol-3-yl)-1H-pyrrole-3-carbonitrile; (R)-5-(4-((3-methyl-4-(5-(trifluoromethyl)pyridin-2-yl)piperazin-1-yl)methyl)-1H-pyrazol-3-yl)-1H-pyrrole-3-carboxamide; (R)—N-methyl-5-(4-((3-methyl-4-(5-(trifluoromethyl)pyridin-2-yl)piperazin-1-yl)methyl)-1H-pyrazol-3-yl)-1H-pyrrole-3-carboxamide; (R)-5-(4-((3-methyl-4-(5-(trifluoromethyl)pyridin-2-yl)piperazin-1-yl)methyl)-1H-pyrazol-3-yl)-1H-pyrrole-2-carbonitrile; (R)-5-(4-((3-methyl-4-(5-(trifluoromethyl)pyridin-2-yl)piperazin-1-yl)methyl)-1H-pyrazol-3-yl)-1H-pyrrole-2-carboxamide; (R)-4-(4-((3-methyl-4-(5-(trifluoromethyl)pyridin-2-yl)piperazin-1-yl)methyl)-1H-pyrazol-3-yl)-1H-pyrrole-2-carbonitrile; (R)-4-(4-((3-methyl-4-(5-(trifluoromethyl)pyridin-2-yl)piperazin-1-yl)methyl)-1H-pyrazol-3-yl)-1H-pyrrole-2-carboxamide; (R)-2-(4-((3-methyl-4-(5-(trifluoromethyl)pyridin-2-yl)piperazin-1-yl)methyl)-1H-pyrazol-3-yl)-1H-indole; (R)-2-methyl-4-((3-(1,2,3,6-tetrahydropyridin-4-yl)-1H-pyrazol-4-yl)methyl)-1-(5-(trifluoromethyl)pyridin-2-yl)piperazine; (R)-4-(4-((3-methyl-4-(5-(trifluoromethyl)pyridin-2-yl)piperazin-1-yl)methyl)-1H-pyrazol-3-yl)-5,6-dihydropyridine-1(2H)-carbaldehyde; (R)-2-methyl-4-((3-(4-(trifluoromethyl)-1H-imidazol-2-yl)-1H-pyrazol-4-yl)methyl)-1-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-1H-imidazole-4-carboximidamide; (R)-2-(4-4-((3-methyl-4-(5-(trifluoromethyl)pyridin-2-yl)piperazin-1-yl)methyl)-1H-pyrazol-3-yl)-1H-imidazo[4,5-c]pyridine; (R,Z)-5-((4-((3-methyl-4-(5-(trifluoromethyl)pyridin-2-yl)piperazin-1-yl)methyl)-1H-pyrazol-3-yl)methylene)imidazolidine-2,4-dione; (R)-6-(4-((3-methyl-4-(5-(trifluoromethyl)pyridin-2-yl)piperazin-1-yl)methyl)-1H-pyrazol-3-yl)isoquinoline; (R)-6-(4-((3-methyl-4-(5-(trifluoromethyl)pyridin-2-yl)piperazin-1-yl)methyl)-1H-pyrazol-3-yl)-2-oxo-1,2-dihydropyridine-3-carbonitrile; 1-((3-(thiophen-2-yl)-1H-pyrazol-4-yl)methyl)-4-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-1H-benzo[d]imidazole; (R)-2-methyl-4-((3-(5-methyl-1H-imidazol-2-yl)-1H-pyrazol-4-yl)methyl)-1-(5-(trifluoromethyl)pyridin-2-yl)piperazine; (R)-4-((3-(4,5-dimethyl-1H-imidazol-2-yl)-1H-pyrazol-4-yl)methyl)-2-methyl-1-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-4,5,6,7-tetrahydro-1H-benzo[d]imidazole; (R)-5-fluoro-2-(4-((3-methyl-4-(5-(trifluoromethyl)pyridin-2-yl)piperazin-1-yl)methyl)-1H-pyrazol-3-yl)-1H-benzo[d]imidazole; (R)-2-(4-((3-methyl-4-(5-(trifluoromethyl)pyridin-2-yl)piperazin-1-yl)methyl)-1H-pyrazol-3-yl)-1H-benzo[d]imidazole-5-carbonitrile; (S)-5-(4-((3-(trifluoromethyl)-4-(5-(trifluoromethyl)pyridin-2-yl)piperazin-1-yl)methyl)-1H-pyrazol-3-yl)-1H-pyrrole-3-carbonitrile; (R)—N-(5-cyano-1H-imidazol-4-yl)-4-((3-methyl-4-(5-(trifluoromethyl)pyridin-2-yl)piperazin-1-yl)methyl)-1H-pyrazole-3-carboxamide; (R)—N-(3-(furan-2-yl)-1H-pyrazol-5-yl)-4-((3-methyl-4-(5-(trifluoromethyl)pyridin-2-yl)piperazin-1-yl)methyl)-1H-pyrazole-3-carboxamide; (R)—N-(1-(2-(dimethylamino)ethyl)-3-methyl-1H-pyrazol-5-yl)-4-((3-methyl-4-(5-(trifluoromethyl)pyridin-2-yl)piperazin-1-yl)methyl)-1H-pyrazole-3-carboxamide; (R)—N-(4-cyano-1H-pyrazol-3-yl)-4-((3-methyl-4-(5-(trifluoromethyl)pyridin-2-yl)piperazin-1-yl)methyl)-1H-pyrazole-3-carboxamide; (R)-(3-hydroxyazetidin-1-yl)(4-((3-methyl-4-(5-(trifluoromethyl)pyridin-2-yl)piperazin-1-yl)methyl)-1H-pyrazol-3-yl)methanone; (3-hydroxypyrrolidin-1-yl)(4-(((R)-3-methyl-4-(5-(trifluoromethyl)pyridin-2-yl)piperazin-1-yl)methyl)-1H-pyrazol-3-yl)methanone; (R)-(4-hydroxypiperidin-1-yl)(4-((3-methyl-4-(5-(trifluoromethyl)pyridin-2-yl)piperazin-1-yl)methyl)-1H-pyrazol-3-yl)methanone; (R)-methyl 5-(4-((3-methyl-4-(5-(trifluoromethyl)pyridin-2-yl)piperazin-1-yl)methyl)-1H-pyrazol-3-yl)furan-2-carboxylate; (R)-5-(4-((3-methyl-4-(5-(trifluoromethyl)pyridin-2-yl)piperazin-1-yl)methyl)-1H-pyrazol-3-yl)furan-2-carboxylic acid; (R)-5-(4-((3-methyl-4-(5-(trifluoromethyl)pyridin-2-yl)piperazin-1-yl)methyl)-1H-pyrazol-3-yl)furan-2-carboxamide; (R)-4-(4-((3-methyl-4-(5-(trifluoromethyl)pyridin-2-yl)piperazin-1-yl)methyl)-1H-pyrazol-3-yl)-1H-pyrrolo[2,3-b]pyridine; (R)—N,N-dimethyl-5-(4-((3-methyl-4-(5-(trifluoromethyl)pyridin-2-yl)piperazin-1-yl)methyl)-1H-pyrazol-3-yl)-1H-pyrrole-3-carboxamide; (R)—N-(2-hydroxyethyl)-5-(4-((3-methyl-4-(5-(trifluoromethyl)pyridin-2-yl)piperazin-1-yl)methyl)-1H-pyrazol-3-yl)-1H-pyrrole-3-carboxamide; N-(2-hydroxypropyl)-5-(4-(((R)-3-methyl-4-(5-(trifluoromethyl)pyridin-2-yl)piperazin-1-yl)methyl)-1H-pyrazol-3-yl)-1H-pyrrole-3-carboxamide; (R)-3-(4-((3-methyl-4-(5-(trifluoromethyl)pyridin-2-yl)piperazin-1-yl)methyl)-1H-pyrazol-3-yl)-1H-pyrrolo[2,3-b]pyridine; N—((S)-1-hydroxypropan-2-yl)-5-(4-(((R)-3-methyl-4-(5-(trifluoromethyl)pyridin-2-yl)piperazin-1-yl)methyl)-1H-pyrazol-3-yl)-1H-pyrrole-3-carboxamide; (R,Z)-5-((4-((3-methyl-4-(5-(trifluoromethyl)pyridin-2-yl)piperazin-1-yl)methyl)-1H-pyrazol-3-yl)methylene)-2-thioxoimidazolidin-4-one; (R)-1-(4-(4-((3-methyl-4-(5-(trifluoromethyl)pyridin-2-yl)piperazin-1-yl)methyl)-1H-pyrazol-3-yl)-5,6-dihydropyridin-1(2H)-yl)ethanone; (R)-2-methyl-4-((3-(piperidin-4-yl)-1H-pyrazol-4-yl)methyl)-1-(5-(trifluoromethyl)pyridin-2-yl)piperazine; (R)-4-(4-((3-methyl-4-(5-(trifluoromethyl)pyridin-2-yl)piperazin-1-yl)methyl)-1H-pyrazol-3-yl)piperidine-1-carbaldehyde; (R,Z)-2-imino-5-((4-((3-methyl-4-(5-(trifluoromethyl)pyridin-2-yl)piperazin-1-yl)methyl)-1H-pyrazol-3-yl)methylene)imidazolidin-4-one; (S)-5-(4-((3-(trifluoromethyl)-4-(5-(trifluoromethyl)pyridin-2-yl)piperazin-1-yl)methyl)-1H-pyrazol-3-yl)-1H-pyrrole-3-carboxamide; (R)-5-(4-((3-methyl-4-(5-(trifluoromethyl)pyridin-2-yl)piperazin-1-yl)methyl)-1H-pyrazol-3-yl)furan-3-carboxylic acid; (R)-5-(4-((3-methyl-4-(5-(trifluoromethyl)pyridin-2-yl)piperazin-1-yl)methyl)-1H-pyrazol-3-yl)furan-3-carboxamide; (R)-3-hydroxy-1-(4-(4-((3-methyl-4-(5-(trifluoromethyl)pyridin-2-yl)piperazin-1-yl)methyl)-1H-pyrazol-3-yl)-5,6-dihydropyridin-1(2H)-yl)propan-1-one; (R)-6-(4-((3-methyl-4-(5-(trifluoromethyl)pyridin-2-yl)piperazin-1-yl)methyl)-1H-pyrazol-3-yl)-2-oxo-1,2-dihydropyridine-4-carbonitrile; (R)-2-methyl-4-((3-(5-phenyl-1H-imidazol-2-yl)-1H-pyrazol-4-yl)methyl)-1-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-1H-imidazole-4-carbonitrile; (R)-4-((3-methyl-4-(5-(trifluoromethyl)pyridin-2-yl)piperazin-1-yl)methyl)-N-(3-oxo-3,4-dihydro-2H-benzo[b][1,4]oxazin-7-yl)-1H-pyrazole-3-carboxamide; (R)-4-((3-methyl-4-(5-(trifluoromethyl)pyridin-2-yl)piperazin-1-yl)methyl)-N-(3-oxo-3,4-dihydro-2H-benzo[b][1,4]oxazin-6-yl)-1H-pyrazole-3-carboxamide; (R)—N-(4-(1H-imidazol-1-yl)phenyl)-4-((3-methyl-4-(5-(trifluoromethyl)pyridin-2-yl)piperazin-1-yl)methyl)-1H-pyrazole-3-carboxamide; (R)-4-((3-methyl-4-(5-(trifluoromethyl)pyridin-2-yl)piperazin-1-yl)methyl)-N-(4-(N-(5-methylisoxazol-3-yl)sulfamoyl)phenyl)-1H-pyrazole-3-carboxamide; (R)-3-(4-((3-methyl-4-(5-(trifluoromethyl)pyridin-2-yl)piperazin-1-yl)methyl)-1H-pyrazol-3-yl)-1H-indole; (R)-2-(4-((3-methyl-4-(5-(trifluoromethyl)pyridin-2-yl)piperazin-1-yl)methyl)-1H-pyrazol-3-yl)-1H-imidazole-4-carboxamide; (R)-4-(5-((3-methyl-4-(5-(trifluoromethyl)pyridin-2-yl)piperazin-1-yl)methyl)-2H-1,2,3-triazol-4-yl)benzonitrile and (R)-4-(5-((3-methyl-4-(4-(trifluoromethyl)phenyl)piperazin-1-yl)methyl)-2H-1,2,3-triazol-4-yl)benzonitrile.

The compounds of Formulas (I)-(XIX), pharmaceutically acceptable salts, solvates, N-oxides, prodrugs and isomers thereof, and pharmaceutical compositions provided herein also includes all suitable isotopic variations of such compounds, and pharmaceutically acceptable salts, solvates, N-oxides, prodrugs and isomers thereof, and pharmaceutical compositions. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that may be incorporated into the compounds of the invention and pharmaceutically acceptable salts thereof include but are not limited to isotopes of hydrogen, carbon, nitrogen and oxygen such as ²H, ³H, ¹¹C, ¹³C, ¹⁴C, ¹⁵N, ¹⁷O, ¹⁸O, ³⁵S, ¹⁸F, ³⁶Cl, and ¹²³I. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. In particular examples, ³H and ¹⁴C isotopes may be used for their ease of preparation and detectability. In other examples, substitution with isotopes such as ²H may afford certain therapeutic advantages resulting from greater metabolic stability, such as increased in vivo half-life or reduced dosage requirements. Isotopic variations of the compounds, and pharmaceutically acceptable salts, solvates, N-oxides, prodrugs and isomers thereof, and pharmaceutical compositions provided herein are prepared by conventional procedures using appropriate isotopic variations of suitable reagents.

### Processes for Making Compounds of Formula (I)

General procedures for preparing compounds of Formula (I) are described in the Examples, infra. In the reactions described, reactive functional groups, for example hydroxyl, amino, imino, thio or carboxy groups, where these are desired in the final product, may be protected to avoid their unwanted participation in the reactions. Conventional protecting groups may be used in accordance with standard practice (see e.g., T. W. Greene and P. G. M. Wuts in “Protective Groups in Organic Chemistry,” John Wiley and Sons, 1991).

In certain embodiments, the compounds of Formula (I) described herein are prepared as a pharmaceutically acceptable acid addition salt by reacting the free base form of the compound of Formula (I) with a pharmaceutically acceptable organic acid or inorganic acid. In other embodiments, a pharmaceutically acceptable base addition salt of compounds of Formula (I) described herein is prepared by reacting the free acid form of the compound of Formula (I) with a pharmaceutically acceptable organic base or inorganic base. Alternatively, the salt forms of the compounds of Formula (I) described herein are prepared using salts of the starting materials or intermediates. In certain embodiments, the compounds of Formula (I) described herein are in the form of other salts including, but not limited to, oxalates and trifluoroacetates. In certain embodiments, hemisalts of acids and bases are formed, for example, hemisulphate and hemicalcium salts.

Such pharmaceutically acceptable acid addition salts of compounds of Formula (I) include, but are not limited to, a hydrobromide, hydrochloride, sulfate, nitrate, succinate, maleate, formate, acetate, adipate, besylatye, bicarbonate/carbonate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate), hexanoate salt, bisulphate/sulphate, borate, camsylate, cyclamate, edisylate, esylate, gluceptate, gluconate, glucuronate, hexafluorophosphate, hibenzate, hydrochloride/chloride, hydrobromide/bromide, hydroiodide/iodide, isethionate, lactate, malate, malonate, mesylate, methylsulphate, naphthylate, 2-napsylate, nicotinate, orotate, oxalate, palmitate, pamoate, phosphate/hydrogen phosphate/dihydrogen phosphate, pyroglutamate, saccharate, stearate, tannate, tosylate, trifluoroacetate and xinofoate salts.

The organic acid or inorganic acids used to form certain pharmaceutically acceptable acid addition salts of compounds of Formula (I) include, but are not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid.

Such pharmaceutically acceptable base addition salt of a compound of Formula (I) include, but are not limited to, aluminium, arginine, benzathine, calcium, choline, diethylamine, diolamine, glycine, lysine, magnesium, meglumine, olamine, potassium, sodium, tromethamine and zinc salts.

In certain embodiments, the free acid or free base forms of the compounds of Formula (I) described herein are prepared from the corresponding base addition salt or acid addition salt from, respectively. For example a compound Formula (I) in an acid addition salt form is converted to the corresponding free base by treating with a suitable base (by way of example only, an ammonium hydroxide solution, a sodium hydroxide, and the like). For example, a compound of Formula (I) in a base addition salt form is converted to the corresponding free acid by treating with a suitable acid (by way of example only, hydrochloric acid).

In certain embodiments, the compounds of Formula (I) described herein in unoxidized form are prepared from N-oxides of compounds Formula (I) by treating with a reducing agent (by way of example only, sulfur, sulfur dioxide, triphenyl phosphine, lithium borohydride, sodium borohydride, phosphorus trichloride, tribromide, or the like) in a suitable inert organic solvent (by way of example only, acetonitrile, ethanol, aqueous dioxane, or the like) at 0 to 80° C.

In certain embodiments, prodrug derivatives of compounds Formula (I) described herein are prepared using methods known to those of ordinary skill in the art (e.g., for further details see Saulnier et al., (1994), Bioorganic and Medicinal Chemistry Letters, Vol. 4, p. 1985). For example, appropriate prodrugs are prepared by reacting a non-derivatized compound of Formula (I) with a suitable carbamylating agent (by way of example only, 1,1-acyloxyalkylcarbanochloridate, para-nitrophenyl carbonate, or the like).

In certain embodiments, the compounds of Formula (I) described herein are prepared as protected derivatives using methods known to those of ordinary skill in the art. A detailed description of the techniques applicable to the creation of protecting groups and their removal can be found in T. W. Greene, “Protecting Groups in Organic Chemistry,” 3^{rd} edition, John Wiley and Sons, Inc., 1999.

In certain embodiments, the compounds of Formula (I) described herein are prepared or formed, as solvates (e.g., hydrates). In certain embodiments, hydrates of compounds of Formula (I) are prepared by recrystallization from an aqueous/organic solvent mixture, using organic solvents such as dioxin, tetrahydrofuran or methanol.

In certain embodiments, the compounds of Formula (I) described herein are prepared as their individual stereoisomers. In other embodiments, the compounds of Formula (I) described herein are prepared as their individual stereoisomers by reacting a racemic mixture of the compound with an optically active resolving agent to form a pair of diastereoisomeric compounds, separating the diastereomers and recovering the optically pure enantiomers. In certain embodiments, resolution of enantiomers is carried out using covalent diastereomeric derivatives of the compounds of Formula (I), or by using dissociable complexes (e.g., crystalline diastereomeric salts). Diastereomers have distinct physical properties (e.g., melting points, boiling points, solubility, reactivity, etc.) and are readily separated by taking advantage of these dissimilarities. In certain embodiments, the diastereomers are separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions,” John Wiley And Sons, Inc., 1981.

Compounds of Formula (I) are made by processes described herein and as illustrated in the Examples. In certain embodiments, compounds of Formula (I) are made by:
(a) optionally converting a compound of the invention into a pharmaceutically acceptable salt;
(c) optionally converting a salt form of a compound of the invention to a non-salt form;
(d) optionally converting an unoxidized form of a compound of the invention into a pharmaceutically acceptable N-oxide;
(e) optionally converting an N-oxide form of a compound of the invention to its unoxidized form;
(f) optionally resolving an individual isomer of a compound of the invention from a mixture of isomers;
(g) optionally converting a non-derivatized compound of the invention into a pharmaceutically acceptable prodrug derivative; and
(h) optionally converting a prodrug derivative of a compound of the invention to its non-derivatized form.

Non-limiting examples of synthetic schemes used to make compounds of Formula (I) described herein are illustrated in reaction schemes (I)-(XI), wherein n, m, p, R¹, R², R³, R⁴, R¹¹ and R¹² are as defined herein.

Reaction scheme (I) illustrates the synthesis of substituted pyrazole having a structure of Formula (I) wherein L₁ is —(CR¹¹R¹²)ₚ—.

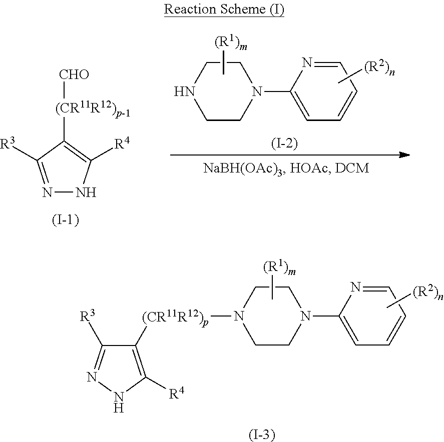

In Reaction Scheme (I) a compound of Formula (I) (I-3), wherein L₁ is —(CR¹¹R¹²)ₚ—, is prepared by reacting aldehyde (I-1) with amine (I-2) in the presence of a suitable solvent and an appropriate reducing agents. Solvents used in such reactions include, but are not limited to dichloromethane (DCM). Reducing agents used in such reactions include, but are not limited to, NaCNBH₃. Certain examples of aldehyde (I-1) are synthesized as described herein.

Reaction scheme (II) illustrates the synthesis of substituted pyrazole having a structure of Formula (I) wherein L₁ is —C(O)—.

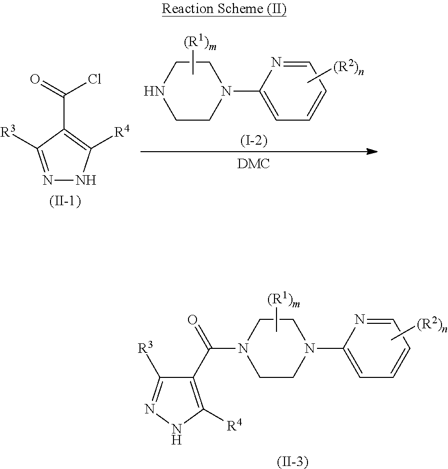

In Reaction Scheme (II) a compound of Formula (I) (II-3), wherein L₁ is —C(O)—, is prepared by reacting acid chloride (II-1) with amine (I-2) in the presence of a suitable solvent. Solvents used in such reactions include, but are not limited to dichloromethane (DCM).

Reaction scheme (III) illustrates the synthesis of substituted pyrazole having a structure of Formula (I) wherein L₁ is —SO₂—.

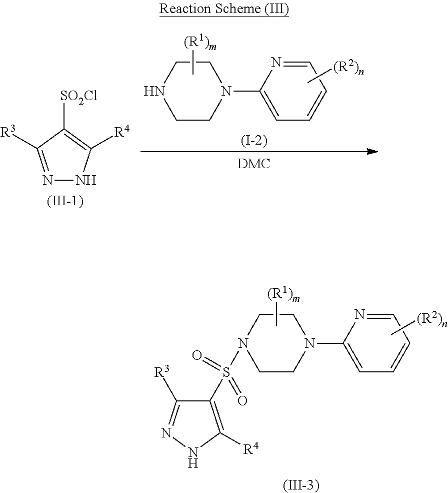

In Reaction Scheme (III) a compound of Formula (I) (III-3), wherein L₁ is —SO₂—, is prepared by reacting sulfonyl chloride (III-1) with amine (I-2) in the presence of a suitable solvent. Solvents used in such reactions include, but are not limited to dichloromethane (DCM).

Reaction scheme (IV) illustrates the synthesis of substituted pyrazole having a structure of Formula (I) wherein L₁ is —CH₂—.

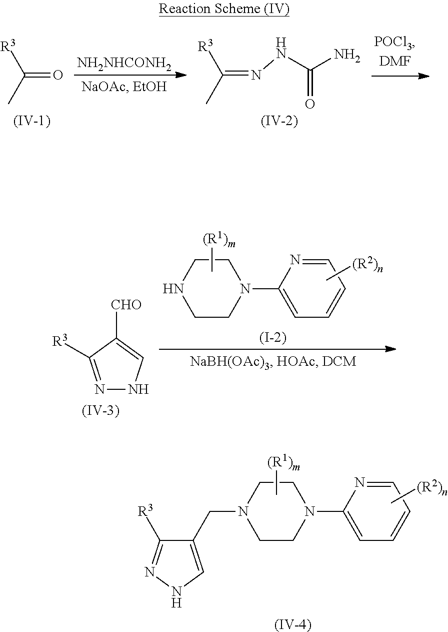

In Reaction Scheme (IV) compound of Formula (I) (IV-4), wherein L₁ is —CH₂—, is prepared by reacting aldehyde (IV-3) with amine (I-2) in the presence of a suitable solvent and an appropriate reducing agents. Aldehyde (IV-3) is prepared by reacting ketone (IV-1) with semicarbazide to form the semicarbazone (IV-2) which cyclizes in the presence of POCl₃ to give aldehyde (IV-3). Solvents used in such reactions include, but are not limited to dichloromethane (DCM). Reducing agents used in such reactions include, but are not limited to, NaCNBH₃.

Reaction scheme (V) illustrates another synthetic route to obtain substituted pyrazoles having a structure of Formula (I) wherein L₁ is —(CR¹¹R¹²)ₚ—.

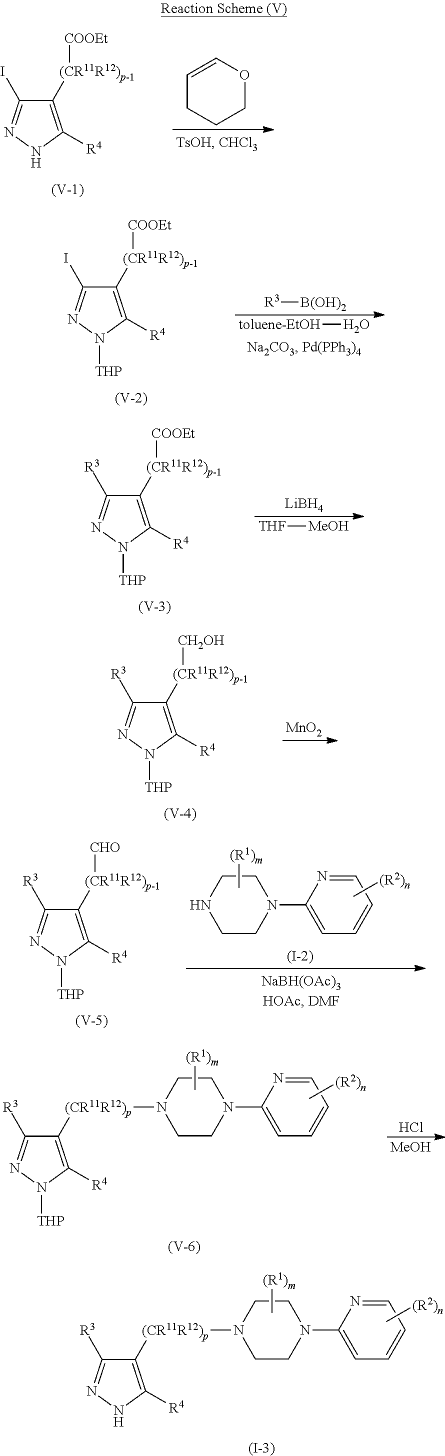

In Reaction Scheme (V) a protected pyrazole with an aldehyde substituent (V-5) is prepared by initially N protection of pyrazole (V-1), which is substituted with a halogen and an ester, to give the protected pyrazole (V-2). The halogen substituent of the protected pyrazole (V-2) is then reacted with a substituted boronic acid to give pyrazole (V-3). The ester group of pyrazole (V-3) is then reduced to give a pyrazole with an alcohol substituent (V-4), which is then oxidized to give the protected pyrazole with an aldehyde substituent (V-5). The aldehyde (V-5) is reacted with amine (I-2), in the presence of a suitable solvent and an appropriate reducing agent, to give the protected pyrazole (V-6), which is deprotected to give the pyrazole (I-3). Solvents used in such reactions include, but are not limited to dichloromethane (DCM). Reducing agents used in such reactions include, but are not limited to, NaCNBH₃.

Reaction scheme (VI) illustrates another synthetic route to obtain substituted pyrazoles having a structure of Formula (I) wherein L₁ is —(CR¹¹R¹²)ₚ—.

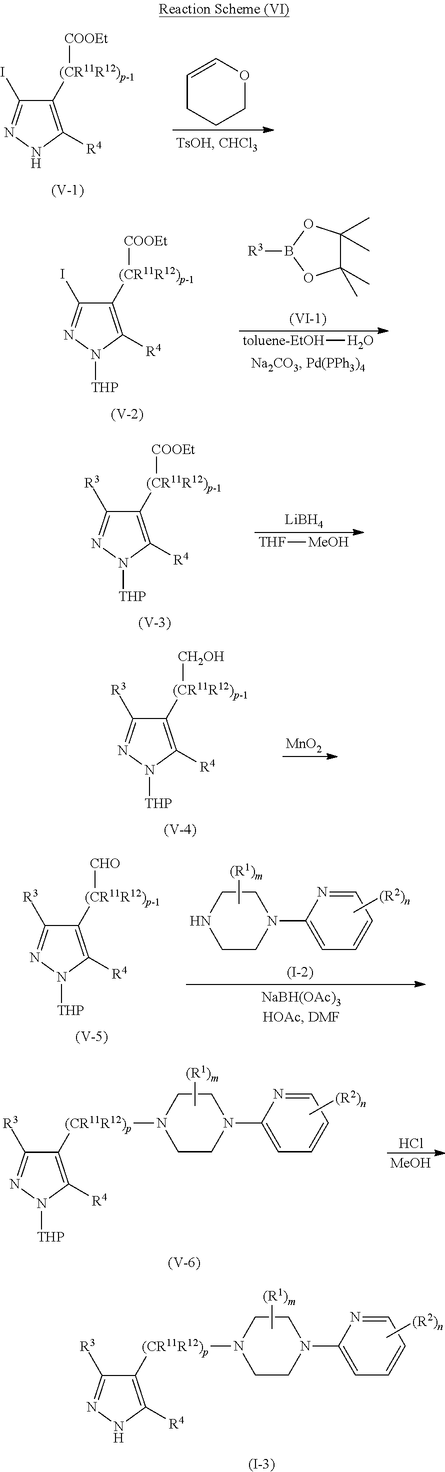

Reaction Scheme (VI) is similar to Reaction Scheme (V), however substituted boronate esters (VI-1) are used as boronic acid equivalents.

Reaction scheme (VII) illustrates the synthesis of substituted triazole having a structure of Formula (I) wherein L₁ is —(CR¹¹R¹²)ₚ—.

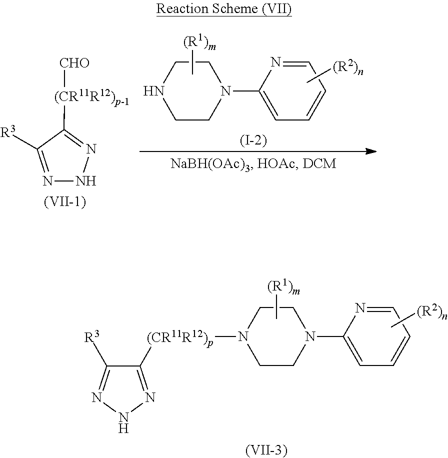

In Reaction Scheme (VII) a compound of Formula (I) (VII-3), wherein L₁ is —(CR¹¹R¹²)ₚ—, is prepared by reacting aldehyde (VII-1) with amine (I-2) in the presence of a suitable solvent and an appropriate reducing agents. Solvents used in such reactions include, but are not limited to dichloromethane (DCM). Reducing agents used in such reactions include, but are not limited to, NaCNBH₃. Certain examples of aldehyde (VII-1) are synthesized as described herein.

Reaction scheme (VIII) illustrates the synthesis of substituted pyrazole having a structure of Formula (I) wherein L₁ is —C(O)—.

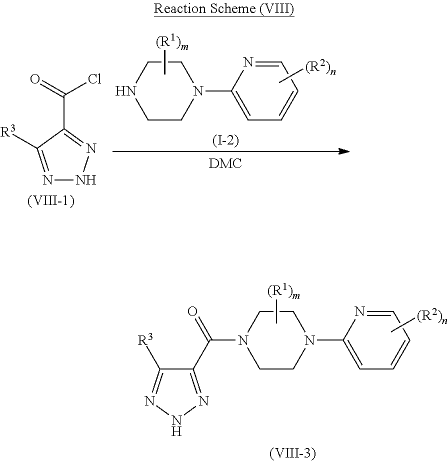

In Reaction Scheme (VIII) a compound of Formula (I) (VIII-3), wherein L₁ is —C(O)—, is prepared by reacting acid chloride (VIII-1) with amine (I-2) in the presence of a suitable solvent. Solvents used in such reactions include, but are not limited to dichloromethane (DCM).

Reaction scheme (IX) illustrates the synthesis of substituted triazole having a structure of Formula (I) wherein L₁ is —SO₂—.

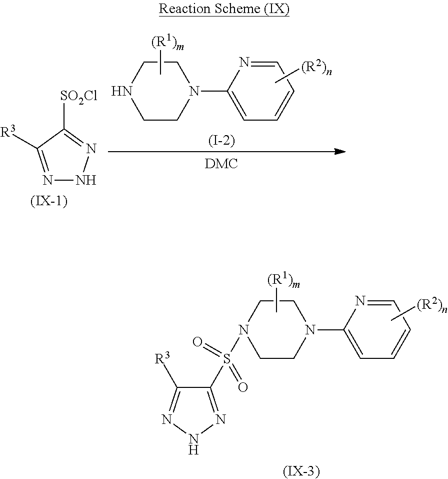

In Reaction Scheme (IX) a compound of Formula (I) (IX-3), wherein L₁ is —SO₂—, is prepared by reacting sulfonyl chloride (IX-1) with amine (I-2) in the presence of a suitable solvent. Solvents used in such reactions include, but are not limited to dichloromethane (DCM).

Reaction scheme (XI) illustrates the synthesis of substituted triazole having a structure of Formula (I) wherein L₁ is —CH₂—.

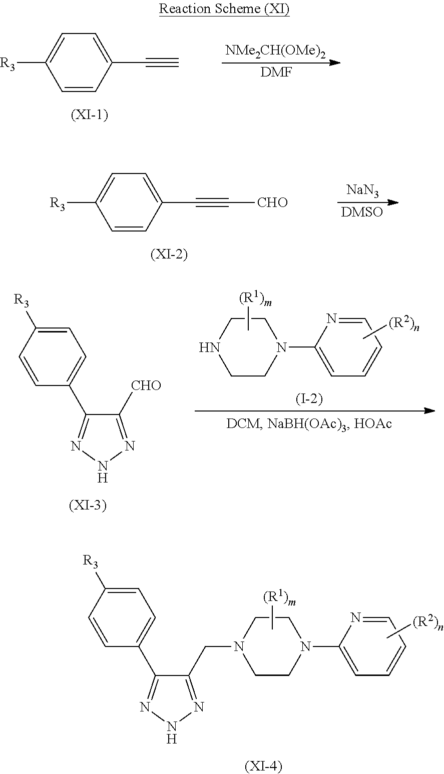

In Reaction Scheme (XI) compound of Formula (I) (XI-4), wherein L₁ is —CH₂—, is prepared by reacting the aldehyde substituted triazole (XI-3) with amine (I-2) in the presence of a suitable solvent and an appropriate reducing agents. Aldehyde substituted triazole (IV-3) is prepared by reacting nitrile (XI-1) with N,N-dimethylformamide dimethylacetal to form the aldehyde (XI-2) which cyclizes in the presence of NaN₃ to give aldehyde substituted triazole (XI-3). Solvents used in such reactions include, but are not limited to dichloromethane (DCM). Reducing agents used in such reactions include, but are not limited to, NaCNBH₃.

Detailed examples of the synthesis of a compound of Formula (I) can be found in the Examples, infra.

### Pharmacology and Utility

When a foreign antigen challenges the immune system it responds by launching a protective response that is characterized by the coordinated interaction of both the innate and acquired immune systems. These two interdependent systems fulfill two mutually exclusive requirements: speed (contributed by the innate system) and specificity (contributed by the adaptive system).

The innate immune system serves as the first line of defense against invading pathogens, holding the pathogen in check while the adaptive responses are matured. It is triggered within minutes of infection in an antigen-independent fashion, responding to broadly conserved patterns in the pathogens (though it is not non-specific, and can distinguish between self and pathogens). Crucially, it also generates the inflammatory and co-stimulatory milieu (sometimes referred to as the danger signal) that potentiates the adaptive immune system and steers (or polarizes it) towards the cellular or humoral responses most appropriate for combating the infectious agent.

The adaptive response becomes effective over days or weeks, but ultimately provides the fine antigenic specificity required for complete elimination of the pathogen and the generation of immunologic memory. It is mediated principally by T and B cells that have undergone germline gene rearrangement and are characterized by specificity and long-lasting memory. However, it also involves the recruitment of elements of the innate immune system, including professional phagocytes (macrophages, neutrophils etc.) and granulocytes (basophils, eosinophils etc.) that engulf bacteria and even relatively large protozoal parasites. Once an adaptive immune response has matured, subsequent exposure to the pathogen results in its rapid elimination due to highly specific memory cells have been generated that are rapidly activated upon subsequent exposure to their cognate antigen.

Autoimmune diseases, are defined by (i) humoral or autoantibody response to a self antigen (by way of example only, Graves' primary hyperthyroidism with antibodies to the TSH receptor), or (ii) cellular response wherein immune cells destroy nonimmune cells from which the self-antigen is derived (by way of example only, the thyrocyte (Hashimoto's thyroiditis) or pancreatic β-islet cell (Type 1 diabetes). Many autoimmune diseases are a combination of both phenomena, for instance, Hashimoto's and Type 1 diabetes also have auto-antibodies, anti-thyroid peroxidase (TPO) or anti-glutamic acid decarboxylase (GAD)/Islet Cell. Autoimmune diseases often have an inflammatory component including, but not limited to, increases in adhesion molecules (by way of example only, vascular cell adhesion molecule-1 (VCAM-1), and altered leukocyte adhesion to the vasculature such as, by way of example only, colitis, systemic lupus, systemic sclerosis, and the vascular complications of diabetes.

Inositol 1,4,5-trisphosphate 3-kinase B (ITPKB) is a protein encoded by the human gene itpkb and the activity of this encoded protein is responsible for regulating the levels of a large number of inositol polyphosphates that are important in cellular signaling. Unlike protein kinases, ITPKB does not phosphorylate other proteins, rather ITPKB regulates inositol phosphate metabolism by phosphorylation of second messenger inositol 1,4,5-trisphosphate (IP₃) to inositol 1,3,4,5-tetrakisphosphate (IP₄). ITPKB activity is controlled by both calcium/calmodulin and protein phosphorylation mechanisms.

Inositol 1,4,5-trisphosphate (IP₃), together with diacylglycerol, is a secondary messenger molecule used in signal transduction in biological cells. The main functions of IP₃ are to mobilize Ca²⁺ from storage organelles and to regulate cell proliferation and other cellular reactions. IP₃ binds to and activates the InsP₃ receptor on the membrane of the sarcoplasmic reticulum (SR) opens a calcium channel, resulting in the release of Ca²⁺ into the sarcoplasm. This increase in Ca²⁺ activates the ryanodine receptor-operated channel on the SR, leading to a further increase in the Ca²⁺. Inositol 1,4,5-trisphosphate (IP₃) is a critical mediator of T cell receptor (TCR) induced Ca²⁺ release from internal stores. By modulating the levels of IP3, 1,3,4,5-tetrakisphosphate (IP₄) plays a role in calcium signaling in nonlymphoid cells. Inositol 1,4,5-trisphosphate (IP₃) is made by hydrolysis of phosphatidylinositol 4,5-bisphosphate (PIP₂) by phospholipase C. PIP₂ is a phospholipid that is located in the plasma membrane.

ITPKB is one of three inositol trisphosphate kinases (ITPKA, ITPKB and ITPKC) that convert IP₃ to IP₄. Targeted knockout of each of the genes expressing these proteins in the mouse revealed that ITPKB alone is uniquely required for lymphocyte development and activation. This is likely due to the unique properties of ITPKB and its restricted expression pattern. ITPKA is expressed solely in the brain, and the knockout mice show significantly enhanced long-term potentiation (LTP) in the hippocampal CA1 region without demonstrating enhancement or other abnormality of learning and memory. ITPKC is expressed ubiquitously and likely serves as a housekeeping function for regulating IP4 levels as indicated by the normal phenotype of mice lacking ITPKC. ITPKB/C double-knockout mice are viable and have blocks in T cell development that are identical to the ITPKB−/− mice. Importantly, mice lacking ITPKB are fertile and show no obvious defects in a variety of metabolic or neurological parameters.

ITPKB−/− mice lack mature T cells, therefore the function of ITPKB in mature T cells is unclear. However, in contrast to T cells, ITPKB−/− mice have mature B cells, but their numbers are reduced by about 70%. Analysis of this phenotype shows defects in B cell receptor (BCR) driven B cell development and activation. In particular, ITPKB−/− mice contain large numbers of B cells that resemble tolerant B cells and have defective antibody responses to a T cell independent antigen. In addition, ITPKB−/− B cells displayed enhanced store operated calcium (SOC) channel activity following BCR stimulation. Cell permeable IP₄ can block SOC channel activity in normal B cells and addition of IP₄ reverses elevated SOC activity in ITPKB−/− B cells. Thus IP₄ regulates BCR signaling by acting to limit BCR driven Ca²⁺ influx. Sustained BCR stimulation prevents B cell differentiation into antibody secreting cells. Thus, inhibitors of ITPKB can block (auto)antibody production by dysregulating BCR driven Ca²⁺ influx.

The diseases and conditions that are associated with or mediated by abnormal B cell proliferation, include, but are not limited to, B cell lymphoma, chronic transplant rejection, immune-mediated disease, autoimmune mediated diseases, and anaphylaxis and many complement mediated diseases. Such immune mediated disorders include, but are not limited to, allergy and psoriasis. Such autoimmune mediated disorders include, but are not limited to, rheumatoid arthritis (RA), systematic lupus erythematosus (SLE), hemolytic anemia, lupus, primary binary cirrhosis (PBC) and idiopathic thrombocytopenic purpura (ITP). Such allergy disorders include, but are not limited to, respiratory diseases and dermatolgical disorders. Respiratory diseases include but are not limited to, asthma, rhinitis, COPD, asthma, bronchial asthma, allergic asthma, intrinsic asthma, extrinsic asthma, exercise-induced asthma, drug-induced asthma (including aspirin and NSAID-induced) and dust-induced asthma, chronic obstructive pulmonary disease (COPD); bronchitis, acute and chronic rhinitis including rhinitis medicamentosa, and vasomotor rhinitis; perennial and seasonal allergic rhinitis including rhinitis nervosa (hay fever). Dermatological diseases and/or disorders include, but are not limited to, dermatitis and eczema such as, by way of example only, atopic dermatitis, seborrhoeic dermatitis (Dandruff, Cradle cap), diaper rash, urushiol-induced contact dermatitis, contact dermatitis, erythroderma, lichen simplex chronicus, prurigo nodularis, itch, pruritus ani, nummular dermatitis, dyshidrosis and pityriasis alba.

### Routes of Administration and Pharmaceutical Compositions

For the therapeutic uses of compounds of Formula (I), or pharmaceutically acceptable salts, solvates, N-oxides, prodrugs and isomers thereof, described herein, such compounds are administered in therapeutically effective amounts either alone or as part of a pharmaceutical composition. Accordingly, provided herein are pharmaceutical compositions, which comprise at least one compound of Formulas (I) described herein, pharmaceutically acceptable salts and/or solvates thereof, and one or more pharmaceutically acceptable carriers, diluents, or excipients. In addition, such compounds and compositions are administered singly or in combination with one or more additional therapeutic agents. The routes of administration of compounds of Formula (I) and pharmaceutical compositions include, but are not limited to, oral administration, intravitreal administration, rectal administration, parenteral, intravenous administration, intraperitoneal administration, intramuscular administration, inhalation, transmucosal administration, pulmonary administration, intestinal administration, subcutaneous administration, intramedullary administration, intrathecal administration, direct intraventricular, intranasal administration, topical administration, ophthalmic administration or otic administration.

In certain embodiments, compounds of Formula (I) or pharmaceutical compositions described herein are administered locally, while in other embodiments compounds of Formula (I) or pharmaceutical composite described herein are administered systemically. Local administration includes, but is not limited to, injection into an organ, optionally in a depot or sustained release formulation. Systemic administration includes, but is not limited to, oral administration or intravenous administration. In other embodiments, compounds of Formula (I) or pharmaceutical compositions described herein are administered in a targeted drug delivery system, such as, by way of example only, in a liposome coated with organ-specific antibody. The liposome is targeted to and taken up selectively by the organ. In other embodiments, compounds of Formula (I) or pharmaceutical compositions described herein are administered in the form of a rapid release formulation, while in other embodiments, compounds of Formula (I) or pharmaceutical compositions described herein are administered in the form of an extended release formulation . In other embodiments, compounds of Formula (I) or pharmaceutical compositions described herein are administered in the form of an intermediate release formulation.

The therapeutically effective amount will vary depending on, among others, the disease indicated, the severity of the disease, the age and relative health of the subject, the potency of the compound administered, the route of administration and the treatment desired. In certain embodiments, satisfactory results are indicated to be obtained at daily dosages daily dosage of a compound of Formula (I) from about 0.03 to 2.5 mg/kg per body weight. In certain embodiments, the daily dosage of a compound of Formula (I), administered orally, is in the range from 0.05 micrograms per kilogram body weight (μg/kg) to 100 micrograms per kilogram body weight (μg/kg). In certain embodiments, the daily dosage of a compound of Formula (I), administered topically, is in the range from 0.05 micrograms per kilogram body weight (ug/kg) to 100 micrograms per kilogram body weight (μg/kg). In other embodiments, the daily dosage of a compound of Formula (I), administered parenterally, is in the range from 0.05 micrograms per kilogram body weight (μg/kg) to 100 milligrams per kilogram body weight (mg/kg). In certain embodiments, the daily dosage of a compound of Formula (I), administered intrermuscularlly, is in the range from 0.05 micrograms per kilogram body weight (μg/kg) to 100 micrograms per kilogram body weight (μg/kg). An indicated daily dosage in the larger mammal, e.g. humans, is in the range from about 0.5 mg to about 100 mg of a compound of Formula (I), conveniently administered, e.g. in divided doses up to four times a day or in controlled release form. In certain embodiment, unit dosage forms for oral administration comprise from about 1 to 50 mg of a compound of Formula (I).

Other aspects provided herein are processes for the preparation of pharmaceutical composition which comprise at least one compound of Formula (I) described herein. In certain embodiments, such processes include admixing a compound of Formula (I) described herein with one or more pharmaceutically acceptable carriers, diluents or excipients. In certain embodiments, the pharmaceutical compositions comprise a compound of Formula (I) in free form or in a pharmaceutically acceptable salt or solvate form. In certain embodiments, the pharmaceutical compositions comprising a compound of Formula (I) in free form or in a pharmaceutically acceptable salt or solvate form, in association with at least one pharmaceutically acceptable carrier, diluent or excipient are manufactured by mixing, dissolving, granulating dragee-making, levigating, emulsifying, encapsulating, entrapping or compression processes and/or coating methods. In other embodiments, such compositions are optionally contain excipients, such as preserving, stabilizing, wetting or emulsifying agents, solution promoters, salts for regulating the osmotic pressure and/or buffers. In other embodiments, such compositions are sterilized.

### Oral Dosage Forms

In certain embodiments, the pharmaceutical compositions containing at least one compound of Formula (I) are administered orally as discrete dosage forms, wherein such dosage forms include, but are not limited to, capsules, gelatin capsules, caplets, tablets, chewable tablets, powders, pills, dragees, granules, liquids, gels, syrups, flavored syrups, elixirs, slurries, solutions or suspensions in aqueous or non-aqueous liquids, edible foams or whips, and oil-in-water liquid emulsions or water-in-oil liquid emulsions. The capsules, gelatin capsules, caplets, tablets, chewable tablets, powders or granules, used for the oral administration of at least one compound of Formula (I) are prepared by admixing at least one compound of Formula (I) (active ingredient) together with at least one excipient using conventional pharmaceutical compounding techniques. Non-limiting examples of excipients used in oral dosage forms described herein include, but are not limited to, binders, fillers, disintegrants, lubricants, absorbents, colorants, flavors, preservatives and sweeteners.

Non-limiting examples of such binders include, but are not limited to, corn starch, potato starch, starch paste, pre-gelatinized starch, or other starches, sugars, gelatin, natural and synthetic gums such as acacia, sodium alginate, alginic acid, other alginates, tragacanth, guar gum, cellulose and its derivatives (by way of example only, ethyl cellulose, cellulose acetate, carboxymethyl cellulose calcium, sodium carboxymethylcellulose, methyl cellulose, hydroxypropyl methylcellulose and microcrystalline cellulose), magnesium aluminum silicate, polyvinyl pyrrolidone and combinations thereof.

Non-limiting examples of such fillers include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein are present in from about 50 to about 99 weight percent of the pharmaceutical composition or dosage form.

Non-limiting examples of such disintegrants include, but are not limited to, agar-agar, alginic acid, sodium alginate, calcium carbonate, sodium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and combinations thereof. In certain embodiments, the amount of disintegrant used in the pharmaceutical compositions provided herein is from about 0.5 to about 15 weight percent of disintegrant, while in other embodiments the amount is from about 1 to about 5 weight percent of disintegrant.

Non-limiting examples of such lubricants include, but are not limited to, sodium stearate, calcium stearate, magnesium stearate, stearic acid, mineral oil, light mineral oil, glycerin, sorbitol, mannitol, polyethylene glycol, other glycols, sodium lauryl sulfate, talc, hydrogenated vegetable oil (by way of example only, peanut oil, cottonseed oil, sunflower oil, sesame oil, olive oil, corn oil, and soybean oil), zinc stearate, sodium oleate, ethyl oleate, ethyl laureate, agar, silica, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.) and combinations thereof. In certain embodiments, the amount of lubricants used in the pharmaceutical compositions provided herein is in an amount of less than about 1 weight percent of the pharmaceutical compositions or dosage forms.

Non-limiting examples of such diluents include, but are not limited to, lactose, dextrose, sucrose, mannitol, sorbitol, cellulose, glycine or combinations thereof.

In certain embodiments, tablets and capsules are prepared by uniformly admixing at least one compound of Formula (I) (active ingredients) with liquid carriers, finely divided solid carriers, or both, and then shaping the product into the desired presentation if necessary. In certain embodiments, tablets are prepared by compression. In other embodiments, tablets are prepared by molding.

In certain embodiments, at least one compound of Formula (I) is orally administered as a controlled release dosage form. Such dosage forms are used to provide slow or controlled-release of one or more compounds of Formula (I). Controlled release is obtained using, for example, hydroxypropylmethyl cellulose, other polymer matrices, gels, permeable membranes, osmotic systems, multilayer coatings, microparticles, liposomes, microspheres, or a combination thereof. In certain embodiments, controlled-release dosage forms are used to extend activity of the compound of Formula (I), reduce dosage frequency, and increase patient compliance.

Administration of compound of Formula (I) as oral fluids such as solution, syrups and elixirs are prepared in unit dosage forms such that a given quantity of solution, syrups or elixirs contains a predetermined amount of a compound of Formula (I). Syrups are prepared by dissolving the compound in a suitably flavored aqueous solution, while elixirs are prepared through the use of a non-toxic alcoholic vehicle. Suspensions are formulated by dispersing the compound in a non-toxic vehicle. Non-limiting examples of excipients used in as oral fluids for oral administration include, but are not limited to, solubilizers, emulsifiers, flavoring agents, preservatives, and coloring agents. Non-limiting examples of solubilizers and emulsifiers include, but are not limited to, water, glycols, oils, alcohols, ethoxylated isostearyl alcohols and polyoxy ethylene sorbitol ethers. Non-limiting examples of preservatives include, but are not limited to, sodium benzoate. Non-limiting examples of flavoring agents include, but are not limited to, peppermint oil or natural sweeteners or saccharin or other artificial sweeteners.

### Parenteral Dosage Forms

In certain embodiments pharmaceutical compositions containing at least one compound of Formula (I) are administered parenterally by various routes including, but not limited to, subcutaneous, intravenous (including bolus injection), intramuscular, and intraarterial.

Such parenteral dosage forms are administered in the form of sterile or sterilizable injectable solutions, suspensions, dry and/or lyophylized products ready to be dissolved or suspended in a pharmaceutically acceptable vehicle for injection (reconstitutable powders) and emulsions. Vehicles used in such dosage forms include, but are not limited to, Water for Injection USP; aqueous vehicles such as, but not limited to, Sodium Chloride Injection, physiological saline buffer, Ringer's Injection solution, Dextrose Injection, Dextrose and Sodium Chloride Injection, and Lactated Ringer's Injection solution; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In certain embodiments, a compound of Formula (I) or composition containing one or more compounds of Formula (I) is parenteral administration by bolus injection. In other embodiments, a compound of Formula (I) or composition containing one or more compounds of Formula (I) is parenteral administration by continuous infusion. Formulations for injection are presented in unit dosage form, by way of example only, in ampoules or formulations for injection are presented in multi-dose containers, with an added preservative. The compositions may take such forms as suspensions, solutions or emulsions in oily or aqueous vehicles, and may contain formulatory agents such as suspending, stabilizing and/or dispersing agents.

### Transdermal Administration

In certain embodiments pharmaceutical compositions containing at least one compound of Formula (I) are administered transdermally. Such transdermal dosage forms include “reservoir type” or “matrix type” patches, which are applied to the skin and worn for a specific period of time to permit the penetration of a desired amount of a compound of Formula (I). By way of example only, such transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. In other embodiments, matrix transdermal formulations are used. In certain embodiments transdermal administration is used to provide continuous, while in other embodiments transdermal administration is used to provide discontinuous infusion of a compound of Formula (I) in controlled amounts.

In certain embodiments, the rate of absorption is slowed by using rate-controlling membranes or by trapping the compound within a polymer matrix or gel. In certain embodiments, transdermal delivery is via a transdermal patch.

Formulations for transdermal delivery of a compound of Formula (I) include an effective amount of a compound of Formula (I), a carrier and an optional diluent. A carrier includes, but is not limited to, absorbable pharmacologically acceptable solvents to assist passage through the skin of the host, such as water, acetone, ethanol, ethylene glycol, propylene glycol, butane-1,3-diol, isopropyl myristate, isopropyl palmitate, mineral oil, and combinations thereof.

In certain embodiments, such transdermal delivery systems include penetration enhancers to assist in delivering one or more compound of Formula (I) to the tissue. Such penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

In other embodiments, the pH of such a transdermal pharmaceutical composition or dosage form, or of the tissue to which the pharmaceutical composition or dosage form is applied, is adjusted to improve delivery of one or more compounds of Formula (I). In other embodiments, the polarity of a solvent carrier, its ionic strength, or tonicity are adjusted to improve delivery. In other embodiments, compounds such as stearates are added to advantageously alter the hydrophilicity or lipophilicity of one or more compound of Formula (I) so as to improve delivery. In certain embodiments, such stearates serve as a lipid vehicle for the formulation, as an emulsifying agent or surfactant, and as a delivery-enhancing or penetration-enhancing agent. In other embodiments, different salts, hydrates or solvates of the compound of Formula (I) are used to further adjust the properties of the resulting composition.

In other embodiments, transdermal delivery of the compound of Formula (I) is accomplished by means of iontophoretic patches and the like

### Topical Dosage Forms

In certain embodiments at least one compound of Formula (I) is administered by topical application of pharmaceutical composition containing at least one compound of Formula (I) in the form of lotions, gels, ointments solutions, emulsions, suspensions or creams. Suitable formulations for topical application to the skin are aqueous solutions, ointments, creams or gels, while formulations for ophthalmic administration are aqueous solutions. Such formulations optionally contain solubilizers, stabilizers, tonicity enhancing agents, buffers and preservatives.

Such topical formulations include at least one carrier, and optionally at least one diluent. Such carriers and diluents include, but are not limited to, water, acetone, ethanol, ethylene glycol, propylene glycol, butane-1,3-diol, isopropyl myristate, isopropyl palmitate, mineral oil, and combinations thereof.

In certain embodiments, such topical formulations include penetration enhancers to assist in delivering one or more compound of Formula (I) to the tissue. Such penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

### Pulmonary Administration

In certain embodiments pharmaceutical compositions containing at least one compound of Formula (I) are administered by inhalation. Dosage forms for inhaled administration are formulated as aerosols or dry powders. Aerosol formulations for inhalation administration comprise a solution or fine suspension of at least one compound of Formula (I) in a pharmaceutically acceptable aqueous or non-aqueous solvent. In addition, such pharmaceutical compositions optionally comprise a powder base such as lactose, glucose, trehalose, mannitol or starch, and optionally a performance modifier such as L-leucine or another amino acid, and/or metals salts of stearic acid such as magnesium or calcium stearate.

In certain embodiments, compound of Formula (I) are be administered directly to the lung by inhalation using a Metered Dose Inhaler (“MDI”), which utilizes canisters that contain a suitable low boiling propellant, e.g., dichlorodifluoromethane, trichlorofluoromethane, dichlorotetrafluoroethane, carbon dioxide or other suitable gas, or a Dry Powder Inhaler (DPI) device which uses a burst of gas to create a cloud of dry powder inside a container, which is then be inhaled by the patient. In certain embodiments, capsules and cartridges of gelatin for use in an inhaler or insufflator are formulated containing a powder mixture of a compound of Formula (I) and a powder base such as lactose or starch. In certain embodiments, compound of Formula (I) are delivered to the lung using a liquid spray device, wherein such devices use extremely small nozzle holes to aerosolize liquid drug formulations that can then be directly inhaled into the lung. In other embodiments, compound of Formula (I) are delivered to the lung using a nebulizer device, wherein a nebulizers creates an aerosols of liquid drug formulations by using ultrasonic energy to form fine particles that can be readily inhaled. In other embodiments, compound of Formula (I) are delivered to the lung using an electrohydrodynamic (“EHD”) aerosol device wherein such EHD aerosol devices use electrical energy to aerosolize liquid drug solutions or suspensions.

In certain embodiments, the pharmaceutical composition containing at least one compound of Formula (I), or pharmaceutically acceptable salts and solvates thereof, described herein, also contain one or more absorption enhancers. In certain embodiments, such absorption enhancers include, but are not limited to, sodium glycocholate, sodium caprate, N-lauryl-β-D-maltopyranoside, EDTA, and mixed micelles.

In certain embodiments pharmaceutical compositions containing at least one compound of Formula (I) are administered nasally. The dosage forms for nasal administration are formulated as aerosols, solutions, drops, gels or dry powders.

### Rectal Administration

In certain embodiments pharmaceutical compositions containing at least one compound of Formula (I) are administered rectally in the form of suppositories, enemas, retention enemas ointment, creams rectal foams or rectal gels. In certain embodiments such suppositories are prepared from fatty emulsions or suspensions, cocoa butter or other glycerides.

### Depot Administration

In certain embodiments pharmaceutical compositions containing at least one compound of Formula (I) are formulated as a depot preparation. Such long acting formulations are administered by implantation (for example subcutaneously or intramuscularly) or by intramuscular injection. In certain embodiments, such formulations include polymeric or hydrophobic materials (for example, as an emulsion in an acceptable oil) or ion exchange resins, or as sparingly soluble derivatives, for example, as a sparingly soluble salt.

In certain embodiments injectable depot forms are made by forming microencapsulated matrices of the compound of Formula (I) in biodegradable polymers. The rate of compound of Formula (I) release is controlled by varying the ratio of compound of Formula (I) to polymer and the nature of the particular polymer employed. In other embodiments, depot injectable formulations are prepared by entrapping the compound of Formula (I) in liposomes or microemulsions.

Ophthalmic Administration

In certain embodiments, a compound of Formula (I) or pharmaceutical composition described herein are ophthalmically administered to the eye. Administration to the eye generally results in direct contact of the agents with the cornea, through which at least a portion of the administered agents pass. In certain embodiments, such compounds of Formula (I) or pharmaceutical compositions have an effective residence time in the eye of about 2 to about 24 hours. In certain embodiments, such compounds of Formula (I) or pharmaceutical compositions have an effective residence time in the eye of about 4 to about 24 hours. In certain embodiments, such compounds of Formula (I) or pharmaceutical compositions have an effective residence time in the eye of about 6 to about 24 hours.

Ophthalmic administration, as used herein, includes, but is not limited to, topical administration, intraocular injection, subretinal injection, intravitreal injection, periocular administration, subconjuctival injections, retrobulbar injections, intracameral injections (including into the anterior or vitreous chamber), sub-Tenon's injections or implants, ophthalmic solutions, ophthalmic suspensions, ophthalmic ointments, ocular implants and ocular inserts, intraocular solutions, use of iontophoresis, incorporation in surgical irrigating solutions, and packs (by way of example only, a saturated cotton pledget inserted in the fornix). In certain embodiments, the compounds of Formula (I) or pharmaceutical composition described herein are formulated as an ophthalmic composition and are administered topically to the eye. Such topically administered ophthalmic compositions include, but are not limited to, solutions, suspensions, gels or ointments.

In certain embodiments the pharmaceutical compositions, comprising at least one compound of Formula (I) described herein, used for ophthalmic administration take the form of a liquid where the compositions are present in solution, in suspension or both. In some embodiments, a liquid composition includes a gel formulation. In other embodiments, the liquid composition is aqueous. In other embodiments, such liquid compositions take the form of an ointment. In certain embodiments pharmaceutical compositions containing at least one compound of Formula (I) are administered ophthamically as eye drops formulated as aqueous solutions that optionally contain solubilizers, stabilizers, tonicity enhancing agents, buffers and preservatives. A desired dosage is administered via a known number of drops into the eye. By way of example only, for a drop volume of 25 μl, administration of 1-6 drops delivers 25-150 μl of the composition. In certain embodiments, the aqueous compositions contain from about 0.01% to about 50% weight/volume of a compound of Formula (I). In other embodiments, the aqueous compositions contain from about 0.1% to about 20% weight/volume of a compound of Formula (I). In still other embodiments, the aqueous compositions contain from about 0.2% to about 10% weight/volume of a compound of Formula (I). In certain embodiments, the aqueous compositions contain from about 0.5% to about 5%, weight/volume of a compound of Formula (I).

In certain embodiments the aqueous compositions have an ophthalmically acceptable pH and osmolality. In certain embodiments the aqueous compositions include one or more ophthalmically acceptable pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

In certain embodiments the compositions also include also include one or more ophthalmically acceptable salts in an amount required to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In certain embodiments the aqueous compositions also contain one or more polymers as suspending agents. Such polymers include, but are not limited to, water-soluble polymers such as cellulosic polymers described herein, (for example only, hydroxypropyl methylcellulose), and water-insoluble polymers described herein (for example only, cross-linked carboxyl-containing polymers). In certain embodiments, the aqueous compositions also include an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In certain embodiments the compositions also include ophthalmically acceptable solubilizing agents to aid in the solubility of a compound of Formula (I). The term “solubilizing agent” generally includes agents that result in formation of a micellar solution or a true solution of the agent. In certain embodiments, ophthalmically acceptable nonionic surfactants including, but not limited to, polysorbate 80 are used as solubilizing agents. In other embodiments, ophthalmically acceptable glycols including, but not limited to, polyglycols, polyethylene glycol 400, and glycol ethers are used as solubilizing agents.

In certain embodiments the compositions also include one or more ophthalmically acceptable surfactants to enhance physical stability or for other purposes. Such nonionic surfactants include, but are not limited to, polyoxyethylene fatty acid glycerides and vegetable oils (by way of example only, polyoxyethylene (60) hydrogenated castor oil) and polyoxyethylene alkylethers and alkylphenyl ethers (by way of example only, octoxynol 10 and octoxynol 40).

In certain embodiments the compositions also include one or more ophthalmically acceptable preservatives to inhibit microbial activity. Such preservatives include, but are not limited to mercury-containing substances such as merfen and thiomersal; stabilized chlorine dioxide; and quaternary ammonium compounds such as benzalkonium chloride, cetyltrimethylammonium bromide and cetylpyridinium chloride.

In certain embodiments the compositions also include one or more antioxidants to enhance chemical stability where required. Such antioxidants include, but are not limited to, ascorbic acid and sodium metabisulfite.

In certain embodiments, the aqueous compositions provided herein are packaged in single-dose non-reclosable containers, while in other embodiments the aqueous compositions provided herein are packaged in multiple-dose reclosable containers wherein a preservative is included in the composition.

### Otic Administration

In certain embodiments pharmaceutical compositions containing at least one compound of Formula (I) are administered otically as ear drops. Such formulations are aqueous solutions that optionally contain solubilizers, stabilizers, tonicity enhancing agents, buffers and preservatives.

### Combination Therapies

In certain embodiments, a compound of Formulas (I)-(XIX) described herein, or a pharmaceutically acceptable salt or solvate thereof, or a pharmaceutical composition containing at least one compound of Formula (I)-(XIX) described herein, is administered alone (without an additional therapeutic agent) for the treatment of one or more of the disease and/or disorders associated with ITBPK activity described herein.

In other embodiments, a compound of Formulas (I)-(XIX) described herein, or a pharmaceutically acceptable salt or solvate thereof, or a pharmaceutical composition containing at least one compound of Formula (I)-(XIX) described herein, is administered in combination with one or more additional therapeutic agents, for the treatment of one or more of the disease and/or disorders associated with ITPBK activity described herein.

In other embodiments, a compound of Formulas (I)-(XIX) described herein, or a pharmaceutically acceptable salt or solvate thereof, or a pharmaceutical composition containing at least one compound of Formula (I)-(XIX) described herein, is formulated in combination with one or more additional therapeutic agents and administered for the treatment of one or more of the disease and/or disorders associated with ITPKB activity described herein.

In a compound of Formulas (I)-(XIX) described herein, or a pharmaceutically acceptable salt or solvate thereof, or a pharmaceutical composition containing at least one compound of Formula (I)-(XIX) described herein, is administered sequentially with one or more additional therapeutic agents, for the treatment of one or more of the disease and/or disorders associated with ITPKB activity described herein.

In other embodiments, the combination treatments provided herein include administration of a compound of Formula (I)-(XIX) described herein, or a pharmaceutically acceptable salt or solvate thereof, or a pharmaceutical composition containing a compound of Formula (I)-(XIX), prior to administration of one or more additional therapeutic agents, for the treatment of one or more of the disease and/or disorders associated with ITPKB activity described herein.

In other embodiments, the combination treatments provided herein include administration of a compound of Formula (I)-(XIX) described herein, or a pharmaceutically acceptable salt or solvate thereof, or a pharmaceutical composition containing a compound of Formula (I)-(XIX), subsequent to administration of one or more additional therapeutic agents, for the treatment of one or more of the disease and/or disorders associated with ITPKB activity described herein.

In certain embodiments, the combination treatments provided herein include administration of a compound of Formula (I)-(XIX) described herein, or a pharmaceutically acceptable salt or solvate thereof, or a pharmaceutical composition containing a compound of Formula (I)-(XIX), concurrently with one or more additional therapeutic agents, for the treatment of one or more of the disease and/or disorders associated with ITPKB activity described herein.

In certain embodiments, the combination treatments provided herein include administration of a compound of Formula (I)-(XIX) described herein, or a pharmaceutically acceptable salt or solvate thereof, or a pharmaceutical composition containing a compound of Formula (I)-(XIX) formulated with one or more additional therapeutic agents, for the treatment of one or more of the disease and/or disorders associated with ITPKB activity described herein.

In certain embodiments of the combination treatments described herein the compounds of Formula (I)-(XIX), or a pharmaceutically acceptable salts or solvates thereof, are modulators of ITPKB activity. In certain embodiments of the combination treatments described herein the compounds of Formula (I)-(XIX), or a pharmaceutically acceptable salts or solvates thereof, are inhibitors of ITPKB activity.

In certain embodiments of the combination therapies described herein, the compounds of Formula (I)-(XIX) described herein, or a pharmaceutically acceptable salts or solvates thereof, and the additional therapeutics agent(s) act additively. In certain embodiments of the combination therapies described herein, the compounds of Formula (I)-(XIX) described herein, or a pharmaceutically acceptable salts or solvates thereof, and the additional therapeutics agent(s) act synergistically.

In other embodiments, a compound of Formula (I)-(XIX) described herein, or a pharmaceutically acceptable salts or solvates thereof, or a pharmaceutical composition containing a compound of Formula (I), is administered to a patient who has not previously undergone or is not currently undergoing treatment with another therapeutic agent.

The additional therapeutic agents used in combination with at least one compound of Formula (I)-(XIX) described herein, or a pharmaceutically acceptable salt or solvate thereof, include, but are not limited to anti-inflammatory agents, immunomodulatory agents and tumour necrosis factor alpha (TNF-α) inhibitors.

The anti-inflammatory agents used in combination with at least one compound of Formula (I) described herein, or a pharmaceutically acceptable salt or solvate thereof, include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide, leukotriene antagonists including, but not limited to, zileuton, aurothioglucose, gold sodium thiomalate and auranofin, steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide and other anti-inflammatory agents including, but not limited to, methotrexate, colchicine, allopurinol, probenecid, thalidomide or a derivative thereof, 5-aminosalicylic acid, retinoid, dithranol or calcipotriol, sulfinpyrazone and benzbromarone.

The immunomodulatory agents used in combination with at least one compound of Formula (I) described herein, or a pharmaceutically acceptable salt or solvate thereof, include, but are not limited to, azathioprine, tacrolimus, cyclosporine, antimalarials, methothrexate, leflunomide, corticosteroids, cyclophosphamide, cyclosporin A, cyclosporin G, mycophenolate mofetil, ascomycin, rapamycin (sirolimus), FK-506, mizoribine, 15-deoxyspergualin, brequinar, mycophenolic acid, malononitriloamindes (such as, by way of example only, leflunamide), CTLA41g, T cell receptor modulators, and cytokine receptor modulators, peptide mimetics, and antibodies (such as, by way of example only, human, humanized, chimeric, monoclonal, polyclonal, Fvs, ScFvs, Fab or F(ab)2 fragments or epitope binding fragments), nucleic acid molecules (such as, by way of example only, antisense nucleic acid molecules and triple helices), small molecules, organic compounds, and inorganic compounds. Examples of monoclonal antibodies include, but are not limited to, monoclonal antibodies for leukocyte receptors such as, by way of example only MHC, CD2, CD3, CD4, CD7, CD25, CD28, B7, CD45, CD58 or their ligands. Examples of T cell receptor modulators include, but are not limited to, anti-T cell receptor antibodies (such as, by way of example only, anti-CD4 antibodies (such as, by way of example only, cM-T412 (Boehringer), IDEC-CE9.1™ (IDEC and SKB), mAB 4162W94, Orthoclone and OKTcdr4a (Janssen-Cilag)), anti-CD3 antibodies (such as, by way of example only, Nuvion (Product Design Labs), OKT3 (Johnson & Johnson), or Rituxan (IDEC)), anti-CD5 antibodies (such as, by way of example only, an anti-CD5 ricin-linked immunoconjugate), anti-CD7 antibodies (such as, by way of example only, CHH-380 (Novartis)), anti-CD8 antibodies, anti-CD40 ligand monoclonal antibodies (such as, by way of example only, IDEC-131 (IDEC)), anti-CD52 antibodies (such as, by way of example only, CAMPATH 1H (Ilex)), anti-CD2 antibodies, anti-CD11a antibodies (such as, by way of example only, Xanelim (Genentech)), anti-B7 antibodies (such as, by way of example only, IDEC-114 (IDEC)), CTLA4-immunoglobulin, toll-like receptor (TLR) modulators. Examples of cytokine receptor modulators include, but are not limited to, soluble cytokine receptors (such as, by way of example only, the extracellular domain of a TNF-α receptor or a fragment thereof, the extracellular domain of an IL-1 receptor or a fragment thereof, and the extracellular domain of an IL-6 receptor or a fragment thereof), cytokines or fragments thereof (such as, by way of example only, interleukin (IL)-2, IL-3, IL-4, IL-5, IL-6, IL-7, IL-8, IL-9, IL-10, IL-11, IL-12, IL-15, TNF-.alpha., interferon (IFN)-, IFN-, IFN-, and GM-CSF), anti-cytokine receptor antibodies (such as, by way of example only, anti-IFN receptor antibodies, anti-IL-2 receptor antibodies (such as, by way of example only, Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (such as, by way of example only, anti-IFN antibodies, anti-TNF-antibodies, anti-IL-1 antibodies, anti-IL-6 antibodies, anti-IL-8 antibodies (such as, by way of example only, ABX-IL-8 (Abgenix)), and anti-IL-12 antibodies).

In certain embodiments, the additional thereapeutic agent(s) used in the combination therapies described herein include, but are not limited to, agents such as tumour necrosis factor alpha (TNF-α) inhibitors (such as anti-TNF monoclonal antibodies (by way of example only, Remicade, CDP-870 and adalimumab) and TNF receptor immunoglobulin molecules (by way of example only, Enbrel, Remicade, and Humira)); non-selective cyclo-oxygenase COX-1/COX-2 inhibitors (by way of example only, piroxicam, diclofenac, propionic acids such as naproxen, flubiprofen, fenoprofen, ketoprofen and ibuprofen, fenamates such as mefenamic acid, indomethacin, sulindac, azapropazone, pyrazolones such as phenylbutazone, salicylates such as aspirin), COX-2 inhibitors (by way of example only, meloxicam, celecoxib, rofecoxib, valdecoxib, lumarocoxib, parecoxib and etoricoxib); glucocorticosteroids; methotrexate, lefunomide; hydroxychloroquine, d-penicillamine, auranofin or other parenteral or oral gold preparations.

Treatment of Diseases Associated with ITPKB Activity

Compounds of Formula (I)-(XIX), pharmaceutically acceptable salts, solvates, N-oxides, prodrugs and isomers thereof, pharmaceutical compositions, and combination therapies provided herein are modulators of ITPKB activity, and are used in the treatment and/or prevention of diseases and/or disorders in which aberrant, abnormal or deregulated activity of IPTKB contributes to the pathology and/or symptomology of such diseases and/or disorders.

In certain embodiments, compounds of Formula (I)-(XIX), pharmaceutically acceptable salts, solvates, N-oxides, prodrugs and isomers thereof, pharmaceutical compositions, and combination therapies provided herein are inhibitors of ITPKB activity, and are used in the treatment and/or prevention of diseases and/or disorders in which aberrant, abnormal or deregulated activity of IPTKB contributes to the pathology and/or symptomology of such diseases and/or disorders.

In certain embodiments, compounds of Formula (I)-(XIX), pharmaceutically acceptable salts, solvates, N-oxides, prodrugs and isomers thereof, pharmaceutical compositions, and combination therapies provided herein are modulators of the cellular level/cellular concentration of IPTKB kinase, wherein such compounds, pharmaceutically acceptable salts, solvates, N-oxides, prodrugs and isomers, pharmaceutical compositions, and combination therapies modulate the ITPKb gene expressing the ITPKB kinase. In certain embodiments, such genes are down regulated by compounds of Formula (I)-(XIX), pharmaceutically acceptable salts, solvates, N-oxides, prodrugs and isomers thereof, pharmaceutical compositions, and combination therapies thereby down regulating the cellular level/cellular concentration of IPTKB kinase.

In certain embodiments, such diseases and/or disorders associated with aberrant, abnormal or deregulated activity of IPTKB are diseases and/or disorders associated with or mediated by abnormal B-cell proliferation, differentiation and activation. Such diseases and/or disorders include, but are not limited to, B-cell lymphoma, chronic transplant rejection, immune-mediated disease, autoimmune mediated diseases, and anaphylaxis and many complement mediated diseases. Such immune mediated disorders include, but are not limited to, allergy and psoriasis. Such autoimmune mediated disorders include, but are not limited to, rheumatoid arthritis (RA), systematic lupus erythematosus (SLE), hemolytic anemia, lupus, primary binary cirrhosis (PBC) and idiopathic thrombocytopenic purpura (ITP). Such allergy disorders include, but are not limited to, respiratory diseases and dermatolgical disorders. Respiratory diseases include but are not limited to, asthma, rhinitis, COPD, asthma, bronchial asthma, allergic asthma, intrinsic asthma, extrinsic asthma, exercise-induced asthma, drug-induced asthma (including aspirin and NSAID-induced) and dust-induced asthma, chronic obstructive pulmonary disease (COPD); bronchitis, acute and chronic rhinitis including rhinitis medicamentosa, and vasomotor rhinitis, and perennial and seasonal allergic rhinitis including rhinitis nervosa (hay fever). Dermatological diseases and/or disorders include, but are not limited to, dermatitis and eczema such as, by way of example only, atopic dermatitis, seborrhoeic dermatitis (Dandruff, Cradle cap), diaper rash, urushiol-induced contact dermatitis, contact dermatitis, erythroderma, lichen simplex chronicus, prurigo nodularis, itch, pruritus ani, nummular dermatitis, dyshidrosis and pityriasis alba.

In certain embodiments, compounds of Formula (I)-(XIX), pharmaceutically acceptable salts, solvates, N-oxides, prodrugs and isomers thereof, pharmaceutical compositions, and combination therapies provided herein are inhibitors of ITPKB kinase activity and are thereby inhibitors of B-cell proliferation, differentiation and activation. Therefore, in certain embodiments, such compounds of Formula (I)-(XIX), pharmaceutically acceptable salts, solvates, N-oxides, prodrugs and isomers thereof, pharmaceutical compositions, and combination therapies provided herein are useful in treating and/or preventing diseases and/or disorders associated with or mediated by abnormal B-cell proliferation, differentiation and activation including, but not limited to, those diseases and/or disorders described herein.

In certain embodiments, the compounds of Formula (I)-(XIX), pharmaceutically acceptable salts, solvates, N-oxides, prodrugs and isomers thereof, pharmaceutical compositions, and/or combinations provided herein are used in the treatment and/or prevention of respiratory diseases and/or disorders including, but not limited to, asthma, bronchial asthma, allergic asthma, intrinsic asthma, extrinsic asthma, exercise-induced asthma, drug-induced asthma (including aspirin and NSAID-induced) and dust-induced asthma, chronic obstructive pulmonary disease (COPD); bronchitis, acute and chronic rhinitis including rhinitis medicamentosa, and vasomotor rhinitis; perennial and seasonal allergic rhinitis including rhinitis nervosa (hay fever).

In certain embodiments, the compounds of Formula (I)-(XIX), pharmaceutically acceptable salts, solvates, N-oxides, prodrugs and isomers thereof, pharmaceutical compositions, and/or combinations provided herein are used in the treatment and/or prevention of dermatological disorders including, but not limited to, psoriasis, dermatitis, eczema, atopic dermatitis, contact dermatitis, urushiol-induced contact dermatitis, eczematous dermatoses, and delayed-type hypersensitivity reactions; phyto- and photodermatitis; seborrhoeic dermatitis, dermatitis herpetiformis, lichen simplex chronicus, lichen planus, lichen sclerosus et atrophica, discoid lupus erythematosus, diaper rash, erythroderma, prurigo nodularis, itch, pruritus ani, nummular dermatitis, dyshidrosis and pityriasis alba.

The compounds of Formula (I)-(XIX), pharmaceutically acceptable salts, solvates, N-oxides, prodrugs and isomers thereof, pharmaceutical compositions, and combination therapies provided herein are effective agents to treat and/or prevent diseases and/or disorders associated with or mediated by abnormal B-cell proliferation, differentiation and activation. The compounds of Formula (I)-(XIX), pharmaceutically acceptable salts, solvates, N-oxides, prodrugs and isomers thereof, pharmaceutical compositions, and combination therapies provided herein prevent de-novo antibody responses to both T cell-dependent and T cell-independent antigens, and thereby provide a novel treatment for B-cell mediated autoimmune diseases.

In addition to treating diseases and/or disorders associated with or mediated by abnormal B cell proliferation, the ITPKB inhibitors provided herein are also useful for preventing or modulating the development of such diseases and/or disorders in a subject (including human and animals such as other mammals) suspected of being, or known to be, prone to such diseases or disorders.

In certain embodiments, the compounds of Formula (I)-(XIX), pharmaceutically acceptable salts, solvates, N-oxides, prodrugs and isomers thereof, and pharmaceutical compositions and combination therapies provided herein are used as immunosuppressant agents to treat and/or prevent rheumatoid arthritis (RA), multiple sclerosis (MS), systemic lupus erythematosus (SLE), immune thrombocytopenic purpura (ITP), hemolytic anemia and transplant rejection.

By inhibiting B-cell proliferation, activation and development, the ITPKb inhibitors provided herein are useful in various therapeutic applications, and pharmacological inhibition of ITPKB provides a means to inhibit B-cell malfunction in pathological settings. The B-cell modulators employed in the therapeutic applications provided herein include, but are not limited to, the specific ITPKB-inhibitors described in the Examples and tables, infra.

Compounds of Formula (I)-(XIX), pharmaceutically acceptable salts, solvates, N-oxides, prodrugs and isomers thereof, pharmaceutical compositions, and combination therapies provided herein are used in methods for modulating ITPKB activity in a subject (human or other mammal) for the treatment and/or prevention of diseases and/or disorders associated with or mediated by aberrant, abnormal or deregulated ITPKB activity. In certain embodiments, such methods include administering to a subject a compound of Formula (I)-(XIX), or a pharmaceutical composition containing a compound of Formula (I)-(XIX), in an effective amount to modulate the kinase activity or cellular level/cellular concentration of ITPKB (such as demonstrated by the in vitro assays described, infra); thereby modulating B lymphocyte differentiation and function in a subject. In certain embodiments, the compound of Formulas (I)-(XIX) down-regulate the cellular level of the ITPKB molecule, while in other embodiments the compound of Formulas (I)-(XIX) inhibit the kinase activity of ITPKB.

Compounds of Formula (I)-(XIX), pharmaceutically acceptable salts, solvates, N-oxides, prodrugs and isomers thereof, pharmaceutical compositions, and combination therapies provided herein are used in methods for modulating B lymphocyte development and function in a subject (human or other mammal) for the treatment and/or prevention of diseases and/or disorders associated with or mediated by abnormal B-cell proliferation, differentiation and activation including, but not limited to, those diseases and/or disorders described herein. In certain embodiments, such methods include administering to a subject a compound of Formula (I)-(XIX), or a pharmaceutical composition containing a compound of Formula (I)-(XIX), in an effective amount to modulate the kinase activity or cellular level/cellular concentration of ITPKB (such as demonstrated by the in vitro assays described, infra); thereby modulating B lymphocyte differentiation and function in a subject. In certain embodiments, the compound of Formulas (I)-(XIX) down-regulate the cellular level of the ITPKB molecule, while in other embodiments the compound of Formulas (I)-(XIX) inhibit the kinase activity of ITPKB.

In certain embodiments, the methods for the treatment of a subject suffering from a disease and/or disorder associated with aberrant, abnormal or deregulated ITPKB activity include administering to the subject an effective amount of a compound of Formula (I)-(XIX) or a pharmaceutically acceptable salt, solvate thereof, either alone or as part of a pharmaceutical composition as described herein.

In certain embodiments, are methods for treating a disease or disorder where modulation of B lymphocyte development and function is implicated, wherein such methods include administering to a system or subject in need of such treatment an effective amount of a compound of Formula (I)-(XIX), or pharmaceutically acceptable salts or pharmaceutical compositions thereof, thereby treating the disease or disorder including, but not limited to, those diseases and/or disorders described herein.

In certain embodiments, are methods for treating a cell-proliferative condition, wherein such methods include administering to a system or subject in need of such treatment an effective amount of a compound of Formula (I)-(XIX), or pharmaceutically acceptable salts or pharmaceutical compositions thereof, wherein the cell-proliferative condition is lymphoma. In certain embodiments the lymphoma is B-cell lymphoma.

In certain embodiments, such methods the diseases and/or disorders associated with aberrant, abnormal or deregulated IPTKB activity are diseases and/or disorders associated with or mediated by abnormal B-cell proliferation, differentiation and activation. Such diseases and/or disorders associated with or mediated by abnormal B-cell proliferation, differentiation and activation include, but are not limited to, B-cell lymphoma, chronic transplant rejection, immune-mediated disease, autoimmune mediated diseases, and anaphylaxis and many complement mediated diseases. Such immune mediated disorders include, but are not limited to, allergy and psoriasis. Such autoimmune mediated disorders include, but are not limited to, rheumatoid arthritis (RA), systematic lupus erythematosus (SLE), hemolytic anemia, lupus, primary binary cirrhosis (PBC) and idiopathic thrombocytopenic purpura (ITP). Such allergy disorders include, but are not limited to, respiratory diseases and dermatolgical disorders. Respiratory diseases include but are not limited to, asthma, rhinitis, COPD, asthma, bronchial asthma, allergic asthma, intrinsic asthma, extrinsic asthma, exercise-induced asthma, drug-induced asthma (including aspirin and NSAID-induced) and dust-induced asthma, chronic obstructive pulmonary disease (COPD); bronchitis, acute and chronic rhinitis including rhinitis medicamentosa, and vasomotor rhinitis, and perennial and seasonal allergic rhinitis including rhinitis nervosa (hay fever). Dermatological diseases and/or disorders include, but are not limited to, dermatitis and eczema such as, by way of example only, atopic dermatitis, seborrhoeic dermatitis (Dandruff, Cradle cap), diaper rash, urushiol-induced contact dermatitis, contact dermatitis, erythroderma, lichen simplex chronicus, prurigo nodularis, itch, pruritus ani, nummular dermatitis, dyshidrosis and pityriasis alba.

In certain embodiments, a compound of Formula (I)-(XIX), or a pharmaceutically acceptable salt or solvate thereof, is used in the preparation of a medicament for the treatment of a disease or disorder associated with aberrant, abnormal or deregulated ITPKB activity. In other embodiments, a compound of Formula (I)-(XIX), or a pharmaceutically acceptable salt or solvate thereof, is used in the treatment of a disease or disorder associated with aberrant, abnormal or deregulated ITPKB activity.

In a further embodiment, pharmaceutical compositions comprising at least one compound of Formula (I)-(XIX) are adapted for oral administration for the treatment of immune-mediated diseases. In a further embodiment, pharmaceutical compositions comprising at least one compound of Formula (I)-(XIX) are adapted for oral administration for the treatment of autoimmune-mediated diseases. In a further embodiment, the pharmaceutical compositions comprising at least one compound of Formula (I)-(XIX) are adapted for oral administration for the treatment of rheumatoid arthritis. In a further embodiment, pharmaceutical compositions comprising at least one compound of Formula (I)-(XIX) are adapted for oral administration for the treatment of systematic lupus erythematosus (SLE). In a further embodiment, pharmaceutical compositions comprising at least one compound of Formula (I)-(XIX) are adapted for oral administration for the treatment of primary binary cirrhosis (PBC). In a further embodiment, pharmaceutical compositions comprising at least one compound of Formula (I)-(XIX) are adapted for oral administration for the treatment of idiopathic thrombocytopenic purpura (ITP). In a further embodiment, pharmaceutical compositions comprising at least one compound of Formula (I)-(XIX) are adapted for oral administration for the treatment of asthma. In a further embodiment, pharmaceutical compositions comprising at least one compound of Formula (I)-(XIX) are adapted for oral administration for the treatment of rhinitis. In a further embodiment, pharmaceutical compositions comprising at least one compound of Formula (I)-(XIX) are adapted for oral administration for the treatment of COPD.

In a further embodiment, pharmaceutical compositions comprising at least one compound of Formula (I)-(XIX)) are adapted for topical administration for the treatment of dermatological diseases and/or disorders associated with ITPKB activity. In a further embodiment, pharmaceutical compositions comprising at least one compound of Formula (I)-(XIX) are adapted for topical administration for the treatment of dermatitis.

In certain embodiments, the system or subject used in the methods provided herein are cell or tissue systems. In certain embodiments, the system or subject used in the methods provided herein are human or animal subjects.

In accordance with the foregoing, provided herein are methods for preventing, treating and/or ameliorating the condition of any of the diseases or disorders described above in a subject in need of such treatment, which method comprises administering to said subject a therapeutically effective amount of a compound of Formula (I)-(XIX), or a pharmaceutically acceptable salt thereof. For any of the methods and uses provided herein, the required dosage will vary depending on the mode of administration, the particular condition to be treated and the effect desired.

### Kits

Also provided herein are pharmaceutical packs or kits that include one or more containers containing a compound of Formula (I)-(XIX) useful for the treatment or prevention of a disease or disorder associated with ITPKB activity. In other embodiments, such pharmaceutical packs or kits include one or more containers containing a compound of Formula (I)-(XIX) useful for the treatment or prevention of a disease or disorder associated with ITPKB activity and one or more containers containing an additional therapeutic agent, including but not limited to those listed above. In certain embodiments, such pharmaceutical packs or kits optionally include instructions for its administration of a compound of Formula (I)-(XIX) as disclosed herein.

### EXAMPLES

The following examples are offered to illustrate, but not to limit, the compounds of Formula (I) provided herein, and the preparation of such compounds.

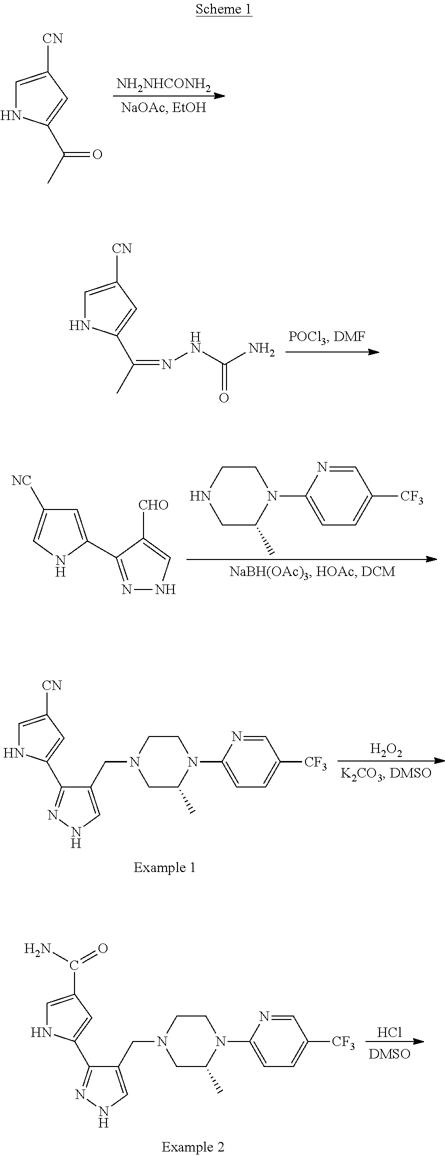

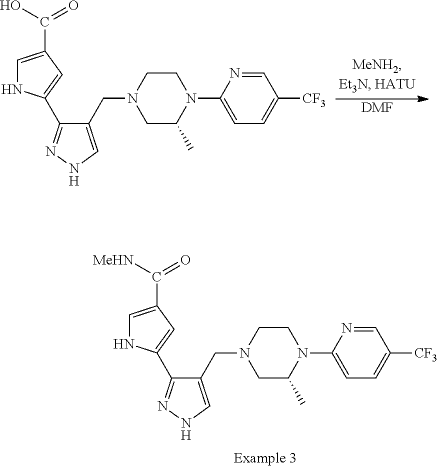

### Example 1

### Preparation of (R)-5-(4-((3-methyl-4-(5-(trifluoromethyl)pyridin-2-yl)piperazin-1-yl)methyl)-1H-pyrazol-3-yl)-1H-pyrrole-3-carbonitrile

(R)-5-(4-((3-methyl-4-(5-(trifluoromethyl)pyridin-2-yl)piperazin-1-yl)methyl)-1H-pyrazol-3-yl)-1H-pyrrole-3-carbonitrile is synthesized in three steps as shown in scheme 1.

In step 1-1 a solution of sodium acetate trihydrate (8.3 g, 61.2 mmol) in 80 ml water is added to a mixture of 5-acetyl-1H-pyrrole-3-carbonitrile (2.05 g, 15.3 mmol) and semicarbazide HCl salt (4.09 g, 37 mmol) in 20 ml of ethanol. This mixture is refluxed for 6 hours until the reaction is completed and the mixture is then cooled to room temperature and the total volume is reduced to 40 ml by rotary evaporator. The white solid precipitate formed is collected by vacuum filtration, washed with water and air-dried to give 2-(1-(4-cyano-1H-pyrrol-2-yl)ethylidene)hydrazinecarboxamide (m/z [M⁺+1] 192.1), which is used in step 1-2 without further purification.

In step 1-2 a round-bottom flask containing DMF (6.1 ml, 79 mmol) is cooled with an ice bath and upon cooling phosphoryl chloride (6.0 g, 39 mmol) is added dropwise into the flask. The solution is stirred for 10 minutes and then 2-(1-(4-cyano-1H-pyrrol-2-yl)ethylidene)hydrazinecarboxamide from step 1-1 (2.5 g, 13 mmol) is added portion-wise. The solution is then warmed to 75° C. and kept at this temperature for 2 hours. The solution is then cooled to 0° C., and ice-water is added. The solution is adjusted to pH 7 with 1N NaOH, and is extracted with ethyl acetate (10 ml×3). The organic layers are combined, dried, and concentrated. The residue is purified with silica gel chromatography (eluted with hexane-ethyl acetate) to give 5-(4-formyl-1H-pyrazol-3-yl)-1H-pyrrole-3-carbonitrile as a light yellow solid. ¹H NMR (DMSO-d₆) δ 7.29 (s, 1H), 7.69 (s, 1H), 8.66 (s, 1H), 9.92 (s, 1H), 12.31 (bs, 1H); m/z [M⁺+1] 187.1.

In step 1-3 NaBH(OAc)₃ (0.45 g, 2.1 mmol) is added to a suspension of 5-(4-formyl-1H-pyrazol-3-yl)-1H-pyrrole-3-carbonitrile from step 1-2 (0.19 g, 1.0 mmol), (R)-2-methyl-1-(5-(trifluoromethyl)pyridin-2-yl)piperazine (0.26 g, 1.0 mmol) and acetic acid (0.13 g, 2.1 mmol) in 40 ml of CH₂Cl₂. The mixture is stirred at 45° C. for 18 hours until the reaction is completed, and then saturated sodium carbonate solution is added to adjust to pH 12. The mixture is extracted with CH₂Cl₂ (40 ml×3) and the organic layers are combined, dried, and concentrated. The residue is purified with silica gel chromatography (eluted with hexane-ethyl acetate) to give (R)-5-(4-((3-methyl-4-(5-(trifluoromethyl)pyridin-2-yl)piperazin-1-yl)methyl)-1H-pyrazol-3-yl)-1H-pyrrole-3-carbonitrile as a colorless solid. ¹H NMR (DMSO-d₆) δ 1.16 (d, 3H, J=6.8 Hz), 2.01 (t, 1H, J=9.6 Hz), 2.23 (d, 1H, J=8.4 Hz), 2.89 (d, 1H, J=12 Hz), 2.98 (d, 1H, J=10.4 Hz), 3.08 (t, 1H, J=12 Hz), 3.45 (m, 2H), 4.21 (d, 1H, J=12 Hz), 4.65 (b, 1H), 6.83 (s, 1H), 6.91 (d, 1H, J=8.8 Hz), 7.63 (s, 1H), 7.75 (s, 1H), 7.80 (d, 1H, J=8.8 Hz), 8.42 (s, 1H), 12.57 (bs, 1H), 12.89 (bs, 1H); m/z [M⁺+1] 416.2.

### Example 2

### Preparation of (R)-5-(4-((3-methyl-4-(5-(trifluoromethyl)pyridin-2-yl)piperazin-1-yl)methyl)-1H-pyrazol-3-yl)-1H-pyrrole-3-carboxamide

(R)-5-(4-((3-methyl-4-(5-(trifluoromethyl)pyridin-2-yl)piperazin-1-yl)methyl)-1H-pyrazol-3-yl)-1H-pyrrole-3-carboxamide is synthesized from (R)-5-(4-((3-methyl-4-(5-(trifluoromethyl)pyridin-2-yl)piperazin-1-yl)methyl)-1H-pyrazol-3-yl)-1H-pyrrole-3-carbonitrile (Example 1) as shown in scheme 1.

To a solution of (R)-5-(4-((3-methyl-4-(5-(trifluoromethyl)pyridin-2-yl)piperazin-1-yl)methyl)-1H-pyrazol-3-yl)-1H-pyrrole-3-carbonitrile (100 mg, 0.24 mmol) in 4 ml of DMSO is added K₂CO₃ (128 mg, 0.92 mmol) and 30% H₂O₂ (0.28 ml). The reaction is heated at 40° C. for 18 hours, then cooled to room temperature, diluted with water and extracted with EtOAc (20 ml×3). The organic layers are combined, washed with water, dried with anhydrous Na₂SO₄ and concentrated to give the title compound as a white solid, which is further purified by HPLC (C₁₈ column, eluted with CH₃CN/H₂O with 0.035% TFA). The factions containing product are combined, neutralized with sodium carbonate, and extracted with ethyl acetate. The organic layers are combined, dried, and concentrated to give (R)-5-(4-((3-methyl-4-(5-(trifluoromethyl)pyridin-2-yl)piperazin-1-yl)methyl)-1H-pyrazol-3-yl)-1H-pyrrole-3-carboxamide. ¹H NMR (DMSO-d₆) δ 1.20 (d, 3H, J=6.8 Hz), 2.02 (t, 1H, J=8.0 Hz), 2.27 (d, 1H, J=8.0 Hz), 2.94 (d, 1H, J=11.6 Hz), 3.00 (d, 1H, J=11.2 Hz), 3.09 (t, 1H, J=13.2 Hz), 3.45 (m, 2H), 4.20 (d, 1H, J=11.6 Hz), 4.65 (b, 1H), 6.68 (bs, 1H), 6.79 (s, 1H), 6.92 (d, 1H, J=8.8 Hz), 7.35 (s, 1H), 7.44 (b, 1H), 7.70 (s, 1H), 7.81 (d, 1H, J=8.8 Hz), 8.42 (s, 1H), 12.02 (bs, 1H), 12.72 (bs, 1H); m/z [M⁺+1] 434.2.

### Example 3

### Preparation of (R)—N-methyl-5-(4-((3-methyl-4-(5-(trifluoromethyl)pyridin-2-yl)piperazin-1-yl)methyl)-1H-pyrazol-3-yl)-1H-pyrrole-3-carboxamide

(R)—N-methyl-5-(4-((3-methyl-4-(5-(trifluoromethyl)pyridin-2-yl)piperazin-1-yl)methyl)-1H-pyrazol-3-yl)-1H-pyrrole-3-carboxamide is synthesized from (R)-5-(4-((3-methyl-4-(5-(trifluoromethyl)pyridin-2-yl)piperazin-1-yl)methyl)-1H-pyrazol-3-yl)-1H-pyrrole-3-carboxamide (Example 2) in two steps as shown in scheme 1.

In step 3-1 4 ml of 2N HCl is added to a solution of (90 mg, 0.21 mmol) (R)-5-(4-((3-methyl-4-(5-(trifluoromethyl)pyridin-2-yl)piperazin-1-yl)methyl)-1H-pyrazol-3-yl)-1H-pyrrole-3-carboxamide in 1 ml of DMSO. The mixture is heated at 90° C. for 8 hours and then cooled to room temperature, concentrated, and purified by HPLC (C₁₈ column, eluted with CH₃CN/H₂O with 0.035% TFA). The factions containing product are combined and concentrated to give (R)-5-(4-((3-methyl-4-(5-(trifluoromethyl)pyridin-2-yl)piperazin-1-yl)methyl)-1H-pyrazol-3-yl)-1H-pyrrole-3-carboxylic acid, m/z [M⁺+1] 435.2.

In step 3-2 HATU (13.1 mg, 0.035 mmol) is added to a solution of (R)-5-(4-((3-methyl-4-(5-(trifluoromethyl)pyridin-2-yl)piperazin-1-yl)methyl)-1H-pyrazol-3-yl)-1H-pyrrole-3-carboxylic acid (10 mg, 0.023 mmol) and triethyl amine (7 mg, 0.069 mmol) in DMF. The mixture is stirred for 10 minutes before methyl amine HCl salt (2.3 mg, 0.034 mmol) is added. After 2 hours, the mixture is concentrated and the residue is purified by HPLC (C₁₈ column, eluted with CH₃CN/H₂O with 0.035% TFA). The factions containing product are combined and lyophilized to give the TFA salt of the title compound as colorless oil, m/z [M⁺+1] 448.2.

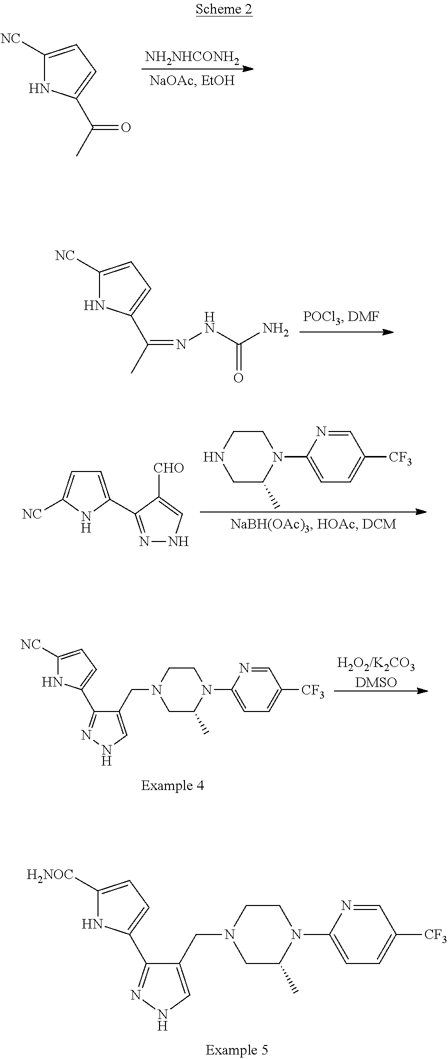

### Example 4

### Preparation of (R)-5-(4-((3-methyl-4-(5-(trifluoromethyl)pyridin-2-yl)piperazin-1-yl)methyl)-1H-pyrazol-3-yl)-1H-pyrrole-2-carbonitrile

(R)-5-(4-((3-methyl-4-(5-(trifluoromethyl)pyridin-2-yl)piperazin-1-yl)methyl)-1H-pyrazol-3-yl)-1H-pyrrole-2-carbonitrile is synthesized in three steps as shown in scheme 2 from 5-acetyl-1H-pyrrole-2-carbonitrile.

In step 4-1 a solution of sodium acetate trihydrate (8.3 g, 61 2 mmol) in 80 ml water is added to a mixture of 5-acetyl-1H-pyrrole-2-carbonitrile (2.05 g, 15.3 mmol) and semicarbazide HCl salt (4.09 g, 37 mmol) in 20 ml of ethanol. This mixture is refluxed for 6 hours until the reaction is completed and the mixture is then cooled to room temperature and the total volume is reduced to 40 ml by rotary evaporator. The solid precipitate formed is collected by vacuum filtration, washed with water and air-dried to give 2-(1-(5-cyano-1H-pyrrol-2-yl)ethylidene)hydrazinecarboxamide (m/z [M⁺+1] 192.1), which is used in step 4-2.

In step 4-2 a round-bottom flask containing DMF (6.1 ml, 79 mmol) is cooled with an ice bath and upon cooling phosphoryl chloride (6.0 g, 39 mmol) is added dropwise into the flask. The solution is stirred for 10 minutes and then 2-(1-(5-cyano-1H-pyrrol-2-yl)ethylidene)hydrazinecarboxamide from step 4-1 (2.5 g, 13 mmol) is added portion-wise. The solution is then warmed to 75° C. and kept at this temperature for 2 hours. The solution is then cooled to 0° C., and ice-water is added. The solution is adjusted to pH 7 with 1N NaOH, and is extracted with ethyl acetate (10 ml×3). The organic layers are combined, dried, and concentrated. The residue is purified with silica gel chromatography (eluted with hexane-ethyl acetate) to give 5-(4-formyl-1H-pyrazol-3-yl)-1H-pyrrole-2-carbonitrile as a yellow solid.¹H NMR (DMSO-d₆) δ 7.02 (m, 2H), 8.67 (s, 1H), 9.94 (s, 1H), 12.72 (bs, 1H); m/z [M⁺+1] 1187.1.

In step 4-3 NaBH(OAc)₃ (0.45 g, 2.1 mmol) is added to a suspension of 5-(4-formyl-1H-pyrazol-3-yl)-1H-pyrrole-2-carbonitrile from step 4-2 (0.19 g, 1 0 mmol), (R)-2-methyl-1-(5-(trifluoromethyl)pyridin-2-yl)piperazine (0.26 g, 1.0 mmol) and acetic acid (0.13 g, 2.1 mmol) in 40 ml of CH₂Cl₂. The mixture is stirred at 45° C. for 18 hours until the reaction is completed, and then saturated sodium carbonate solution is added to adjust to pH 12. The mixture is extracted with CH₂Cl₂ (40 ml×3) and the organic layers are combined, dried, and concentrated. The residue is purified with silica gel chromatography (eluted with hexane-ethyl acetate) to give (R)-5-(4-((3-methyl-4-(5-(trifluoromethyl)pyridin-2-yl)piperazin-1-yl)methyl)-1H-pyrazol-3-yl)-1H-pyrrole-2-carbonitrile is colorless solid. ¹H NMR (DMSO-d₆) δ 1.14 (d, 3H, J=6.8 Hz), 2.06 (t, 1H, J=12 Hz), 2.24 (d, 1H, J=8.0 Hz), 2.90 (d, 1H, J=12 Hz), 3.06 (m, 1H), 3.50 (m, 2H), 4.26 (d, 1H, J=12 Hz), 4.65 (b, 1H), 6.61 (d, 1H, J=3.6 Hz), 6.92 (d, 1H, J=9.6 Hz), 6.99(d, 1H, J=9.6 Hz), 7.89 (s, 2H), 7.82 (s, 1H), 8.42 (s, 1H), 12.97 (bs, 1H); m/z [M⁺+1] 416.2.

### Example 5

### Preparation of (R)-5-(4-((3-methyl-4-(5-(trifluoromethyl)pyridin-2-yl)piperazin-1-yl)methyl)-1H-pyrazol-3-yl)-1H-pyrrole-2-carboxamide

(R)-5-(4-((3-methyl-4-(5-(trifluoromethyl)pyridin-2-yl)piperazin-1-yl)methyl)-1H-pyrazol-3-yl)-1H-pyrrole-2-carboxamide is prepared from (R)-5-(4-((3-methyl-4-(5-(trifluoromethyl)pyridin-2-yl)piperazin-1-yl)methyl)-1H-pyrazol-3-yl)-1H-pyrrole-2-carbonitrile as shown in scheme 2 using the method as described for the synthesis of Example 2.

To a solution of (R)-5-(4-((3-methyl-4-(5-(trifluoromethyl)pyridin-2-yl)piperazin-1-yl)methyl)-1H-pyrazol-3-yl)-1H-pyrrole-2-carbonitrile (100 mg, 0.24 mmol) in 4 ml of DMSO is added K₂CO₃ (128 mg, 0.92 mmol) and 30% H₂O₂ (0.28 ml). The reaction is heated at 40° C. for 18 hours, then cooled to room temperature, diluted with water and extracted with EtOAc (20 ml×3). The organic layers are combined, washed with water, dried with anhydrous Na₂SO₄ and concentrated to give the title compound as a white solid, which is further purified by HPLC (C₁₈ column, eluted with CH₃CN/H₂O with 0.035% TFA). The factions containing product are combined, neutralized with sodium carbonate, and extracted with ethyl acetate. The organic layers are combined, dried, and concentrated to give (R)-5-(4-((3-methyl-4-(5-(trifluoromethyl)pyridin-2-yl)piperazin-1-yl)methyl)-1H-pyrazol-3-yl)-1H-pyrrole-2-carboxamide, m/z [M⁺+1] 434.2.

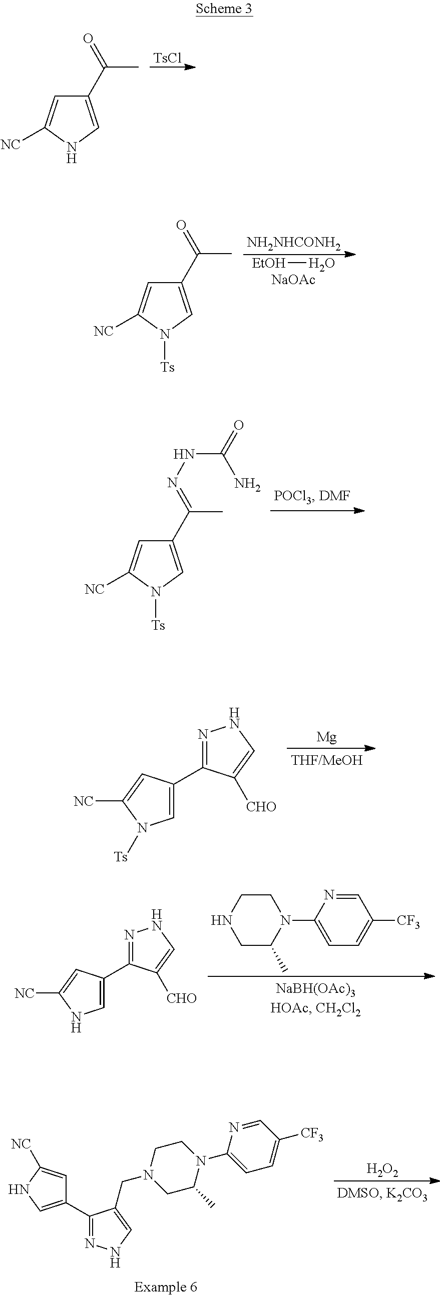

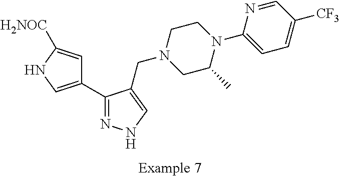

### Example 6

### Preparation of (R)-4-(4-((3-methyl-4-(5-(trifluoromethyl)pyridin-2-yl)piperazin-1-yl)methyl)-1H-pyrazol-3-yl)-1H-pyrrole-2-carbonitrile

(R)-4-(4-((3-methyl-4-(5-(trifluoromethyl)pyridin-2-yl)piperazin-1-yl)methyl)-1H-pyrazol-3-yl)-1H-pyrrole-2-carbonitrile is synthesized in five steps from 4-acetyl-1H-pyrrole-2-carbonitrile as shown in scheme 3 above.

In step 6-1 triethyl amine (10.1 g, 0.1 mol) is added to a solution of 4-acetyl-1H-pyrrole-2-carbonitrile (6.7 g, 50 mmol) in 250 ml of anhydrous acetonitrile. The mixture is cooled to 0° C. in an ice bath and then tosyl chloride (10.5 g, 55 mmol) is added in portions. The reaction is stirred at 0° C. for 1 hour and then at room temperature for 3 hours. The solvent is then removed and a saturated solution of sodium carbonate is added. The mixture is extracted with ethyl acetate (100 ml×3), and the organic layers are combined, washed with water, dried with anhydrous Na₂SO₄ and concentrated. The white solid obtained is sonicated in 20 ml of acetonitrile and filtered. The filtered solid is washed with a small amount of acetonitrile and air-dried to give the tosyl protected compound 4-acetyl-1-tosyl-1H-pyrrole-2-carbonitrile, m/z [M⁺+1] 289.2. More product is obtained by purification of the mother liquor using silica gel column chromatography (eluted with hexanes-ethyl acetate).

In step 6-2 a solution of sodium acetate trihydrate (15.1 g, 0.111 mol) in 150 ml water is added to a mixture of 4-acetyl-1-tosyl-1H-pyrrole-2-carbonitrile (8.0 g, 27.8 mmol) and semicarbazide HCl salt (6.2 g, 55.6 mmol) in 150 ml ethanol. The mixture is refluxed for 20 hours until the reaction is completed. The mixture is cooled to room temperature and the total volume is reduced to 100 ml by rotary evaporator. The white solid precipitate formed is collected by vacuum filtration. The solid is washed by water and air-dried to give 2-(1-(5-cyano-1-tosyl-1H-pyrrol-3-yl)ethylidene)hydrazinecarboxamide, m/z [M⁺+1] 346.2. It is used without further purification.

In step 6-3 a round-bottom flask charged with DMF (4.66 ml, 60 mmol) is cooled with an ice bath and phosphoryl chloride (4.6 g, 30 mmol) is then added dropwise into the flask. The solution is stirred for 10 minutes, the 2-(1-(5-cyano-1-tosyl-1H-pyrrol-3-yl)ethylidene)hydrazinecarboxamide (3.45 g, 10 mmol) is added portion-wise. The solution is then warmed to 75° C. and kept at this temperature for 2 hours before the cooling to 0° C., and adding ice-water. The solution is adjusted to pH 7 with 1N NaOH and the off-white precipitate formed is collected by vacuum filtration, washed with ethyl acetate and air-dried to give the desired intermediate 4-(4-formyl-1H-pyrazol-3-yl)-1-tosyl-1H-pyrrole-2-carbonitrile. The filtrate is extracted with ethyl acetate (50 ml×3) and the organic layers are combined, dried, and concentrated. The residue is purified with silica gel chromatography (eluted with hexane-ethyl acetate) to give more product. ¹H NMR (DMSO-d₆) δ 2.41 (s, 1H), 7.56 (d, 2H, J=8.0 Hz), 7.83 (s, 1H), 7.96 (d, 2H, J=8.0 Hz), 8.61 (s, 1H), 8.67 (s, 1H), 9.92 (s, 1H); m/z [M⁺+1] 341.1.

In step 6-4 a suspension containing 4-(4-formyl-1H-pyrazol-3-yl)-1-tosyl-1H-pyrrole-2-carbonitrile (42 mg, 0.12 mmol) and magnesium powder (42 mg, 50 mesh, 1.77 mmol) in MeOH-THF (8 ml, 3:1) is sonicated for 48 hours until the reaction is completed. It is diluted with CH₂Cl₂ and 0.5 N HCl is added until the reaction is clear. The organic layer is separated and the aqueous layer is extracted with CH₂Cl₂ two more times. The combined organic layers are washed with 1M sodium bicarbonate, dried and concentrated. The residue is purified with silica gel chromatography (eluted with hexane-ethyl acetate) to give 4-(4-formyl-1H-pyrazol-3-yl)-1H-pyrrole-2-carbonitrile, m/z [M⁺+1] 187.1.

In step 6-5 NaBH(OAc)₃ (85 mg, 0.4 mmol) is added to a suspension of 4-(4-formyl-1H-pyrazol-3-yl)-1H-pyrrole-2-carbonitrile (22 mg, 0.117 mmol), (R)-2-methyl-1-(5-(trifluoromethyl)pyridin-2-yl)piperazine (36 mg, 0.147 mmol) and acetic acid (48 mg, 0.8 mmol) in 5 ml of CH₂Cl₂. The mixture is stirred at 45° C. for 18 hours until the reaction is completed, and the mixture is then concentrated and the residue is purified by HPLC (C₁₈ column, eluted with CH₃CN/H₂O with 0.035% TFA). The factions containing product are combined, neutralized with sodium carbonate, and extracted with ethyl acetate. The organic layers are combined, dried, and concentrated to give (R)-4-(4-((3-methyl-4-(5-(trifluoromethyl)pyridin-2-yl)piperazin-1-yl)methyl)-1H-pyrazol-3-yl)-1H-pyrrole-2-carbonitrile as colorless solid. ¹H NMR (DMSO-d₆) δ 1.12 (d, 3H, J=4.2 Hz), 1.95 (t, 1H, J=11.6 Hz), 2.16 (dd, 1H, J₁=3.6 Hz, J₂=11.2 Hz), 2.82 (d, 1H, J=11.2 Hz), 2.94 (d, 1H, J=12 Hz), 2.99 (t, 1H, J=13 Hz), 3.38 (m, 2H), 4.14 (d, 1H, J=12 Hz), 4.59 (b, 1H), 6.89 (d, 1H, J=8.8 Hz), 7.28 (s, 1H), 7.56 (s, 1H), 7.78 (dd, 1H, J₁=2.0 Hz, J₂=8.8 Hz), 8.40 (s, 1H), 12.60 (bs, 1H), 12.92 (bs, 1H); m/z [M⁺+1] 416.2.

### Example 7

### Preparation of R)-4-(4-((3-methyl-4-(5-(trifluoromethyl)pyridin-2-yl)piperazin-1-yl)methyl)-1H-pyrazol-3-yl)-1H-pyrrole-2-carboxamide

(R)-4-(4-((3-methyl-4-(5-(trifluoromethyl)pyridin-2-yl)piperazin-1-yl)methyl)-1H-pyrazol-3-yl)-1H-pyrrole-2-carboxamide is synthesized from (R)-4-(4-((3-methyl-4-(5-(trifluoromethyl)pyridin-2-yl)piperazin-1-yl)methyl)-1H-pyrazol-3-yl)-1H-pyrrole-2-carbonitrile as shown in scheme 3 using the method described above for Example 2.

To a solution of (R)-4-(4-((3-methyl-4-(5-(trifluoromethyl)pyridin-2-yl)piperazin-1-yl)methyl)-1H-pyrazol-3-yl)-1H-pyrrole-2-carbonitrile (245 mg, 0.59 mmol) in 3 ml of DMSO is added K₂CO₃ (200 mg, 1.45 mmol) and 30% H₂O₂ (0.4 ml). The reaction is heated at 40° C. for 18 hours, then cooled to room temperature, diluted with water and extracted with EtOAc (20 ml×3). The organic layers are combined, washed with water, dried with anhydrous Na₂SO₄ and concentrated to give the title compound as a white solid, which is further purified by HPLC (C₁₈ column, eluted with CH₃CN/H₂O with 0.035% TFA). The factions containing product are combined, neutralized with sodium carbonate, and extracted with ethyl acetate. The organic layers are combined, dried, and concentrated to give R)-4-(4-((3-methyl-4-(5-(trifluoromethyl)pyridin-2-yl)piperazin-1-yl)methyl)-1H-pyrazol-3-yl)-1H-pyrrole-2-carboxamide, ¹H NMR ¹H NMR (DMSO-d₆) δ 1.14 (d, 3H, J=6.8 Hz), 1.98 (t, 1H, J=8.8 Hz), 2.16 (dd, 1H, J₁=3.6 Hz, J₂=10.8 Hz), 2.85 (d, 1H, J=11.6 Hz), 2.95 (d, 1H, J=8.4 Hz), 3.02 (t, 1H, J=12 Hz), 3.40 (m, 2H), 4.16 (d, 1H, J=12.4 Hz), 4.58 (b, 1H), 6.88 (d, 1H, J=9.6 Hz), 7.08 (s, 1H), 7.32 (s, 1H), 7.39 (bs, 1H), 7.55 (s, 1H), 7.77 (dd, 1H, J₁=2.4 Hz, J₂=8.8 Hz), 8.40 (s, 1H), 11.57 (bs, 1H), 12.45 (bs, 1H), 12.73 (bs, 1H); m/z [M⁺+1] 434.2.

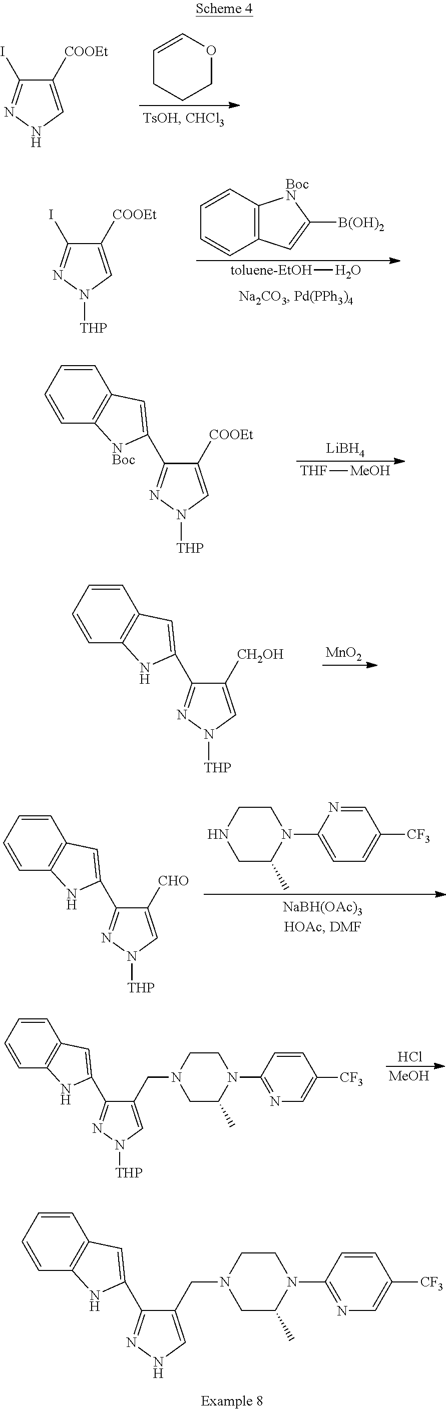

### Example 8

### Preparation of (R)-2-(4-((3-methyl-4-(5-(trifluoromethyl)pyridin-2-yl)piperazin-1-yl)methyl)-1H-pyrazol-3-yl)-1H-indole

(R)-2-(4-((3-methyl-4-(5-(trifluoromethyl)pyridin-2-yl)piperazin-1-yl)methyl)-1H-pyrazol-3-yl)-1H-indole is synthesized in six steps as shown in scheme 4 above.

In step 8-1 a solution of ethyl 3-iodo-1H-pyrazole-4-carboxylate (0.25 g, 0.94 mmol) in a mixture of CHCl₃ (12 ml) and THF (6 ml) is treated with 3,4-dihydro-2H-pyran (0.32g, 0.34 mmol) and p-toluenesulfonic acid monohydrate (12 mg) at room temperature for 18 hours. The mixture is then diluted with CH₂Cl₂ and the organic layer is separated, washed with saturated sodium bicarbonate and concentrated to give ethyl 3-iodo-1-(tetrahydro-2H-pyran-2-yl)-1H-pyrazole-4-carboxylate which is used in step 8-2 without further purification.

In step 8-2, 3 ml of 2M Na₂CO₃, 3 ml of ethanol and 6 ml of toluene to a round flask containing 1-(tert-butoxycarbonyl)-1H-indol-2-ylboronic acid (149 mg, 0.57 mmol), ethyl 3-iodo-1-(tetrahydro-2H-pyran-2-yl)-1H-pyrazole-4-carboxylate (100 mg, 0.29 mmol) and Pd(PPh₃)₄ (33 mg, 0.029 mmol). The flask is purged with argon and sealed, and the mixture is stirred at 80° C. for 18 hours, cooled to ambient temperature and then extracted with ethyl acetate. The organic layer is combined, dried and concentrated. The residue is purified by silica gel column chromatography (eluted with EtOAc/Hexane) to give tert-butyl 2-(4-(ethoxycarbonyl)-1-(tetrahydro-2H-pyran-2-yl)-1H-pyrazol-3-yl)-1H-indole-1-carboxylate, m/z [M⁺+1] 440.2.

In step 8-3, 100 mg of LiBH₄ is added to a solution of tert-butyl 2-(4-(ethoxycarbonyl)-1-(tetrahydro-2H-pyran-2-yl)-1H-pyrazol-3-yl)-1H-indole-1-carboxylate (38 mg, 0.087 mmol) in THF (5 ml) with 0.1 ml of MeOH. The reaction mixture is stirred at 70° C. for 24 hours until the ester disappears, and is then quenched with 1N HCl, and NaHCO₃ is used to adjust to pH 5. The mixture is extracted with ethyl acetate (10 ml×3), and the organic layers are combined, dried and concentrated to give (3-(1H-indol-2-yl)-1-(tetrahydro-2H-pyran-2-yl)-1H-pyrazol-4-yl)methanol, m/z [M⁺+1] 298.2, which is used in step 8-4 without purification.

In step 8-4 activated MnO₂ (263 mg, 3.03 mmol) is added to (3-(1H-indol-2-yl)-1-(tetrahydro-2H-pyran-2-yl)-1H-pyrazol-4-yl)methanol (from step 8-3) dissolved in 10 ml of CH₂Cl₂. The mixture is stirred for 12 hours in a 40° C. oil-bath and the MnO₂ is then removed by filtration. The mixture is concentrated, and purified by silica gel chromatography (eluted with hexane-ethyl acetate) to give 3-(1H-indol-2-yl)-1-(tetrahydro-2H-pyran-2-yl)-1H-pyrazole-4-carbaldehyde, m/z [M⁺+1] 296.2.

In step 8-5, NaBH(OAc)₃ (82 mg, 0.384 mmol) is added to a solution of 3-(1H-indol-2-yl)-1-(tetrahydro-2H-pyran-2-yl)-1H-pyrazole-4-carbaldehyde (19 mg, 0.064 mmol), (R)-2-methyl-1-(5-(trifluoromethyl)pyridin-2-yl)piperazine (32 mg, 0.13 mmol) and acetic acid (23 mg, 0.38 mmol) in 5 ml of DMF. The mixture is stirred for 2 hours at room temperature until the reaction is completed, then the mixture is concentrated, and a 2M Na₂CO₃ solution is added. The mixture is then extracted with ethyl acetate (10 ml×3), and the organic layers are combined, dried, and concentrated. The residue is purified with silica gel chromatography (eluted with hexane-ethyl acetate) to give 2-(4-(((R)-3-methyl-4-(5-(trifluoromethyl)pyridin-2-yl)piperazin-1-yl)methyl)-1-(tetrahydro-2H-pyran-2-yl)-1H-pyrazol-3-yl)-1H-indole, m/z [M⁺+1] 525.2.

In step 8-6, 1 ml of 5M HCl in i-PrOH is added to a solution of 2-(4-(((R)-3-methyl-4-(5-(trifluoromethyl)pyridin-2-yl)piperazin-1-yl)methyl)-1-(tetrahydro-2H-pyran-2-yl)-1H-pyrazol-3-yl)-1H-indole (19 mg, 0.036 mmol) in 10 ml of methanol. The mixture is stirred at room temperature for 2 hours and the solvent is then removed and the residue is purified by HPLC (C₁₈ column, eluted with CH₃CN/H₂O with 0.035% TFA). The factions containing product are combined and lyophilized to give the TFA salt of (R)-2-(4-((3-methyl-4-(5-(trifluoromethyl)pyridin-2-yl)piperazin-1-yl)methyl)-1H-pyrazol-3-yl)-1H-indole, ¹H NMR (DMSO-d₆) δ 1.23 (d, 3H, J=6.8 Hz), 3.13 (m, 1H), 3.25 (m, 2H), 3.49 (m, 2H), 3.8 (m, 1H), 4.51 (d, 1H, J=13.6 Hz), 4.58 (b, 1H), 4.91 (b, 1H), 6.99 (m, 3H), 7.11 (t, 1H, J=7.2 Hz), 7.44 (d, 1H, J=7.2 Hz), 7.55 (d, 1H, J=8.4 Hz), 7.88 (d, 1H, J=8.8 Hz), 8.04 (s, 1H), 8.45 (s, 1H), 9.56 (bs, 1H), 11.4 (bs, 1H); m/z [M⁺+1] 441.2.

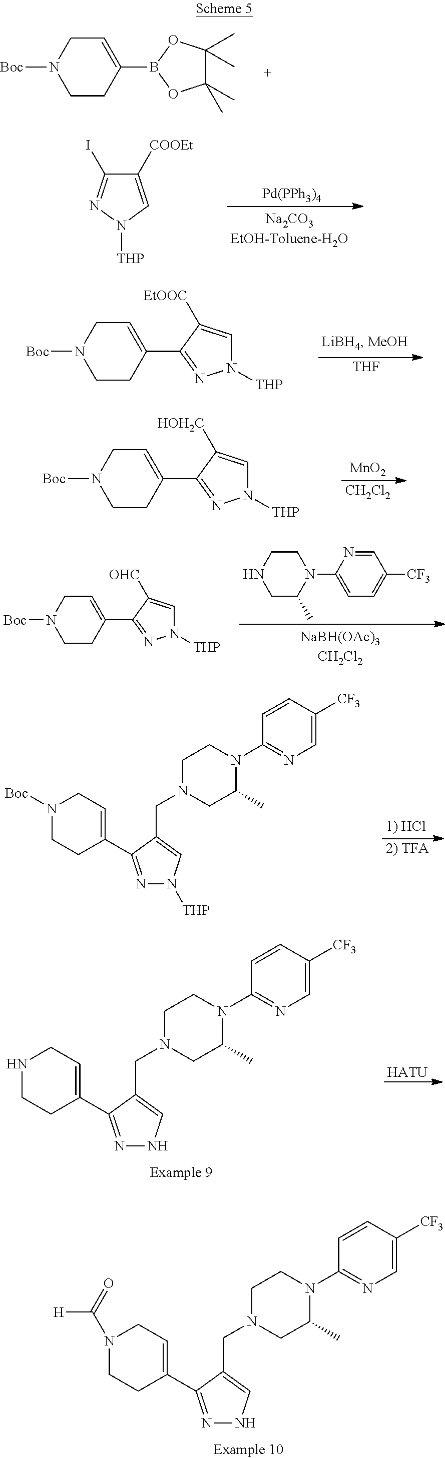

### Example 9

### Preparation of (R)-2-methyl-4-((3-(1,2,3,6-tetrahydropyridin-4-yl)-1H-pyrazol-4-yl)methyl)-1-(5-(trifluoromethyl)pyridin-2-yl)piperazine

(R)-2-methyl-4-((3-(1,2,3,6-tetrahydropyridin-4-y0-1H-pyrazol-4-yl)methyl)-1-(5-(trifluoromethyl)pyridin-2-yl)piperazine is synthesized in 5 steps as shown in scheme 5 above.

In Step 9-1, 10 ml of 2M Na₂CO₃, 10 ml of ethanol and 20 ml of toluene are added to a round-bottom flask containing tert-butyl 4-(4,4,5,5-tetramethyl-1,3,2-dioxaborolan-2-yl)-5,6-dihydropyridine-1(2H)-carboxylate (618 mg, 2 0 mmol), ethyl 3-iodo-1-(tetrahydro-2H-pyran-2-yl)-1H-pyrazole-4-carboxylate (350 mg, 1.0 mmol), Pd(PPh₃)₄ (116 mg, 0.1 mmol). The flask is purged with argon and sealed, the mixture is stirred at 80° C. for 18 hours, cooled to ambient temperature and then extracted with ethyl acetate. The organic layers are combined, dried and concentrated, and the residue is purified by silica gel column chromatography (elutated with EtOAc/Hexane) to give tert-butyl 4-(4-(ethoxycarbonyl)-1-(tetrahydro-2H-pyran-2-yl)-1H-pyrazol-3-yl)-5,6-dihydropyridine-1(2H)-carboxylate, m/z [M⁺+1] 406.2.

In step 9-2, LiBH₄ (190 mg, 8 7 mmol) is added to a solution tert-butyl 4-(4-(ethoxycarbonyl)-1-(tetrahydro-2H-pyran-2-yl)-1H-pyrazol-3-yl)-5,6-dihydropyridine-1(2H)-carboxylate (150 mg, 0.37 mmol) in THF (5 ml) with 0.1 ml of MeOH. The mixture is stirred at 70° C. for 24 hours until the ester disappears, the reaction is then quenched with 1N HCl and NaHCO₃ is used to adjust the pH to 5. The mixture is extracted with ethyl acetate (10 ml×3), and the organic layers are combined, dried and concentrated to give tert-butyl 4-(4-(hydroxymethyl)-1-(tetrahydro-2H-pyran-2-yl)-1H-pyrazol-3-yl)-5,6-dihydropyridine-1(2H)-carboxylate, m/z [M⁺+1] 364.2. The product is used without purification in step 9-3.

In step 9-3, activated MnO₂ (370 mg, 4.25 mmol) is added to tert-butyl 4-(4-(hydroxymethyl)-1-(tetrahydro-2H-pyran-2-yl)-1H-pyrazol-3-yl)-5,6-dihydropyridine-1(2H)-carboxylate (step 9-2) dissolved in 20 ml of CH₂Cl₂. The mixture is stirred for 3 hours in a 40° C. oil-bath and the MnO₂ is then removed by filtration. The filtrate is then concentrated to give tert-butyl 4-(4-formyl-1-(tetrahydro-2H-pyran-2-yl)-1H-pyrazol-3-yl)-5,6-dihydropyridine-1(2H)-carboxylate, m/z [M⁺+1] 362.2, which is used without purification in step 9-4

In step 9-4, (R)-2-methyl-1-(5-(trifluoromethyl)pyridin-2-yl)piperazine (0.105 g, 0.43 mmol), acetic acid (0.140 g, 2.3 mmol) and NaBH(OAc)₃ (0.25 g, 1.2 mmol) is added to tert-butyl 4-(4-formyl-1-(tetrahydro-2H-pyran-2-yl)-1H-pyrazol-3-yl)-5,6-dihydropyridine-1(2H)-carboxylate dissolved in 10 ml of CH₂Cl₂. The mixture is stirred for 18 hours at 40° C. until the reaction is completed, and the mixture is neutralized with addition of a 2M Na₂CO₃ solution. The mixture is then extracted with CH₂Cl₂ (20 ml×3), the organic layers are combined, dried, and concentrated, and the residue is purified with silica gel chromatography (eluted with hexane-ethyl acetate) to give tert-butyl 4-(4-(((R)-3-methyl-4-(5-(trifluoromethyl)pyridin-2-yl)piperazin-1-yl)methyl)-1-(tetrahydro-2H-pyran-2-yl)-1H-pyrazol-3-yl)-5,6-dihydropyridine-1(2H)-carboxylate, m/z [M⁺+1] 590.2.

In step 9-5, 2 ml of 5 M HCl in i-PrOH is added to a solution of tert-butyl 4-(4-(((R)-3-methyl-4-(5-(trifluoromethyl)pyridin-2-yl)piperazin-1-yl)methyl)-1-(tetrahydro-2H-pyran-2-yl)-1H-pyrazol-3-yl)-5,6-dihydropyridine-1(2H)-carboxylate (136 mg, 0.23 mmol) in 10 ml of methanol is added 2 ml of 5 M HCl in i-PrOH. The mixture is stirred at room temperature for 4 hours and the solvent is removed. The residue is re-dissolved in 10 ml CH₂Cl₂ containing 2 ml of TFA, is stirred for 10 minutes, concentrated and the residue is purified by HPLC purification (C₁₈ column, eluted with CH₃CN/H₂O with 0.035% TFA). The factions containing product are combined, neutralized with sodium carbonate, and extracted with ethyl acetate. The organic layers are then combined, dried, and concentrated to give (R)-2-methyl-4-((3-(1,2,3,6-tetrahydropyridin-4-yl)-1H-pyrazol-4-yl)methyl)-1-(5-(trifluoromethyl)pyridin-2-yl)piperazine. ¹H NMR (DMSO-d₆) δ 1.12 (d, 3H, J=6.8 Hz), 1.94 (t, 1H, J=8.8 Hz), 2.16 (d, 1H, J=10.8 Hz), 2.35 (m, 2H), 2.78 (d, 1H, J=11.2 Hz), 2.88 (m, 3H), 2.99 (m, 2H), 3.29 (m, 3H), 4.14 (d, 1H, J=12.8 Hz), 4.58 (b, 1H), 5.76 (s, 1H), 6.39 (s, 1H), 6.88 (d, 1H, J=8.8 Hz), 7.47 (bs, 1H), 7.79 (d, 1H, J=8.8 Hz), 8.40 (s, 1H); m/z [M⁺+1] 407.2.

### Example 10

### Preparation of (R)-4-(4-((3-methyl-4-(5-(trifluoromethyl)pyridin-2-yl)piperazin-1-yl)methyl)-1H-pyrazol-3-yl)-5,6-dihydropyridine-1(2H)-carbaldehyde

(R)-4-(4-((3-methyl-4-(5-(trifluoromethyl)pyridin-2-yl)piperazin-1-yl)methyl)-1H-pyrazol-3-yl)-5,6-dihydropyridine-1(2H)-carbaldehyde is synthesized using (R)-2-methyl-4-((3-(1,2,3,6-tetrahydropyridin-4-yl)-1H-pyrazol-4-yl)methyl)-1-(5-(trifluoromethyl)pyridin-2-yl)piperazine as shown in scheme 5.

To a solution of formic acid (3.4 mg, 0.074 mmol) and triethyl amine (15 mg, 0.148 mmol) in 1 ml of DMF is added HATU (28.1 mg, 0.074 mmol). The mixture is stirred for 10 minutes before a solution of (R)-2-methyl-4-((3-(1,2,3,6-tetrahydropyridin-4-yl)-1H-pyrazol-4-yl)methyl)-1-(5-(trifluoromethyl)pyridin-2-yl)piperazine (20 mg, 0.049 mmol) in 0.5 ml DMF is added. After 2 hours, the mixture is concentrated and the residue is purified by HPLC (C₁₈ column, eluted with CH₃CN/H₂O with 0.035% TFA). The factions containing product are combined, neutralized with sodium carbonate, and extracted with ethyl acetate. The organic layers are then combined, dried, and concentrated to give (R)-4-(4-((3-methyl-4-(5-(trifluoromethyl)pyridin-2-yl)piperazin-1-yl)methyl)-1H-pyrazol-3-yl)-5,6-dihydropyridine-1(2H)-carbaldehyde, ¹H NMR (DMSO-d₆) δ 1.13 (d, 3H, J=6.8 Hz), 1.94 (t, 1H, J=12 Hz), 2.17 (d, 1H, J=8.0 Hz), 2.56 (m, 1H), 2.63 (m, 1H), 2.81 (d, 1H, J=10.0 Hz), 2.90 (d, 1H, J=10.8 Hz), 2.99 (t, 1H, J=10.4 Hz), 3.29 (m, 2H), 3.59 (m, 2H), 4.11 (m, 3H), 4.59 (b, 1H), 6.51 (s, 1H), 6.88 (d, 1H, J=9.6 Hz), 7.63 (bs, 1H), 7.78 (d, 1H, J=9.6 Hz), 8.09 (s, 1H), 8.40 (s, 1H), 12.63 (bs, 1H); m/z [M⁺+1] 435.2.

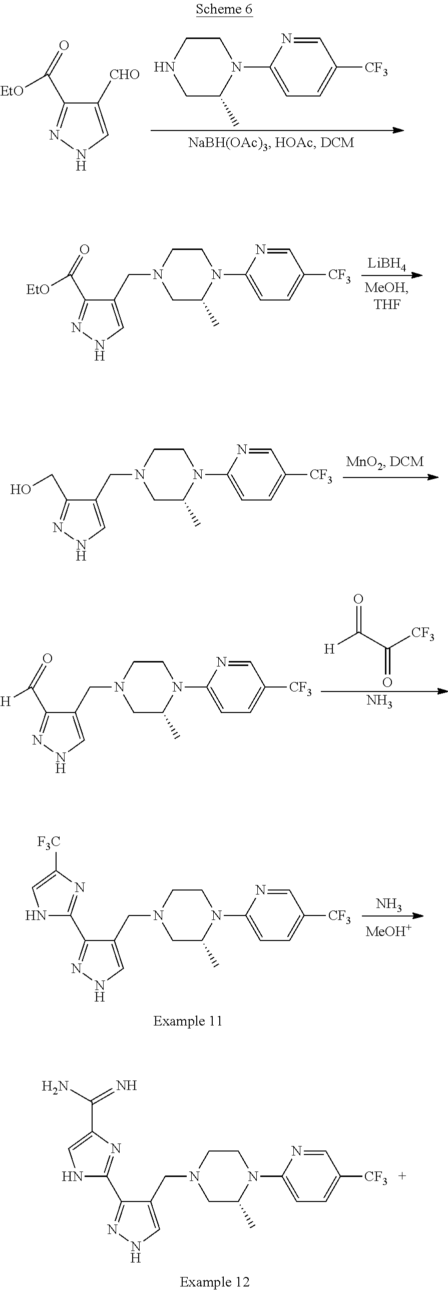

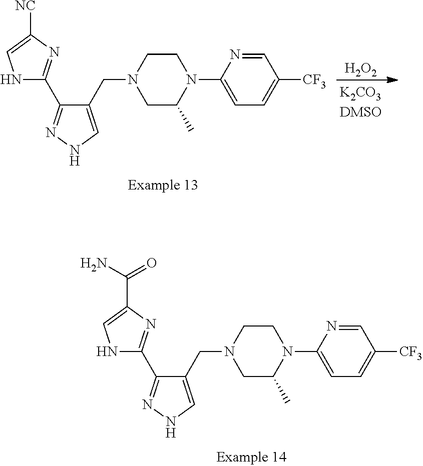

### Example 11

### Preparation of (R)-2-methyl-4-((3-(4-(trifluoromethyl)-1H-imidazol-2-yl)-1H-pyrazol-4-yl)methyl)-1-(5-(trifluoromethyl)pyridin-2-yl)piperazine

(R)-2-methyl-4-((3-(4-(trifluoromethyl)-1H-imidazol-2-yl)-1H-pyrazol-4-yl)methyl)-1-(5-(trifluoromethyl)pyridin-2-yl)piperazine is synthesized in 4 steps as shown in scheme 6 above.

In step 11-1, NaBH(OAc)₃ (0.85 g, 4.0 mmol) is added to a solution of ethyl 4-formyl-1H-pyrazole-3-carboxylate (0.336 g, 2.0 mmol), (R)-2-methyl-1-(5-(trifluoromethyl)pyridin-2-yl)piperazine (0.49 g, 2.0 mmol) and acetic acid (0.24 g, 4.0 mmol) in 20 ml of CH₂Cl₂. the mixture is stirred at room temperature for 18 hours until the reaction is completed, and saturated sodium carbonate solution is then added to adjust to pH 12. The mixture is extracted with CH₂Cl₂ (40 ml×3), and the organic layers are combined, dried, and concentrated to give crude (R)-ethyl 4-((3-methyl-4-(5-(trifluoromethyl)pyridin-2-yl)piperazin-1-yl)methyl)-1H-pyrazole-3-carboxylate. It is used without further purification, m/z [M⁺+1] 398.2.

In step 11-2, LiBH₄(0.20 g, 9 mmol) is added to (R)-ethyl 4-((3-methyl-4-(5-(trifluoromethyl)pyridin-2-yl)piperazin-1-yl)methyl)-1H-pyrazole-3-carboxylate (0.37 g, 0.93 mmol) dissolved in 20 ml of THF with 0.1 ml of methanol. The mixture is stirred at 70° C. for 24 hours until the ester disappears, the reaction is then quenched with 1N HCl and NaHCO₃ is used to adjust to pH 5. The mixture is extracted with CH₂Cl₂ (30 ml×3), and the organic layers are combined, dried and concentrated to give crude (R)-(4-((3-methyl-4-(5-(trifluoromethyl)pyridin-2-yl)piperazin-1-yl)methyl)-1H-pyrazol-3-yl)methanol, m/z ]M⁺+1] 356.2, which is used in step 11-3 without purification.

In step 11-3, activated MnO₂ (0.8 g, 9.2 mmol) is added to the crude compound (R)-(4-((3-methyl-4-(5-(trifluoromethyl)pyridin-2-yl)piperazin-1-yl)methyl)-1H-pyrazol-3-yl)methanol (0.328 g, 0.924 mmol) (from step 11-2) dissolved in 30 ml of CH₂Cl₂. The mixture is stirred for 2 hours in a 40° C. oil-bath until the reaction is complete, and the MnO₂ is then removed by filtration. The filtrate is concentrated, and purified by silica gel chromatography (eluted with hexane-ethyl acetate) to give (R)-4-((3-methyl-4-(5-(trifluoromethyl)pyridin-2-yl)piperazin-1-yl)methyl)-1H-pyrazole-3-carbaldehyde, m/z [M⁺+1] 354.2.

In step 11-4, 1,1,1-trifluoro-3,3-dibromoacetone (42 mg, 0.155 mmol) is added to a solution of sodium acetate trihydrate (42.3 mg, 0.31 mmol) in water. The mixture is stirred under reflux for 30 minutes in 115° C. oil bath to form 3,3,3-trifluoro-2-oxopropanal in-situ. After cooling to room temperature, the solution is added to a methanol (3 ml) solution containing (R)-4-((3-methyl-4-(5-(trifluoromethyl)pyridin-2-yl)piperazin-1-yl)methyl)-1H-pyrazole-3-carbaldehyde (50 mg, 0.141 mmol) and 0.5 ml of concentrated ammonium hydroxide. The mixture is stirred at room temperature for 16 hours and then concentrated and the residue is purified by HPLC (C₁₈ column, eluted with CH₃CN/H₂O with 0.035% TFA). The factions containing product are combined, neutralized with sodium carbonate, and extracted with ethyl acetate. The organic layers are then combined, dried, and concentrated to give (R)-2-methyl-4-((3-(4-(trifluoromethyl)-1H-imidazol-2-yl)-1H-pyrazol-4-yl)methyl)-1-(5-(trifluoromethyl)pyridin-2-yl)piperazine, ¹H NMR (DMSO-d₆) δ 1.14 (d, 3H, J=6.8 Hz), 2.06 (m, 1H), 2.22 (m, 1H), 2.86 (d, 1H, J=10.8 Hz), 2.99 (d, 1H, J=10.8 Hz), 3.09 (t, 1H, J=12 Hz), 3.72 (d, 1H, J=13.6 Hz), 3.80 (d, 1H, J=13.6 Hz), 4.16 (d, 1H, J=12.0 Hz), 4.58 (b, 1H), 6.88 (d, 1H, J=8.8 Hz), 7.76 (s, 1H), 7.78 (d, 1H, J=8.8 Hz), 7.79 (s, 1H), 8.40 (s, 1H); m/z [M⁺+1] 460.2.

### Examples 12 and 13

### Preparation of (R)-2-(4-((3-methyl-4-(5-(trifluoromethyl)pyridin-2-yl)piperazin-1-yl)methyl)-1H-pyrazol-3-yl)-1H-imidazole-4-carboximidamide (Example 12) and

### (R)-2-(4-((3-methyl-4-(5-(trifluoromethyl)pyridin-2-yl)piperazin-1-yl)methyl)-1H-pyrazol-3-yl)-1H-imidazole-4-carbonitrile (Example 13)

(R)-2-(4-((3-methyl-4-(5-(trifluoromethyl)pyridin-2-yl)piperazin-1-yl)methyl)-1H-pyrazol-3-yl)-1H-imidazole-4-carboximidamide (Example 12) and (R)-2-(4-((3-methyl-4-(5-(trifluoromethyl)pyridin-2-yl)piperazin-1-yl)methyl)-1H-pyrazol-3-yl)-1H-imidazole-4-carbonitrile (Example 13) are synthesized as shown in scheme 6.

To a flask containing (R)-2-methyl-4-((3-(4-(trifluoromethyl)-1H-imidazol-2-yl)-1H-pyrazol-4-yl)methyl)-1-(5-(trifluoromethyl)pyridin-2-yl)piperazine (65 mg, 0.14 mmol) is added 10 ml of 5% ammonium hydroxide solution and 2 ml of MeOH. The mixture is heated at 60° C. for 18 hours and then concentrated. The residue is purified by HPLC (C₁₈ column, eluted with CH₃CN/H₂O with 0.035% TFA). Both (R)-2-(4-((3-methyl-4-(5-(trifluoromethyl)pyridin-2-yl)piperazin-1-yl)methyl)-1H-pyrazol-3-yl)-1H-imidazole-4-carboximidamide and (R)-2-(4-((3-methyl-4-(5-(trifluoromethyl)pyridin-2-yl)piperazin-1-yl)methyl)-1H-pyrazol-3-yl)-1H-imidazole-4-carbonitrile are isolated. (R)-2-(4-((3-methyl-4-(5-(trifluoromethyl)pyridin-2-yl)piperazin-1-yl)methyl)-1H-pyrazol-3-yl)-1H-imidazole-4-carboximidamide (Example 12): ¹H NMR (DMSO-d₆) δ 1.14 (d, 3H, J=6.4 Hz), 2.06 (m, 1H), 2.26 (m, 1H), 2.88(d, 1H, J=11.6 Hz), 2.96 (d, 1H, J=11.6 Hz), 3.06 (t, 1H, J=12 Hz), 3.76 (d, 1H, J=14 Hz), 3.88 (d, 1H, J=14 Hz), 4.15 (d, 1H, J=12.4 Hz), 4.59 (b, 1H), 6.89 (d, 1H, J=8.8 Hz), 7.77 (dd, 1H, J₁=8.8 Hz, J₂=2.0 Hz), 7.82 (s, 1H), 8.24 (s, 1H), 8.39 (s, 1H), 8.57 (b, 2H), 8.83 (b, 2H), 13.21 (bs, 1H); m/z [M⁺+1] 434.2. (R)-2-(4-((3-methyl-4-(5-(trifluoromethyl)pyridin-2-yl)piperazin-1-yl)methyl)-1H-pyrazol-3-yl)-1H-imidazole-4-carbonitrile (Example 13): m/z [M⁺+1] 417.2)

### Example 14

### Preparation of (R)-2-(4-((3-methyl-4-(5-(trifluoromethyl)pyridin-2-yl)piperazin-1-yl)methyl)-1H-pyrazol-3-yl)-1H-imidazole-4-carboxamide

(R)-2-(4-((3-methyl-4-(5-(trifluoromethyl)pyridin-2-yl)piperazin-1-yl)methyl)-1H-pyrazol-3-yl)-1H-imidazole-4-carboxamide is synthesized using (R)-2-(4-((3-methyl-4-(5-(trifluoromethyl)pyridin-2-yl)piperazin-1-yl)methyl)-1H-pyrazol-3-yl)-1H-imidazole-4-carbonitrile as shown in scheme 6 using the method described above for Example 2.

To a solution of (R)-2-(4-((3-methyl-4-(5-(trifluoromethyl)pyridin-2-yl)piperazin-1-yl)methyl)-1H-pyrazol-3-yl)-1H-imidazole-4-carbonitrile (38 mg, 0.091 mmol) in 2 ml of DMSO is added K₂CO₃ (60 mg, 0.43 mmol) and 30% H₂O₂ (0.30 ml). The reaction is heated at 40° C. for 72 hours, then cooled to room temperature. It is purified by HPLC (C₁₈ column, eluted with CH₃CN/H₂O with 0.035% TFA). The factions containing product are combined, neutralized with sodium carbonate, and extracted with ethyl acetate. The organic layers are combined, dried, and concentrated to give (R)-2-(4-((3-methyl-4-(5-(trifluoromethyl)pyridin-2-yl)piperazin-1-yl)methyl)-1H-pyrazol-3-yl)-1H-imidazole-4-carboxamide, m/z [M⁺+1] 435.2.

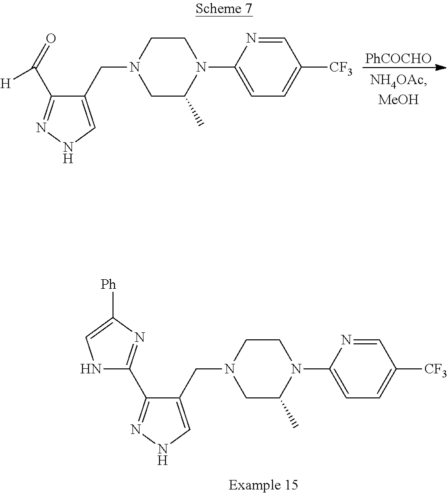

### Example 15

### Preparation of (R)-2-methyl-4-((3-(4-phenyl-1H-imidazol-2-yl)-1H-pyrazol-4--yl)methyl)-1-(5-(trifluoromethyl)pyridin-2-yl)piperazine

(R)-2-methyl-4-((3-(4-phenyl-1H-imidazol-2-yl)-1H-pyrazol-4-yl)methyl)-1-(5-(trifluoromethyl)pyridin-2-yl)piperazine is synthesized as shown in scheme 7.

To a solution of phenylglyoxal (4 mg, 0.028 mmol) and (R)-4-((3-methyl-4-(5-(trifluoromethyl)pyridin-2-yl)piperazin-1-yl)methyl)-1H-pyrazole-3-carbaldehyde (10 mg, 0.028 mmol) in 2 ml of methanol is added ammonium acetate (22 mg, 0.28 mmol). The mixture is stirred at room temperature for 1 hour and then concentrated. The residue is purified by HPLC (C₁₈ column, eluted with CH₃CN/H₂O with 0.035% TFA). The factions containing the title compound are combined, neutralized with sodium carbonate, and extracted with ethyl acetate. The organic layers are then combined, dried, and concentrated to give (R)-2-methyl-4-((3-(4-phenyl-1H-imidazol-2-yl)-1H-pyrazol-4-yl)methyl)-1-(5-(trifluoromethyl)pyridin-2-yl)piperazine, m/z [M⁺+1] 468.2.

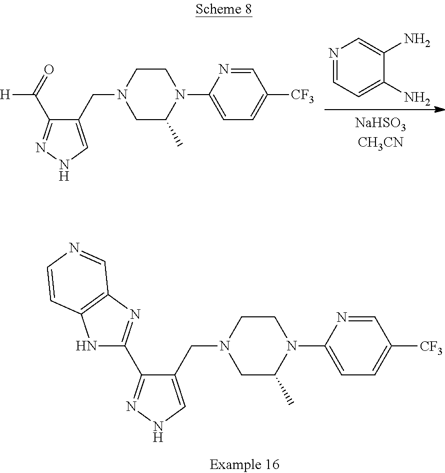

### Example 16

### Preparation of (R)-2-(4-((3-methyl-4-(5-(trifluoromethyl)pyridin-2-yl)piperazin-1-yl)methyl)-1H-pyrazol-3-yl)-1H-imidazol4,5-c]pyridine

(R)-2-(4-((3-methyl-4-(5-(trifluoromethyl)pyridin-2-yl)piperazin-1-yl)methyl)-1H-pyrazol-3-yl)-1H-imidazol4,5-c]pyridine is synthesized as shown in scheme 8.

To a solution of (R)-4-((3-methyl-4-(5-(trifluoromethyl)pyridin-2-yl)piperazin-1-yl)methyl)-1H-pyrazole-3-carbaldehyde (20 mg, 0.056 mmol) and pyridine-3,4-diamine (8 mg, 0.073 mmol) in 1 ml of acetonitrile in a microwave tube is added NaHSO₃ (8.9 mg, 0.085 mmol). The tube is sealed and the mixture is heated at 160° C. in microwave for 15 minutes until the reaction is complete. After cooling to room temperature, water is added and the mixture is then extracted with ethyl acetate (5 ml×3). The organic layers are combined, dried and concentrated, and the residue is purified by HPLC (C₁₈ column, eluted with CH₃CN/H₂O with 0.035% TFA). The factions containing the title compound are combined, neutralized with sodium carbonate, and extracted with ethyl acetate. The organic layers are then combined, dried, and concentrated to give (R)-2-(4-((3-methyl-4-(5-(trifluoromethyl)pyridin-2-yl)piperazin-1-yl)methyl)-1H-pyrazol-3-yl)-1H-imidazo[4,5-c]pyridine, ¹H NMR (DMSO-d₆) δ 1.20 (d, 3H, J=6.4 Hz), 2.14 (m, 1H), 2.31 (m, 1H), 2.93(d, 1H, J=10.8 Hz), 3.06 (d, 1H, J=11.2 Hz), 3.13 (t, 1H, J=12.4 Hz), 3.95 (d, 1H, J=14 Hz), 4.00 (d, 1H, J=14 Hz), 4.18 (d, 1H, J=12.4 Hz), 4.60 (b, 1H), 6.88 (d, 1H, J=9.6 Hz), 7.51 (b, 1H), 7.65 (b, 1H), 7.77 (d, 1H, J=8.0 Hz), 7.88 (s, 1H), 8.28 (d, 1H, J=9.6 Hz), 8.40 (s, 1H), 8.90 (bs, 1H); m/z [M⁺+1] 443.2.

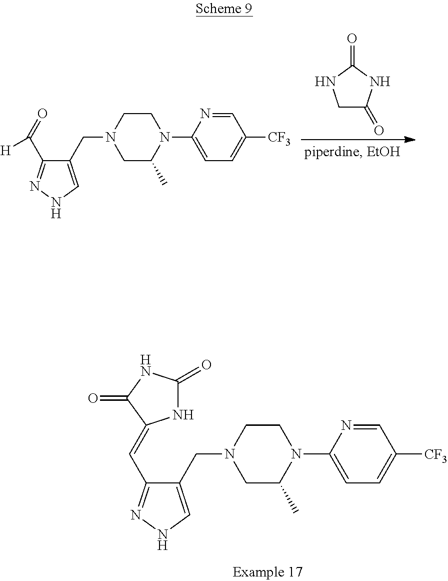

### Example 17

### Preparation of R-5-((4-((3-methyl-4-(5-(trifluoromethyl)pyridin-2-yl)piperazin-1-yl)methyl)-1H-pyrazol-3-yl)methylene)imidazolidine-2,4-dione

R-5-((4-((3-methyl-4-(5-(trifluoromethyl)pyridin-2-yl)piperazin-1-yl)methyl)-1H-pyrazol-3-yl)methylene)imidazolidine-2,4-dione is synthesized as shown in scheme 9.

To a solution of (R)-4-((3-methyl-4-(5-(trifluoromethyl)pyridin-2-yl)piperazin-1-yl)methyl)-1H-pyrazole-3-carbaldehyde (20 mg, 0.056 mmol) and hydantoin (imidazolidine-2,4-dione) (71 mg, 0.71 mmol) in 5 ml of ethanol is added piperidine (10 μl). The flask is sealed and heated to 115° C. in an oil-bath for 16 hours and then cooled to room temperature, concentrated and purified by HPLC (C₁₈ column, eluted with CH₃CN/H₂O with 0.035% TFA). The fractions containing the title compound are combined and lyophilized to give the TFA salt of R-5-((4-((3-methyl-4-(5-(trifluoromethyl)pyridin-2-yl)piperazin-1-yl)methyl)-1H-pyrazol-3-yl)methylene)imidazolidine-2,4-dione, ¹H NMR (DMSO-d₆) δ 1.22 (d, 3H, J=7.2 Hz), 3.07 (m, 1H), 3.25 (m, 2H), 3.44 (m, 2H), 4.44 (m, 2H), 4.52 (d, 1H, J=13 Hz), 4.94 (b, 1H), 6.70 (s, 1H), 7.03 (d, 1H, J=8.8 Hz), 7.91 (d, 1H, J=8.8 Hz), 8.00 (s, 1H), 8.48 (s, 1H), 9.52 (bs, 1H), 9.61 (bs, 1H), 11.32 (s, 1H); m/z [M⁺+1] 436.2.

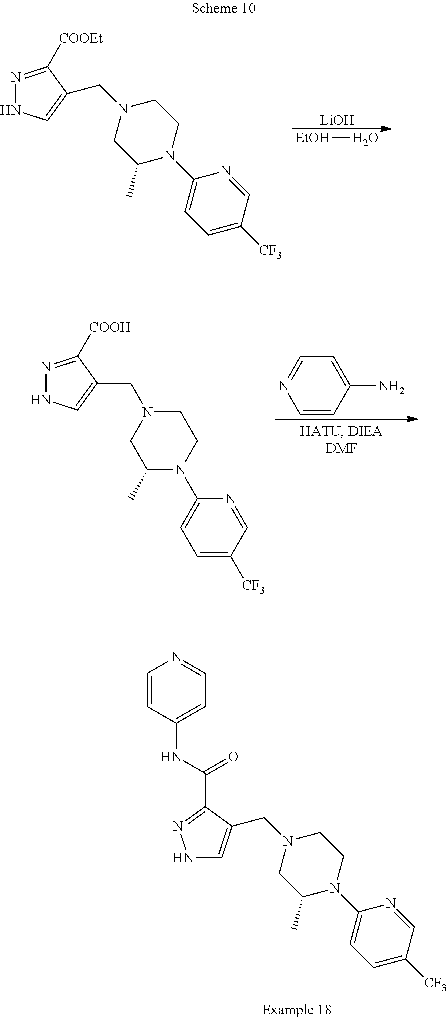

### Example 18

### Preparation of (R)-4-((3-methyl-4-(5-(trifluoromethyl)pyridin-2-yl)piperazin-1-yl)methyl)-N-(pyridin-4-yl)-1H-pyrazole-3-carboxamide

(R)-4-((3-methyl-4-(5-(trifluoromethyl)pyridin-2-yl)piperazin-1-yl)methyl)-N-(pyridin-4-yl)-1H-pyrazole-3-carboxamide is synthesized in 2 steps as shown in scheme 10.

In step 18-1, LiOH (0.20 g, 4.7 mmol) is added to a solution of (R)-ethyl 4-((3-methyl-4-(5-(trifluoromethyl)pyridin-2-yl)piperazin-1-yl)methyl)-1H-pyrazole-3-carboxylate (0.467 g, 1.17 mmol) in 20 ml of EtOH-H₂O (1:1). The solution is stirred at room temperature for 48 hours until the reaction is completed, 1N HCl is then added to adjust to pH 4. The mixtures is lyophilized to give (R)-4-((3-methyl-4-(5-(trifluoromethyl)pyridin-2-yl)piperazin-1-yl)methyl)-1H-pyrazole-3-carboxylic acid along with LiCl. The acid is used without further purification.

In step 18-2: HATU (29 mg, 0.076 mmol) is added to a solution of (R)-4-((3-methyl-4-(5-(trifluoromethyl)pyridin-2-yl)piperazin-1-yl)methyl)-1H-pyrazole-3-carboxylic acid (20 mg, 0.054 mmol), 4-aminopyridine (15.3 mg, 0.16 mmol) and ethyl-N,N-diisopropylamine (21 mg, 0.16 mmol) in DMF. The mixture is stirred for 16 hours and then concentrated. The residue is purified by HPLC (C₁₈ column, eluted with CH₃CN/H₂O with 0.035% TFA). The factions containing compound (R)-4-((3-methyl-4-(5-(trifluoromethyl)pyridin-2-yl)piperazin-1-yl)methyl)-N-(pyridin-4-yl)-1H-pyrazole-3-carboxamide are combined, neutralized with sodium carbonate, and extracted with ethyl acetate. The organic layers are then combined, dried, and concentrated to give (R)-4-((3-methyl-4-(5-(trifluoromethyl)pyridin-2-yl)piperazin-1-yl)methyl)-N-(pyridin-4-yl)-1H-pyrazole-3-carboxamide, ¹H NMR (DMSO-d₆) δ 1.13 (d, 3H, J=6.8 Hz), 2.08 (m, 1H), 2.18 (m, 1H), 2.79 (d, 1H, J=11.2 Hz), 3.02 (d, 1H, J=10.8 Hz), 3.11 (t, 1H, J=12 Hz), 3.70 (d, 1H, J=13.6 Hz), 3.81 (d, 1H, J=13.6 Hz), 4.19 (d, 1H, J=12.0 Hz), 4.57 (b, 1H), 6.88 (d, 1H, J=9.6 Hz), 7.77 (d, 1H, J=9.6 Hz), 7.79 (d, 2H, J=6.4 Hz), 7.84 (s, 1H), 8.40 (s, 1H), 8.44 (d, 2H, J=6.4 Hz), 10.69 (s, 1H); m/z [M⁺+1] 446.2.

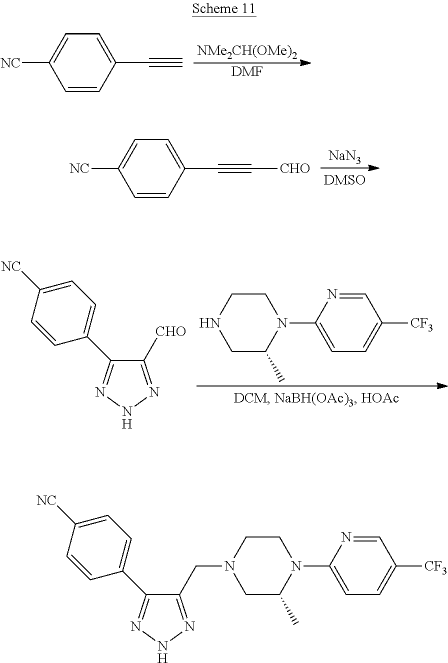

### Example 19

### (R)-4-(5-((3-methyl-4-(4-(trifluoromethyl)phenyl)piperazin-1-yl)methyl)-2H-1,2,3-triazol-4-yl)benzonitrile

(R)-4-(5-((3-methyl-4-(4-(trifluoromethyl)phenyl)piperazin-1-yl)methyl)-2H-1,2,3-triazol-4-yl)benzonitrile is synthesized in 3 steps as shown in scheme 11.

In step 19-1, a mixture of 4-ethynylbenzonitrile (0.508 g, 4 mmol) and N,N-dimethylformamide dimethylacetal (0.952 g, 8 mmol) in DMF (1 ml) is heated to 70° C. for 72 hours. The mixture is cooled to room temperature and poured into cold 1N HCl and extracted with ethyl acetate. The organic layers are combined, dried and concentrated and the residue is purified by silica gel column chromatography (elutated with EtOAc/Hexane) to give 4-(3-oxoprop-1-ynyl)benzonitrile. ¹H NMR (DMSO-d₆) δ 7.89 (d, 2H, J=8.0 Hz), 7.99 (d, 2H, J=8.0 Hz), 9.47 (s, 1H); m/z [M⁺+1] 156.1.

In step 19-2 a vigorously stirred solution of NaN₃ (71.5 mg, 1 1 mmol) in DMSO (3 ml) is kept at 20° C. in a water bath. To this solution is added a solution of 4-(3-oxoprop-1-ynyl)benzonitrile (155 mg, 1.0 mmol) in DMSO (1 ml) over 10 minutes. The reaction is stirred for another 30 minutes at 20° C. and poured to a 15% aqueous KH₂PO₄ solution. The resulting precipitate formed is collected by vacuum filtration, and is washed with water and air-dried to give 4-(5-formyl-2H-1,2,3-triazol-4-yl)benzonitrile for use, without purification, in step 19-3.

In step 19-3, NaBH(OAc)₃ (63.6 mg, 0.3 mmol) is added to a solution of 4-(5-formyl-2H-1,2,3-triazol-4-yl)benzonitrile from step 2 (20 mg, 0 1 mmol), (R)-2-methyl-1-(5-(trifluoromethyl)pyridin-2-yl)piperazine (37 mg, 0.15 mmol) and acetic acid (24 mg, 0 4 mmol) in 3 ml of CH₂Cl₂. The mixture is stirred at room temperature for 15 minutes or until the reaction is completed. The mixture is concentrated and the residue is purified with HPLC (C₁₈ column, eluted with CH₃CN/H₂O with 0.035% TFA). The factions containing product are combined, neutralized with sodium carbonate, and extracted with ethyl acetate. The organic layers are combined, dried, and concentrated to give (R)-4-(5-((3-methyl-4-(4-(trifluoromethyl)phenyl)piperazin-1-yl)methyl)-2H-1,2,3-triazol-4-yl)benzonitrile. ¹H NMR (DMSO-d₆) δ 1.06 (d, 3H, J=6.4 Hz), 2.08 (m, 1H), 2.30 (dd, 1H, J₁=3.2 Hz, J₂=11.6 Hz), 2.77 (d, 1H, J=11.2 Hz), 2.89 (d, 1H, J=10.8 Hz), 2.99 (m, 1H), 3.71 (m, 2H), 4.16 (d, 1H, J=13.2 Hz), 4.60 (b, 1H), 6.88 (d, 1H, J=7.2 Hz), 7.78 (dd, 1H, J₁=7.2 Hz, J₂=2.8 Hz), 7.91 (d, 2H, J=8.4 Hz), 8.20 (d, 2H, J=8.4 Hz), 8.40 (d, 1H, J=2.8 Hz); m/z [M⁺+1] 427.2.

Other representative compounds of Formulas (I)-(XIX), prepared following the procedures described above, are set forth in Table 1.

TABLE 1

| | | Physical Data | |
|---|---|---|---|
| Example | Structure | MS (m/z): (M + 1) | IC₅₀ (nM) |
| 20 | 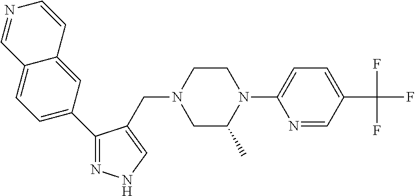 | 453.2 | 11 |
| 21 | 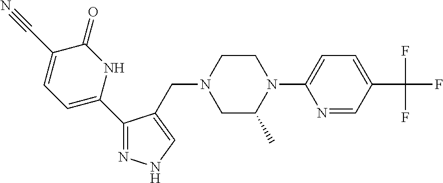 | 444.2 | 49 |
| 22 | 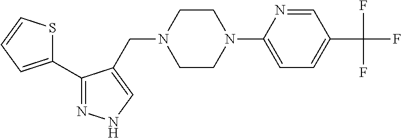 | 394.1 | 4325 |
| 23 | 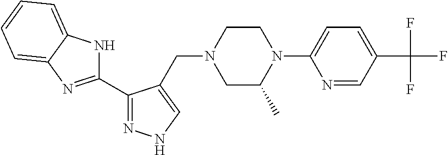 | 444.2 | 374 |
| 24 | 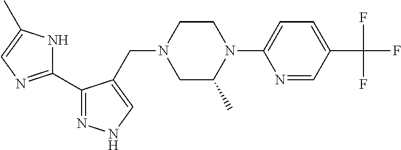 | 406.2 | 10000 |
| 25 | 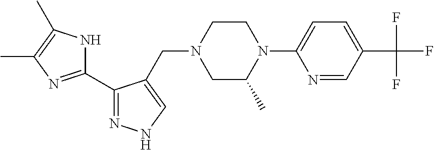 | 420.2 | >10000 |
| 26 | 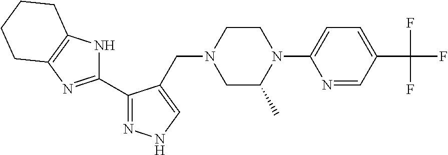 | 446.2 | 5012 |
| 27 | 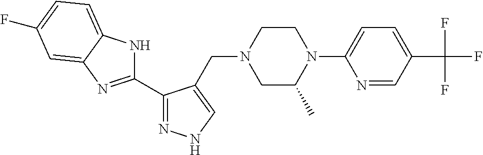 | 460.2 | 9798 |
| 28 | 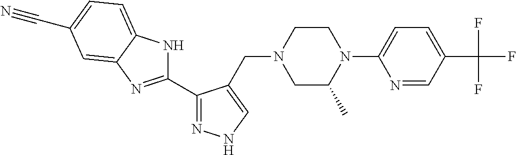 | 467.2 | 2448 |
| 29 | 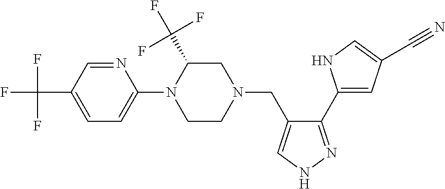 | 470.1 | 9 |
| 30 | 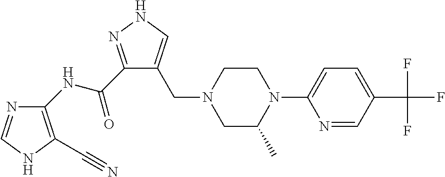 | 460.2 | >10000 |
| 31 | 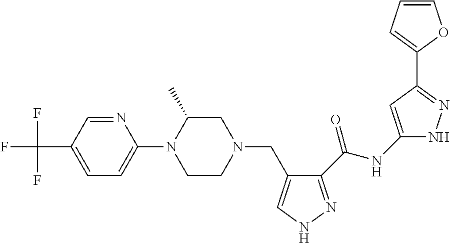 | 501.2 | 5332 |
| 32 | 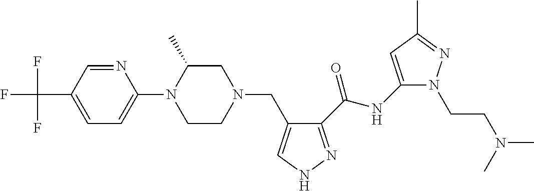 | 520.3 | 6194 |
| 33 | 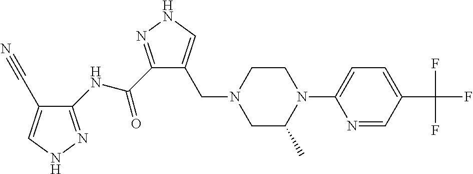 | 460.2 | >10000 |
| 34 | 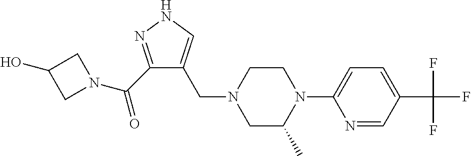 | 425.2 | >10000 |
| 35 | 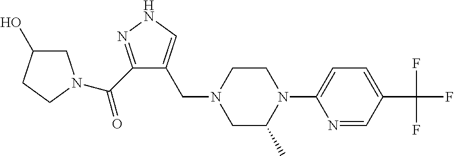 | 439.2 | >10000 |
| 36 | 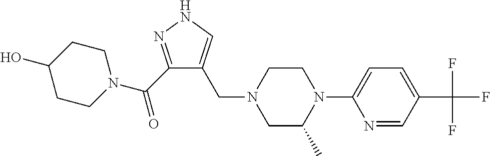 | 453.2 | >10000 |
| 37 | 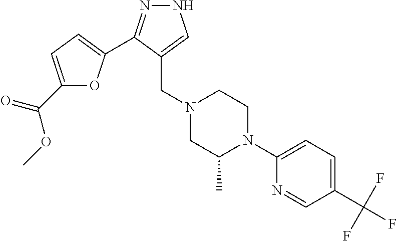 | 450.2 | 984 |
| 38 | 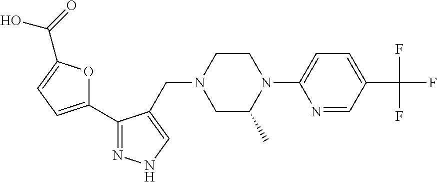 | 436.2 | 435 |
| 39 | 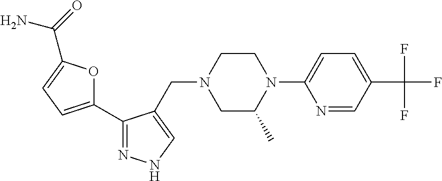 | 435.2 | 8 |
| 40 | 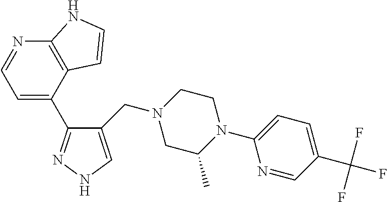 | 442.2 | 478 |
| 41 | 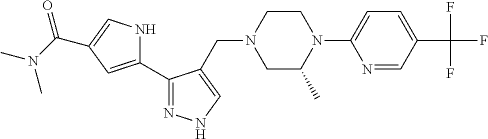 | 462.2 | 85 |
| 42 | 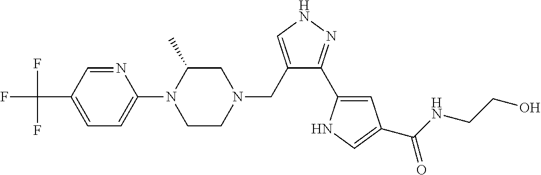 | 478.2 | 14 |
| 43 | 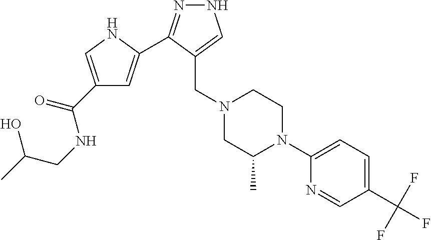 | 492.2 | 14 |
| 44 | 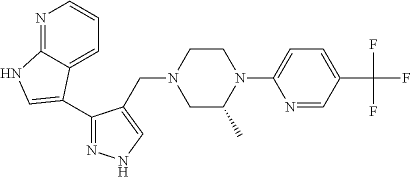 | 442.2 | 182 |
| 45 | 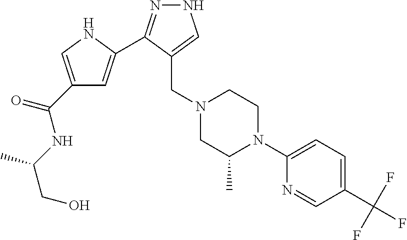 | 492.2 | 6 |
| 46 | 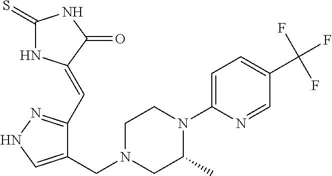 | 452.1 | >10000 |
| 47 | 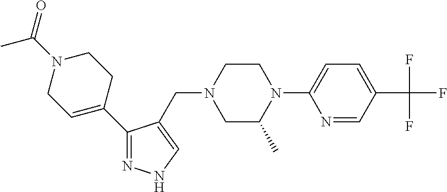 | 449.2 | 66 |
| 48 | 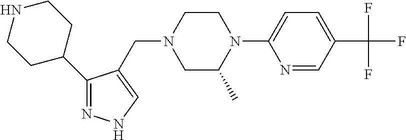 | 409.2 | 431 |
| 49 | 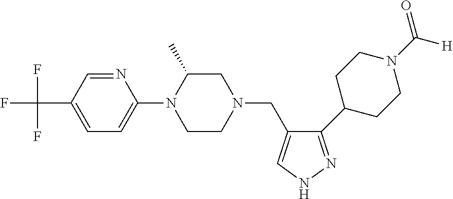 | 437.2 | 241 |
| 50 | 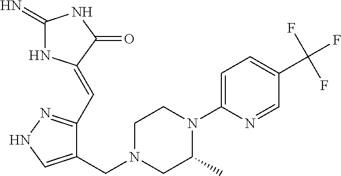 | 435.2 | 99 |
| 51 | 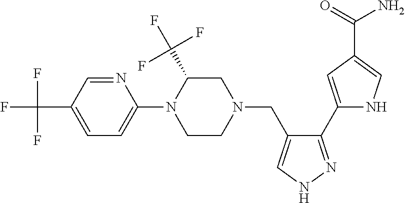 | 488.2 | 2 |
| 52 | 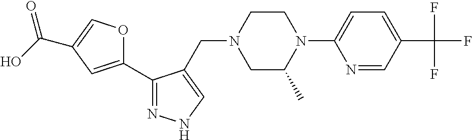 | 436.2 | 1042 |
| 53 | 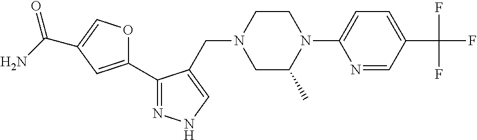 | 435.2 | 78 |
| 54 | 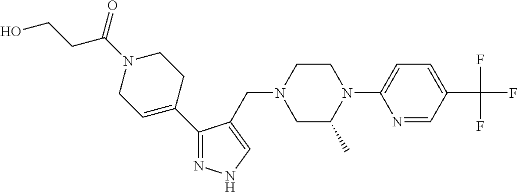 | 479.2 | 80 |

### Assays

Compounds of the present invention are assayed to measure their capacity to inhibit ITPKb according to the following assays.

### Purification of ITPKb

The DNA sequence encoding murine ITPKb residues 640-942 is amplified from a full-length construct in mammalian expression vector pKDNZ by PCR. The 3′-primer incorporates a stop codon and an overhanging PacI site. The product is digested with Pad before being ligated into the MH4 plasmid which has been prepared by digestion with PmlI and PacI. Cloning into the MH4 plasmid adds the sequence MGSDKIHHHHHH to the N-terminus of the translated region. Mutant enzymes are made by site-directed mutagenesis using the Stratagene Quikchange kit.

ITPKb is expressed in the HK100 strain of Escherichia coli. Typically, a 4 L batch of cells is grown in LB with 0.1 μg/mL ampicillin to 0.5 A₆₀₀ at 30 degrees C., before induction with 0.02% L-arabinose for 6 hours. Cells are harvested by centrifugation, and pellets are resuspended in 50 mL of 50 mM Tris (pH 8), 100 mM NaCl, 1 mM TCEP, and 0.1 mg/mL lysozyme, with 1 Complete protease inhibitor tablet (Roche). Cells are disrupted by sonication, and debris is removed by centrifugation for 40 minutes at 35000 g.

Initial purification is performed using three nickel-Sepharose Hi-Trap HP 1 mL columns (Amersham) connected in series. After application of the pellet supernatants, the bound material is washed with 20 mM Tris (pH 8.0), 20 mM imidazole, 10% glycerol (v/v), and 1 mM TCEP before elution with an imidazole gradient up to 200 mM.

Fractions containing ITPKb are identified by SDS-PAGE, and the pure fractions are concentrated and buffer exchanged using centriprep 20 15 kDa columns into 20 mM Tris (pH 8), 200 mM KCl, 5 mM MgCl₂, 0.5 mM DTT, 10% glycerol, 1 μM IP₃, and 20 μM ATP to a final protein concentration of 7 mg/mL.

### Biochemical Measurement of ITPKb Activity

ITPKb activity is determined using the Kinase-Glo (Promega) ATP depletion assay. The assay reaction buffer consists of 50 mM Tris (pH 8.0), 100 mM NaCl, 1 mM DTT, 10% glycerol, 5 mM MgCl₂, 1 μM ATP, and 10 μM IP₃ (Alexis Biochemicals). 50 nl of inhibitor is then added to each 40 μL reaction followed by a 10 μL addition of purified ITPKb (final concentration of 60 nM). The reaction mixture is incubated for 60 minutes at room temperature and stopped by the addition of an equal volume of kinase-glo reagent (Promega). Luminescence is measured using a Molecular Devices Acquest instrument.

### Measuring Intracellular IP3, IP4, and IP5 Levels by HPLC

Jurkat cells are obtained from ATCC (clone E6-1) (ATCC Cat#TIB-152). 10⁷ cells in 1 ml of inositol free RPMI-1640 w/o serum, are pulse labeled at 37° C. for 6 hours with 15 uCi of 3H myo-inositol in inositol. Cells are then diluted to 4 ml of RPMI-1640 with 10% FBS and incubated overnight at 37° C. Cells are then concentrated and resuspended in 1 ml of RPMI-1640 w/10% FBS. 1 μl of inhibitor in DMSO is then added. 50 μg of OKT3 and 10 μg of anti-human CD28 (BD Pharmingen clone CD28.2) is added followed by a 5 minute incubation at 37° C. Cells are then concentrated and the reaction quenched with the resuspension of the cell pellet in 100 μL of PBS w/350 mM HCl. Extracts are then spun to remove proteins and cellular debris. Labeled inositol polyphosphates in the extracts are then resolved by HPLC on a Partisphere SAX column (15 cm×4.6 mm) Samples are eluted as follows with gradients generated by mixing buffer A (10 mM (NH₄)H₂PO₄, pH 3.35, with H₃PO₄) with buffer B (1.7 M (NH₄)H₂PO₄, pH 3.35, with H₃PO₄). 0-12.5 minutes 0-100% Buffer B; 12-5-25 minutes 100% Buffer B; 25-30 minutes 0-100% buffer A; 30-45 minutes 100% buffer A. Radioactivity is detected with an online β-Ram detector from IN/US systems.

Compounds of Formula I have an IC₅₀ in the range of 0.5 nM to 20 μM for inhibiting the phosphorylation of IP3 to IP4, while other compounds of Formula I have an in the range of 0.5 nM to 10 μM for inhibiting the phosphorylation of IP3 to IP4. Other compounds of Formula I have an IC₅₀ in the range of 0.5 nM to 8 μM for inhibiting the phosphorylation of IP3 to IP4, while other compounds of Formula I have an in the range of 0.5 nM to 6 μM for inhibiting the phosphorylation of IP3 to IP4. Other compounds of Formula I have an IC₅₀ in the range of 0.5 nM to 5 μM for inhibiting the phosphorylation of IP3 to IP4, while other compounds of Formula I have an IC₅₀ in the range of 0.5 nM to 2.5 μM for inhibiting the phosphorylation of IP3 to IP4. Other compounds of Formula I have an IC₅₀ in the range of 0.5 nM to 2 μM for inhibiting the phosphorylation of IP3 to IP4, while other compounds of Formula I have an IC₅₀ in the range of 0.5 nM to 1.5 μM for inhibiting the phosphorylation of IP3 to IP4. Other compounds of Formula I have an IC₅₀ in the range of 0.5 nM to 1 μM for inhibiting the phosphorylation of IP3 to IP4, while other compounds of Formula I have an IC₅₀ in the range of 0.5 nM to 800 nM for inhibiting the phosphorylation of IP3 to IP4. Other compounds of Formula I have an IC₅₀ in the range of 0.5 nM to 600 nM for inhibiting the phosphorylation of IP3 to IP4, while other compounds of Formula I have an IC₅₀ in the range of 0.5 nM to 500 nM for inhibiting the phosphorylation of IP3 to IP4. Other compounds of Formula I have an IC₅₀ in the range of 0.5 nM to 400 nM for inhibiting the phosphorylation of IP3 to IP4, while other compounds of Formula I have an IC₅₀ in the range of 0.5 nM to 300 nM for inhibiting the phosphorylation of IP3 to IP4. Other compounds of Formula I have an IC₅₀ in the range of 0.5 nM to 200 nM for inhibiting the phosphorylation of IP3 to IP4, while other compounds of Formula I have an IC₅₀ in the range of 0.5 nM to 100 nM for inhibiting the phosphorylation of IP3 to IP4. Other compounds of Formula I have an IC₅₀ in the range of 0.5 nM to 50 nM for inhibiting the phosphorylation of IP3 to IP4, while other compounds of Formula I have an IC₅₀ in the range of 0.5 nM to 20 nM for inhibiting the phosphorylation of IP3 to IP4. Other compounds of Formula I have an IC₅₀ in the range of 0.5 nM to 10 nM for inhibiting the phosphorylation of IP3 to IP4, while other compounds of Formula I have an IC₅₀ in the range of 0.5 nM to 5 nM for inhibiting the phosphorylation of IP3 to IP4.

Certain compounds of Formula I provided herein have an IC₅₀ of less than 10 μM in inhibiting the conversion of IP3 to IP4, while other compounds of Formula I provided herein have an IC₅₀ of less than 5 μM in inhibiting the conversion of IP3 to IP4. Certain compounds of Formula I provided herein have an IC₅₀ of less than 1 μM in inhibiting the conversion of IP3 to IP4, while other compounds of Formula I provided herein have an IC₅₀ of less than 500 nM in inhibiting the conversion of IP3 to IP4. Certain compounds of Formula I provided herein have an IC₅₀ of less than 250 nM in inhibiting the conversion of IP3 to IP4. Certain compounds of Formula I provided herein have an IC₅₀ of less than 200 nM in inhibiting the conversion of IP3 to IP4. Certain compounds of Formula I provided herein have an IC₅₀ of less than 150 nM in inhibiting the conversion of IP3 to IP4. Certain compounds of Formula I provided herein have an IC₅₀ of less than 100 nM in inhibiting the conversion of IP3 to IP4. Certain compounds of Formula I provided herein have an IC₅₀ of less than 50 nM in inhibiting the conversion of IP3 to IP4. Certain compounds of Formula I provided herein have an IC₅₀ of less than 25 nM in inhibiting the conversion of IP3 to IP4. Certain compounds of Formula I provided herein have an IC₅₀ of less than 20 nM in inhibiting the conversion of IP3 to IP4. Certain compounds of Formula I provided herein have an IC₅₀ of less than 10 nM in inhibiting the conversion of IP3 to IP4. Certain compounds of Formula I provided herein have an IC₅₀ of less than 5 nM in inhibiting the conversion of IP3 to IP4. Certain compounds of Formula I provided herein have an IC₅₀ greater than 10 μM in inhibiting the conversion of IP3 to IP4.

Compounds of Formula I preferably have an IC₅₀ of less than 500 nM, preferably less than 250 nM, more preferably less than 100 nM at inhibiting the phosphorylation of IP3.

By way of example only, the compound (R)-5-(4-((3-methyl-4-(5-(trifluoromethyl)pyridin-2-yl)piperazin-1-yl)methyl)-1H-pyrazol-3-yl)-1H-pyrrole-3-carbonitrile (Example 1) has an IC₅₀ of 9 nM in inhibiting the phosphorylation of IP3 to IP4.

By way of example only, the compound (R)-5-(4-((3-methyl-4-(5-(trifluoromethyl)pyridin-2-yl)piperazin-1-yl)methyl)-1H-pyrazol-3-yl)-1H-pyrrole-3-carboxamide (Example 2) has an IC₅₀ of 3 nM in inhibiting the phosphorylation of IP3 to IP4.

By way of example only, the compound (R)—N-methyl-5-(4-((3-methyl-4-(5-(trifluoromethyl)pyridin-2-yl)piperazin-1-yl)methyl)-1H-pyrazol-3-yl)-1H-pyrrole-3-carboxamide (Example 3) has an IC₅₀ of 11 nM in inhibiting the phosphorylation of IP3 to IP4.

By way of example only, the compound (R)-5-(4-((3-methyl-4-(5-(trifluoromethyl)pyridin-2-yl)piperazin-1-yl)methyl)-1H-pyrazol-3-yl)-1H-pyrrole-2-carbonitrile (Example 4) has an IC₅₀ of 81 nM in inhibiting the phosphorylation of IP3 to IP4.

By way of example only, the compound (R)-5-(4-((3-methyl-4-(5-(trifluoromethyl)pyridin-2-yl)piperazin-1-yl)methyl)-1H-pyrazol-3-yl)-1H-pyrrole-2-carboxamide (Example 5) has an IC₅₀ of 28 nM in inhibiting the phosphorylation of IP3 to IP4.

By way of example only, the compound (R)-4-(4-((3-methyl-4-(5-(trifluoromethyl)pyridin-2-yl)piperazin-1-yl)methyl)-1H-pyrazol-3-yl)-1H-pyrrole-2-carbonitrile (Example 6) has an IC₅₀ of 6 nM in inhibiting the phosphorylation of IP3 to IP4.

By way of example only, the compound (R)-4-(4-((3-methyl-4-(5-(trifluoromethyl)pyridin-2-yl)piperazin-1-yl)methyl)-1H-pyrazol-3-yl)-1H-pyrrole-2-carboxamide (Example 7) has an IC₅₀ of 2 nM in inhibiting the phosphorylation of IP3 to IP4.

By way of example only, the compound (R)-4-(4-((3-methyl-4-(5-(trifluoromethyl)pyridin-2-yl)piperazin-1-yl)methyl)-1H-pyrazol-3-yl)-1H-pyrrole-2-carboxamide (Example 7) has an IC₅₀ of 2 nM in inhibiting the phosphorylation of IP3 to IP4.

By way of example only, the IC₅₀ for inhibiting the phosphorylation of IP3 to IP4 by certain other compounds of Formula (I) are listed in Table 1 and in Table 2 below. In Table 2 the identifying number for each compound is the Example number in the synthetic schemes provided herein.

TABLE 2

| Example | IC50 (nM) |
|---|---|
| 8 | 192 |
| 9 | 278 |
| 10 | 10 |
| 11 | 1480 |
| 12 | 475 |
| 13 | 102 |
| 14 | 24 |
| 15 | 1233 |
| 16 | 975 |
| 17 | 531 |
| 18 | 449 |
| 19 | 40 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-57. (canceled)

58. A compound of Formula (I), or pharmaceutically acceptable salt or pharmaceutically acceptable solvate thereof: 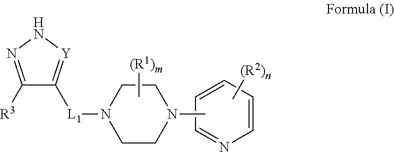
wherein:
L₁ is —(CR¹¹R¹²)ₚ—, —C(O)—, or —S(O)₂—;
L₂ is —C(O)—, —C(O)NR⁵— or —NR⁵C(O;
Y is N or CR⁴;
each R¹ is independently selected from —C(O)R⁹, C₁-C₆alkyl, C₁-C₆heteroalkyl, C₁-C₆haloalkyl, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, aryl, heteroaryl, C₃-C₈cycloalkyl, and C₃-C₁₀heterocycloalkyl groups of R¹ are each optionally substituted with 1 to 3 substituents independently selected from halogen, —CN, C₁-C₆alkyl, C₁-C₆haloalkyl, C₁-C₆alkoxy, C₁-C₆haloalkoxyl, C₃-C₈cycloalkyl, C₃-C₁₀heterocycloalkyl, —OR⁹, —C(O)R⁹, —OC(O)R⁹, —C(O)OR⁹, —N(R⁶R⁷⁾, —C(O)N(R⁶R⁷), —S(O)₂R⁹, —S(O)₂N(R⁶R⁷), and —NR⁷S(O)₂R⁹;
or two R₁ groups are each independently C₁-C₄alkyl and form a C₁-C₄alkyl bridge, or two R₁ groups are each independently C₁-C₄alkyl and taken together with the C atom to which they are attached form an optionally substituted C₃-C₈cycloalkyl;
each R² is independently selected from halogen, —CN, —OR⁹, —C(O)R⁹, —C(O)N(R⁶R⁷), C₁-C₆alkyl, C₁-C₆heteroalkyl, C₁-C₆haloalkyl, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, aryl, heteroaryl, C₃-C₈cycloalkyl, and C₃-C₁₀heterocycloalkyl groups of R² are each optionally substituted with 1 to 3 substituents independently selected from halogen, —CN, C₁-C₆alkyl, C₁-C₆haloalkyl, C₁-C₆ alkoxy, C₁-C₆haloalkoxyl, C₃-C₈cycloalkyl, C₃-C₁₀heterocycloalkyl, —OR⁹, —C(O)R⁹, —OC(O)9-C(O)OR⁹, —N(R⁶R⁷), —C(O)N(R⁶R⁷), —S(O)₂R⁹, —S(O)₂N(R⁶R⁷), and —NR⁷S(O)₂R⁹;
when Y is N then R³ is selected from L₂-R¹⁰, C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, C₃-C₈cycloalkyl, C₃-C₁₀heterocycloalkyl, C₆₋₁₀aryl and C₂-C₉heteroaryl, wherein the C₁-C₆alkyl, C₁-C₆heteroalkyl, C₁-C₆haloalkyl, C₁-C₆alkoxy, C₁-C₆haloalkoxy, C₂-C₉heteroaryl, C₃-C₈cycloalkyl, aryl and C₃-C₁₀heterocycloalkyl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —NR⁶C(O)R⁷, —C(O)N(R⁶R⁷), —S(O)₂R⁹, —S(O)₂N(R⁶R⁷) and —NR⁷S(O)₂R⁹;
when Y is CR⁴ then R³ is selected from L₂-R¹⁰, C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, C₃-C₈cycloalkyl, C₃-C₁₀heterocycloalkyl and C₂-C₉heteroaryl, provided that R³ is not a six-membered heteroaryl containing 1 to 3 N atoms, and wherein the C₁-C₆alkyl, C₁-C₆heteroalkyl, C₁-C₆haloalkyl, C₁-C₆alkoxy, C₁-C₆haloalkoxy, C₂-C₉heteroaryl, C₃-C₈cycloalkyl and C₃-C₁₀heterocycloalkyl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —NR⁶C(O)R⁷, —C(O)N(R⁶R⁷), —S(O)₂R⁹, —S(O)₂N(R⁶R⁷) and —NR⁷S(O)₂R⁹;
R⁴ is selected from H, —C(O)OR⁹, —C(O)R⁹, —C(O)N(R⁶R⁷), —N(R⁶R⁷), —NR⁶C(O)R⁷, —(CH₂)ₙOR⁷, C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₁₀heterocycloalkyl groups of R⁵ are each optionally substituted with 1 to 3 substituents independently selected from halogen, —CN, —R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷), and —NR⁷S(O)₂R⁹;
R⁵, R⁶ and R⁷ are each independently selected from H, C₁-C₆alkyl, C₁-C₆haloalkyl, C₁-C₆alkoxy, C₃-C₈cycloalkyl, C₃-C₁₀heterocycloalkyl, C₁-C₆haloalkyl, C₁-C₆haloalkoxy, aryl and heteroaryl, wherein the C₁-C₆alkyl, C₁-C₆haloalkyl, C₁-C₆alkoxy, C₃-C₈cycloalkyl, C₃-C₁₀heterocycloalkyl, C₁-C₆haloalkyl, C₁-C₆haloalkoxy, aryl and heteroaryl of R⁵, R⁶ and R⁷ are each optionally substituted with 1 to 3 substituents independently selected from halogen, —CN, —R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷), and —NR⁷S(O)₂R⁹,
or R⁶ and R⁷ are each independently C₁-C₄alkyl and taken together with the C atom to which they are attached form a C₃-C₈cycloalkyl;
R⁸ is selected from H, CN, —OR⁹, —C(O)R⁹, —C(O)OR⁹, —C(O)N(R⁶R⁷), —C(═NH)N(R⁶R⁷), C₁-C₆alkyl, C₁-C₆haloalkyl, C₁-C₆alkoxy, C₁-C₆haloalkoxy, C₃-C₈cycloalkyl, and C₃-C₁₀heterocycloalkyl;
R⁹ is selected from H, C₁-C₆alkyl, C₁-C₆haloalkyl, C₁-C₆alkoxy, C₃-C₈cycloalkyl, C₃-C₁₀heterocycloalkyl, C₁-C₆haloalkyl and C₁-C₆haloalkoxy;
R¹⁰ is selected from C₁-C₆alkyl, C₂-C₈alkene, C₂-C₈alkyne, C₁-C₆heteroalkyl, C₁-C₆haloalkyl, C₁-C₆alkoxy, C₁-C₆haloalkoxy, aryl, heteroaryl, C₃-C₈cycloalkyl, and C₃-C₁₀heterocycloalkyl, wherein the C₁-C₆alkyl, C₁-C₆heteroalkyl, C₁-C₆haloalkyl, C₁-C₆alkoxy, C₁-C₆haloalkoxy, aryl, heteroaryl, C₃-C₈cycloalkyl, and C₃-C₁₀heterocycloalkyl groups of R¹⁰ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁷, —S(O)₂N(R⁶R⁷) and —NR⁷S(O)₂R⁹;
R¹¹ and R¹² are each independently selected from H, C₁-C₄alkyl, C₁-C₄heteroalkyl, C₁-C₄haloalkyl, C₁-C₄alkoxy and C₁-C₄haloalkoxy;
or R¹¹ and R¹² are each independently C₁-C₄alkyl and taken together with the C atom to which they are attached form a C₃-C₈cycloalkyl;
m is, independently at each occurrence, 0, 1, 2, 3 or 4;
n is, independently at each occurrence, 0, 1, 2, 3 or 4, and
p is, independently at each occurrence, 1, 2, 3 or 4.

59. The compound of claim 58, wherein the compound of Formula (I) has a structure of Formula (II):

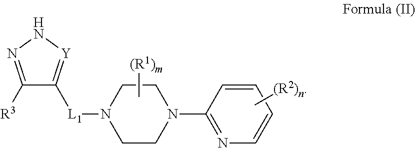

60. The compound of claim 59, wherein n is 0, 1 or 2 and m is 0, 1 or 2.

61. The compound of claim 60, wherein the compound has a structure of Formula (III), Formula (IV) or Formula (V):

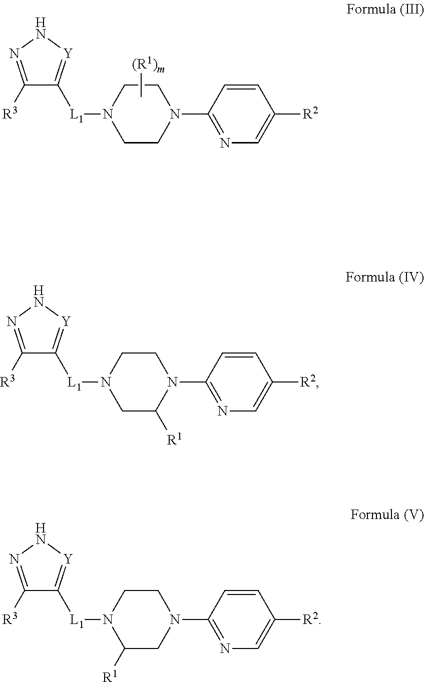

62. The compound of claim 61, wherein L₁ is —(CR¹¹R¹²)ₚ—, p is 1 or 2, and R¹¹ and R¹² are each independently selected from H and C₁-C₄alkyl.

63. The compound of claim 62, wherein L₁ is —(CH₂)—.

64. The compound of claim 63, wherein the compound has a structure of Formula (VI), Formula (VII), Formula (VIII), Formula (IX), Formula (X) or Formula (XI):

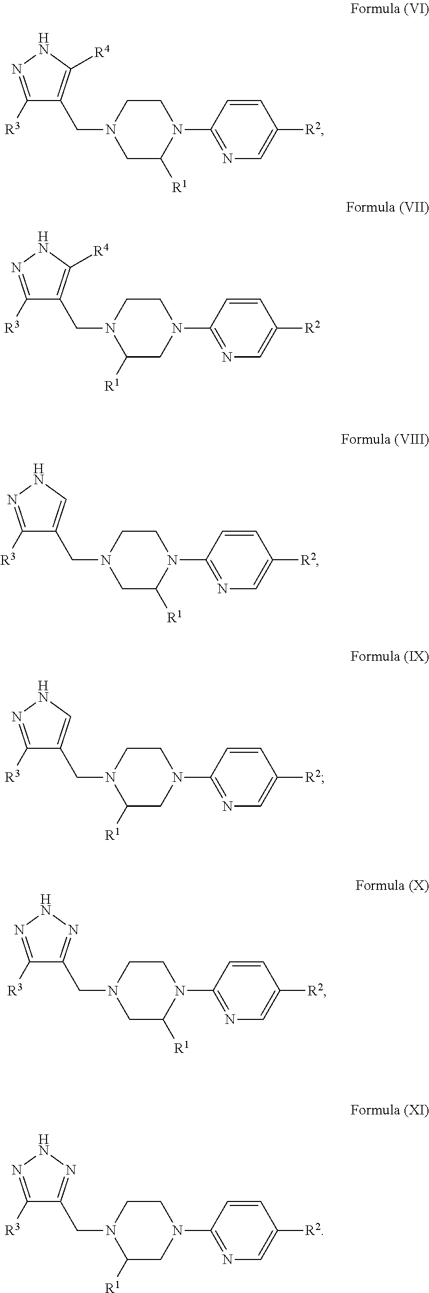

65. The compound of claim 64, wherein R¹ is C₁-C₆alkyl or C₁-C₆haloalkyl.

66. The compound of claim 65, wherein R² is C₁-C₆alkyl or C₁-C₆haloalkyl.

67. The compound of claim 66, wherein R¹ is methyl, ethyl, trifluoromethyl, difluoromethyl or fluoromethyl.

68. The compound of claim 67, wherein R² is methyl, ethyl, trifluoromethyl, difluoromethyl or fluoromethyl.

69. The compound of claim 68, wherein the compound has a structure of Formula (XII), Formula (XIII), Formula (XIV), Formula (XV), Formula (XVI), Formula (XVII), Formula (XVIII) or Formula (XIX):

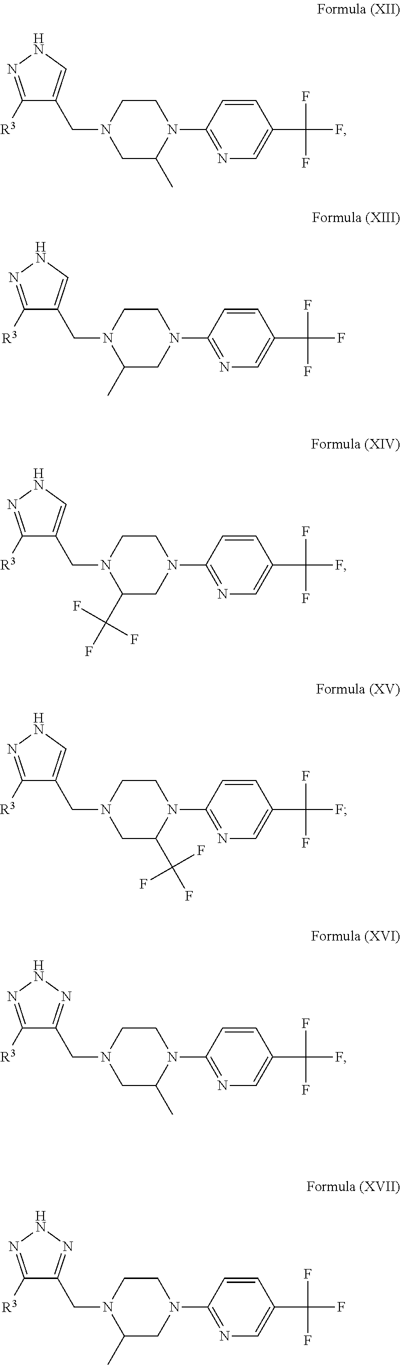

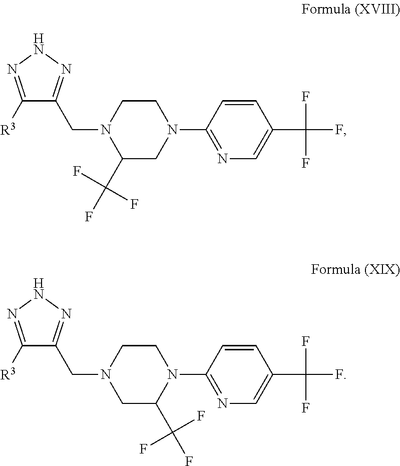

70. The compound of claim 69, wherein R³ is C₃-C₁₀heterocycloalkyl or C₂-C₉heteroaryl, wherein the C₃-C₁₀heterocycloalkyl and C₂-C₉heteroaryl groups of R³ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁹, —S(O)₂N(R⁶R⁷) and —NR⁷S(O)₂R⁹ and provided that R³ is not a six-membered heteroaryl containing 1 to 3 N atoms.

71. The compound of claim 70, wherein R³ is selected from oxo-1,2-dihydropyridine, 4,5,6,7-tetrahydro-1H-benzo[d]imidazolyl, 5,6-dihydropyridin-1(2H)-yl, 1,2,3,6-tetrahydropyridin-4-yl, piperidinyl, benzimidazolyl, furyl, imidazolyl, imidazo[4,5-c]pyridinyl, indolyl, isoquinolinyl, pyrazolyl, pyrrolyl, pyrrolo[2,3-b]pyridinyl and thienyl.

72. The compound of claim 71, wherein R³ is substituted with 1 to 3 substituents independently selected from halogen and R⁸ , wherein R⁸ is selected from C₁-C₆alkyl, C₁-C₆haloalkyl, H, —CN, —OR⁹, —C(O)R⁹, —C(O)OR⁹, —C(O)N(R⁶R⁷), and —C(═NH)N(R⁶R⁷).

73. The compound of claim 72, wherein R³ is selected from isoquinoline, 2-oxo-1,2-dihydropyridine-4-carbonitrile, thiophene, pyrrole, 1H-pyrrole-3-carbonitrile, benzimidazole, 5-fluoro-1H-benzo[d]imidazole, 4,5,6,7-tetrahydro-1H-benzo[d]imidazole, imidazole, 5-methyl-1H-imidazole, 4,5-dimethyl-1H-imidazole, 4-cyano-1H-pyrazole, 1H-imidazo[4,5-c]pyridine, 4-(trifluoromethyl)-1H-imidazole, 1H-benzo[d]imidazole-5-carbonitrile, 1H-imidazole-4-carbonitrile, 1H-pyrrole-3-carboxamide, 1H-pyrrole-2-carboxamide, 1H-pyrrole-2-carbonitrile, furan-2-carboxylic acid, furan-2-carboxamide, furan-3-carboxylic acid, furan-3-carboxamide, furan-2-carboxylate, methyl furan-2-carboxylate, N-methyl-1H-pyrrole-3-carboxamide, 1H-pyrrolo[2,3-b]pyridine, N,N-dimethyl-1H-pyrrole-3-carboxamide, N-(2-hydroxypropyl)-1H-pyrrole-3-carboxamide, (S)—N-(1-hydroxypropan-2-yl)-1H-pyrrole-3-carboxamide, 1H-indole, N-(2-hydroxyethyl)-1H-pyrrole-3-carboxamide, 1,2,3,6-tetrahydropyridine, 5,6-dihydropyridine-1(2H)-carbaldehyde, 1-(5,6-dihydropyridin-1(2H)-yl)ethanone, piperidine, 1-(piperidin-1-yl)ethanone, piperidine-1-carbaldehyde, 1H-imidazole-4-carboximidamide and 1H-imidazole-4-carboxamide.

74. The compound of claim 73, wherein R³ is L₂-R¹⁰.

75. The compound of claim 74, wherein L₂ is selected from C₁-C₆alkenylene, —C(O)— and —C(O)NR⁵—.

76. The compound of claim 75, wherein R¹⁰ is selected from aryl, heteroaryl and C₃-C₁₀heterocycloalkyl, wherein the aryl, heteroaryl and C₃-C₁₀heterocycloalkyl groups of R¹⁰ are each optionally substituted with 1 to 3 substituents independently selected from halogen, —CN, R⁸, —OR⁹, —C(O)R⁹, —OC(O)R⁹, —C(O)OR⁹, —N(R⁶R⁷), —C(O)N(R⁶R⁷), —S(O)₂R⁷, —S(O)₂N(R⁶R⁷) and —NR⁷S(O)₂R⁹.

77. The compound of claim 76, wherein L₂ is —C(O)NR⁵— and R¹⁰ is selected from 1H-indole, pyridine, 1H-imidazole-5-carbonitrile and 1H-pyrazole-4-carbonitrile.

78. The compound of claim 77, wherein L₂ is —C(O)— and R¹⁰ is selected from azetidin-3-ol, pyrrolidin-3-ol and piperidin-4-ol.

79. The compound of claim 58 selected from:
(R)-4-((3-methyl-4-(5-(trifluoromethyl)pyridin-2-yl)piperazin-1-yl)methyl)-N-(pyridin-4-yl)-1H-pyrazole-3-carboxamide; (R)-5-(4-((3-methyl-4-(5-(trifluoromethyl)pyridin-2-yl)piperazin-1-yl)methyl)-1H-pyrazol-3-yl)-1H-pyrrole-3-carbonitrile; (R)-5-(4-((3-methyl-4-(5-(trifluoromethyl)pyridin-2-yl)piperazin-1-yl)methyl)-1H-pyrazol-3-yl)-1H-pyrrole-3-carboxamide; (R)—N-methyl-5-(4-((3-methyl-4-(5-(trifluoromethyl)pyridin-2-yl)piperazin-1-yl)methyl)-1H-pyrazol-3-yl)-1H-pyrrole-3-carboxamide; (R)-5-(4-((3-methyl-4-(5-(trifluoromethyl)pyridin-2-yl)piperazin-1-yl)methyl)-1H-pyrazol-3-yl)-1H-pyrrole-2-carbonitrile; (R)-5-(4-((3-methyl-4-(5-(trifluoromethyl)pyridin-2-yl)piperazin-1-yl)methyl)-1H-pyrazol-3-yl)-1H-pyrrole-2-carboxamide; (R)-4-(4-((3-methyl-4-(5-(trifluoromethyl)pyridin-2-yl)piperazin-1-yl)methyl)-1H-pyrazol-3-yl)-1H-pyrrole-2-carbonitrile; (R)-4-(4-((3-methyl-4-(5-(trifluoromethyl)pyridin-2-yl)piperazin-1-yl)methyl)-1H-pyrazol-3-yl)-1H-pyrrole-2-carboxamide; (R)-2-(4-((3-methyl-4-(5-(trifluoromethyl)pyridin-2-yl)piperazin-1-yl)methyl)-1H-pyrazol-3-yl)-1H-indole; (R)-2-methyl-4-((3-(1,2,3,6-tetrahydropyridin-4-yl)-1H-pyrazol-4-yl)methyl)-1-(5-(trifluoromethyl)pyridin-2-yl)piperazine; (R)-4-(4-((3-methyl-4-(5-(trifluoromethyl)pyridin-2-yl)piperazin-1-yl)methyl)-1H-pyrazol-3-yl)-5,6-dihydropyridine-1(2H)-carbaldehyde; (R)-2-methyl-4-((3-(4-(trifluoromethyl)-1H-imidazol-2-yl)-1H-pyrazol-4-yl)methyl)-1-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-1H-imidazole-4-carboximidamide; (R)-2-(4-((3-methyl-4-(5-(trifluoromethyl)pyridin-2-yl)piperazin-1-yl)methyl)-1H-pyrazol-3-yl)-1H-imidazo[4,5-c]pyridine; (R,Z)-5-((4-((3-methyl-4-(5-(trifluoromethyl)pyridin-2-yl)piperazin-1-yl)methyl)-1H-pyrazol-3-yl)methylene)imidazolidine-2,4-dione; (R)-6-(4-((3-methyl-4-(5-(trifluoromethyl)pyridin-2-yl)piperazin-1-yl)methyl)-1H-pyrazol-3-yl)isoquinoline; (R)-6-(4-((3-methyl-4-(5-(trifluoromethyl)pyridin-2-yl)piperazin-1-yl)methyl)-1H-pyrazol-3-yl)-2-oxo-1,2-dihydropyridine-3-carbonitrile; 1-((3-(thiophen-2-yl)-1H-pyrazol-4-yl)methyl)-4-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-1H-benzo[d]imidazole; (R)-2-methyl-4-((3-(5-methyl-1H-imidazol-2-yl)-1H-pyrazol-4-yl)methyl)-1-(5-(trifluoromethyl)pyridin-2-yl)piperazine; (R)-4-((3-(4,5-dimethyl-1H-imidazol-2-yl)-1H-pyrazol-4-yl)methyl)-2-methyl-1-(5-(trifluoromethyl)pyridin-2-yl)piperazine; (R)-2-(4-((3-methyl-4-(5-(trifluoromethyl)pyridin-2-yl)piperazin-1-yl)methyl)-1H-pyrazol-3-yl)-4,5,6,7-tetrahydro-1H-benzo[d]imidazole; (R)-5-fluoro-2-(4-((3-methyl-4-(5-(trifluoromethyl)pyridin-2-yl)piperazin-1-yl)methyl)-1H-pyrazol-3-yl)-1H-benzo[d]imidazole; (R)-2-(4-((3-methyl-4-(5-(trifluoromethyl)pyridin-2-yl)piperazin-1-yl)methyl)-1H-pyrazol-3-yl)-1H-benzo[d]imidazole-5-carbonitrile; (S)-5-(4-((3-(trifluoromethyl)-4-(5-(trifluoromethyl)pyridin-2-yl)piperazin-1-yl)methyl)-1H-pyrazol-3-yl)-1H-pyrrole-3-carbonitrile; (R)—N-(5-cyano-1H-imidazol-4-yl)-4-((3-methyl-4-(5-(trifluoromethyl)pyridin-2-yl)piperazin-1-yl)methyl)-1H-pyrazole-3-carboxamide; (R)—N-(4-cyano-1H-pyrazol-3-yl)-4-((3-methyl-4-(5-(trifluoromethyl)pyridin-2-yl)piperazin-1-yl)methyl)-1H-pyrazole-3-carboxamide; (R)-(3-hydroxyazetidin-1-yl)(4-((3-methyl-4-(5-(trifluoromethyl)pyridin-2-yl)piperazin-1-yl)methyl)-1H-pyrazol-3-yl)methanone; (3-hydroxypyrrolidin-1-yl)(4-(((R)-3-methyl-4-(5-(trifluoromethyl)pyridin-2-yl)piperazin-1-yl)methyl)-1H-pyrazol-3-yl)methanone; (R)-(4-hydroxypiperidin-1-yl)(4-((3-methyl-4-(5-(trifluoromethyl)pyridin-2-yl)piperazin-1-yl)methyl)-1H-pyrazol-3-yl)methanone; (R)-methyl 5-(4-((3-methyl-4-(5-(trifluoromethyl)pyridin-2-yl)piperazin-1-yl)methyl)-1H-pyrazol-3-yl)furan-2-carboxylate; (R)-5-(4-((3-methyl-4-(5-(trifluoromethyl)pyridin-2-yl)piperazin-1-yl)methyl)-1H-pyrazol-3-yl)furan-2-carboxylic acid; (R)-5-(4-((3-methyl-4-(5-(trifluoromethyl)pyridin-2-yl)piperazin-1-yl)methyl)-1H-pyrazol-3-yl)furan-2-carboxamide; (R)-4-(4-((3-methyl-4-(5-(trifluoromethyl)pyridin-2-yl)piperazin-1-yl)methyl)-1H-pyrazol-3-yl)-1H-pyrrolo[2,3-b]pyridine; (R)—N,N-dimethyl-5-(4-((3-methyl-4-(5-(trifluoromethyl)pyridin-2-yl)piperazin-1-yl)methyl)-1H-pyrazol-3-yl)-1H-pyrrole-3-carboxamide; (R)—N-(2-hydroxyethyl)-5-(4-((3-methyl-4-(5-(trifluoromethyl)pyridin-2-yl)piperazin-1-yl)methyl)-1H-pyrazol-3-yl)-1H-pyrrole-3-carboxamide; N-(2-hydroxypropyl)-5-(4-(((R)-3-methyl-4-(5-(trifluoromethyl)pyridin-2-yl)piperazin-1-yl)methyl)-1H-pyrazol-3-yl)-1H-pyrrole-3-carboxamide; (R)-3-(4-((3-methyl-4-(5-(trifluoromethyl)pyridin-2-yl)piperazin-1-yl)methyl)-1H-pyrazol-3-yl)-1H-pyrrolo[2,3-b]pyridine; N—((S)-1-hydroxypropan-2-yl)-5-(4-(((R)-3-methyl-4-(5-(trifluoromethyl)pyridin-2-yl)piperazin-1-yl)methyl)-1H-pyrazol-3-yl)-1H-pyrrole-3-carboxamide; (R,Z)-5-((4-((3-methyl-4-(5-(trifluoromethyl)pyridin-2-yl)piperazin-1-yl)methyl)-1H-pyrazol-3-yl)methylene)-2-thioxoimidazolidin-4-one; (R)-1-(4-(4-((3-methyl-4-(5-(trifluoromethyl)pyridin-2-yl)piperazin-1-yl)methyl)-1H-pyrazol-3-yl)-5,6-dihydropyridin-1(2H)-yl)ethanone; (R)-2-methyl-4-((3-(piperidin-4-yl)-1H-pyrazol-4-yl)methyl)-1-(5-(trifluoromethyl)pyridin-2-yl)piperazine; (R)-4-(4-((3-methyl-4-(5-(trifluoromethyl)pyridin-2-yl)piperazin-1-yl)methyl)-1H-pyrazol-3-yl)piperidine-1-carbaldehyde; (R,Z)-2-imino-5-((4-((3-methyl-4-(5-(trifluoromethyl)pyridin-2-yl)piperazin-1-yl)methyl)-1H-pyrazol-3-yl)methylene)imidazolidin-4-one; (S)-5-(4-((3-(trifluoromethyl)-4-(5-(trifluoromethyl)pyridin-2-yl)piperazin-1-yl)methyl)-1H-pyrazol-3-yl)-1H-pyrrole-3-carboxamide; (R)-5-(4-((3-methyl-4-(5-(trifluoromethyl)pyridin-2-yl)piperazin-1-yl)methyl)-1H-pyrazol-3-yl)furan-3-carboxylic acid; (R)-5-(4-((3-methyl-4-(5-(trifluoromethyl)pyridin-2-yl)piperazin-1-yl)methyl)-1H-pyrazol-3-yl)furan-3-carboxamide; (R)-2-(4-((3-methyl-4-(5-(trifluoromethyl)pyridin-2-yl)piperazin-1-yl)methyl)-1H-pyrazol-3-yl)-1H-imidazole-4-carbonitrile; (R)-2-(4-((3-methyl-4-(5-(trifluoromethyl)pyridin-2-yl)piperazin-1-yl)methyl)-1H-pyrazol-3-yl)-1H-imidazole-4-carboxamide; (R)-4-(5-((3-methyl-4-(4-(trifluoromethyl)phenyl)piperazin-1-yl)methyl)-2H-1,2,3-triazol-4-yl)benzonitrile. (R)-6-(4-((3-methyl-4-(5-(trifluoromethyl)pyridin-2-yl)piperazin-1-yl)methyl)-1H-pyrazol-3-yl)-2-oxo-1,2-dihydropyridine-4-carbonitrile; (R)-4-((3-methyl-4-(5-(trifluoromethyl)pyridin-2-yl)piperazin-1-yl)methyl)-N-(3-oxo-3,4-dihydro-2H-benzo[b][1,4]oxazin-7-yl)-1H-pyrazole-3-carboxamide; (R)-4-((3-methyl-4-(5-(trifluoromethyl)pyridin-2-yl)piperazin-1-yl)methyl)-N-(3-oxo-3,4-dihydro-2H-benzo[b][1,4]oxazin-6-yl)-1H-pyrazole-3-carboxamide; (R)-4-((3-methyl-4-(5-(trifluoromethyl)pyridin-2-yl)piperazin-1-yl)methyl)-N-(4-(N-(5-methylisoxazol-3-yl)sulfamoyl)phenyl)-1H-pyrazole-3-carboxamide; (R)-3-(4-((3-methyl-4-(5-(trifluoromethyl)pyridin-2-yl)piperazin-1-yl)methyl)-1H-pyrazol-3-yl)-1H-indole, and (R)-4-(5-((3-methyl-4-(5-(trifluoromethyl)pyridin-2-yl)piperazin-1-yl)methyl)-2H-1,2,3-triazol-4-yl)benzonitrile.

80. A pharmaceutical composition comprising a therapeutically effective amount of a compound of Formula (I) of claim 58 and a pharmaceutically acceptable carrier.

81. A method for treating a disease or disorder where modulation of B lymphocyte development and function is implicated, comprising administering to a human in need of such treatment an effective amount of a compound of Formula (I) of claim 58, or pharmaceutically acceptable salts or pharmaceutical compositions thereof.

82. The method of claim 81, wherein the disease or condition is an autoimmune disease.

83. The method of claim 82, wherein the autoimmune disease is rheumatoid arthritis, systemic lupus erythematosus, idiopathic thrombocytopenic purpura, hemolytic anemia, or psoriasis.

84. A method for treating a cell-proliferative condition, comprising administering to a human in need of such treatment an effective amount of a compound of Formula (I) of claim 58, or pharmaceutically acceptable salts or pharmaceutical compositions thereof; wherein the cell-proliferative condition is lymphoma.

85. The method of claim 84, wherein the lymphoma is B cell lymphoma.
